(19) United States
(12) Patent Application Publication
(10) Pub. No.: US 2009/0048112 A1
(43) Pub. Date: Feb. 19, 2009

(54) Novel Herbicides

(75) Inventors: Alison Clare Elliott, Bracknell (GB); Philip Hughes, Bracknell (GB); Andrew Plant, Bracknell (GB)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA LIMITED, Guildford, Surrey (GB)

(21) Appl. No.: 11/913,983

(22) PCT Filed: Apr. 11, 2006
(86) PCT No.: PCT/GB2006/001316

§ 371 Date: May 16, 2008

(30) Foreign Application Priority Data
May 18, 2005 (GB) 0510151.4

(51) Int. Cl. A01N 43/78; C07D 277/20; A01P 13/00

(52) U.S. Cl. 504/266; 548/186

(57) ABSTRACT

Compounds of formula (I) wherein the substituents are defined as in claim 1, are suitable for use as herbicides. Also claimed is the intermediate (II) wherein R¹ is chloro, R² is hydrogen and X^{A} is methylsulfonate, three processes for the preparation of compounds of formula (Ih) wherein m is 1 or 2, and the other substituents are defined as in claim 1, and a process for the preparation of compounds of the formula (IVa) wherein X^{B} is a halogen atom, and the substitutents are defined as in claim 1.

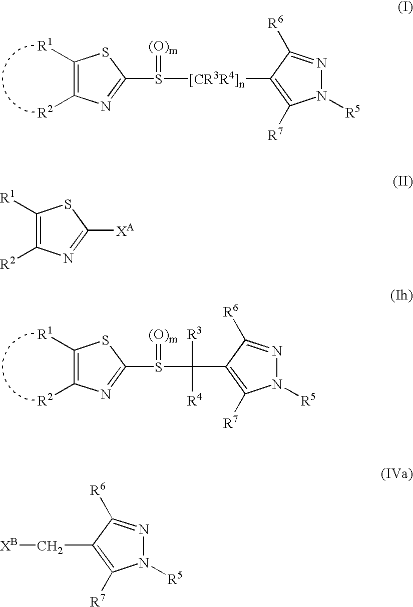

The present invention relates to novel, herbicidally active thiazole compounds, to processes for their preparation, to compositions comprising these compounds, and to their use in controlling weeds, especially in crops of useful plants, or in inhibiting plant growth.

2-(1H-Pyrazol-4-ylmethylsulfanyl)-thiazole compounds have been disclosed as photographic materials in, for example, JP 06-148876, as intermediates in the synthesis of agricultural and horticultural fungicides JP 93-313520 and as intermediates in the synthesis of herbicides JP 86-194795.

Novel 2-(1H-pyrazol-4-ylalkylsulfanyl)-thiazole, 2-(1H-pyrazol-4-ylalkylsulfinyl)-thiazole and 2-(1H-pyrazol-4-ylalkylsulfonyl)-thiazole compounds having herbicidal and growth-inhibiting properties have now been found.

The present invention accordingly relates to compounds of formula I:

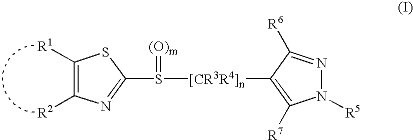

wherein
R¹ and R² are each independently of the other hydrogen, C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCHO, —NHCO—C₁-C₆alkyl, —NHCO—C₁-C₆haloalkyl, —NHCO₂—C₁-C₆alkyl, —NHCO₂—C₁-C₆haloalkyl, —NHCONH—C₁-C₆alkyl, —NHCONH—C₁-C₆haloalkyl, —NHSO₂—C₁-C₆alkyl, —NHSO₂—C₁-C₆haloalkyl, —NHSO₂-phenyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or CONR^{a}R^{b} wherein R^{a} and R^{b} are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R^{a} and R^{b} together form a C₃-C₈alkylene group which optionally contains one oxygen or sulfur atom or one or two amino or C₁-C₆alkylamino groups, or
R¹ and R² together with the carbon atom to which they are bonded form a C₃-C₈alkylene group, which optionally contains one or two oxygen or sulfur atoms or one to three amino or C₁-C₆alkylamino groups, and which optionally contains a double bond and optionally is substituted by one to three substituents independently selected from C₃-C₆cycloalkyl, C₁-C₆haloalkyl, C₁-C₆hydroxyalkyl, pyrrolyl-CH₂—, pyrazolyl-CH₂—, triazolyl-CH₂—, imidazolyl-CH₂—, tetrazolyl-CH₂—, indolyl-CH₂—, indazolyl-CH₂—, benzotriazolyl-CH₂—, C₂-C₆alkenyl, C₂-C₆alkynyl, C₂-C₆haloalkenyl, C₂-C₆alkenyloxy, C₂-C₆alkynyloxy, C₁-C₆alkylcarbonyl, C₁-C₆haloalkylcarbonyl, phenylcarbonyl or phenylcarbonyl substituted by one to three R¹¹, phenoxycarbonyl or phenoxycarbonyl substituted by one to three R¹¹, benzyloxycarbonyl or benzyloxycarbonyl substituted by one to three R¹¹, nitro, formyl, carboxyl, halogen, azido, thiocyanato, tri(C₁-C₆alkyl)silyl, C₁-C₆alkylcarbonyl-C₁-C₂alkyl, C₁-C₆alkoxycarbonyl-C₁-C₂alkyl, cyano-C₁-C₂alkyl, C₁-C₆alkylaminocarbonyl-C₁-C₂alkyl, di-C₁-C₆alkylaminocarbonyl-C₁-C₂alkyl, C₁-C₆alkoxy-C₁-C₂alkyl, C₁-C₂alkyl-P(O)(OC₁-C₆alkyl)₂, C₁-C₂alkyl-NO₂, mercapto, phenylthio or phenylthio substituted by one to three R¹¹, pyridylthio, C₁-C₆alkylthio, C₁-C₆haloalkylthio, C₁-C₆alkylthio-C₁-C₆alkyl, C₁-C₆alkylsulfinyl, C₁-C₆haloalkylsulfinyl, C₁-C₆alkylsulfinyl-C₁-C₆alkyl, C₁-C₆alkylsulfonyl, C₁-C₆haloalkylsulfonyl, C₁-C₆alkylsulfonyl-C₁-C₆alkyl, benzylsulfonyl or benzylsulfonyl substituted by one to three R¹¹, phenylsulfinyl or phenylsulfinyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyl or benzyl substituted by one to three R¹¹, benzyloxy or benzyloxy substituted by one to three R¹¹, —CONH—SO₂—C₁-C₆alkyl, —CONH—SO₂—C₁-C₆haloalkyl, —NHCHO, —NHCO—C₁-C₆alkyl, —NHCO—C₁-C₆haloalkyl, —NHCOO—C₁-C₆alkyl, —NHCOO—C₁-C₆haloalkyl, —NHCONH—C₁-C₆alkyl, —NHCONH—C₁-C₆haloalkyl, —NHSO₂—C₁-C₆alkyl, —NHSO₂—C₁-C₆haloalkyl, —NHSO₂-phenyl, —OCO—C₁-C₆alkyl, —OCO—C₁-C₆haloalkyl, —OCO-phenyl or —OCO-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₃-C₆cycloalkyl, C₁-C₆haloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkylamino groups, or
phenyl or naphthyl, which is optionally substituted by one to three substituents independently selected from C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkylamino groups, or
a 5- to 10-membered heteroaryl containing one to three nitrogen, oxygen or sulfur atoms, which is optionally benzo-fused, and which is optionally substituted by one to three substituents independently selected from C₁-C₆alkyl, C₃-C₆cycloalkyl, C₁-C₆haloalkyl, C₁-C₆-hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkylamino groups, or
R¹ and R² join together with the carbon atoms to which they are bonded to form a fused aromatic ring, which is optionally substituted by one to three substituents independently selected from C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkyl amino groups, or
R¹ and R² join together with the carbon atoms to which they are bonded to form a fused heterocyclic ring containing one to three nitrogen, oxygen or sulfur atoms which is optionally substituted by one to three substituents independently selected from C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkylamino groups;
R³ and R⁴ are each independently of the other hydrogen, C₁-C₆alkyl, C₁-C₆haloalkyl, halogen, cyano, C₁-C₆alkoxycarbonyl;
m is 0, 1 or 2;
n is 1, 2 or 3;
R⁵, R⁶ and R⁷ are each independently of the others hydrogen, hydroxyl, mercapto, halogen, C₁-C₁₀alkyl or C₁-C₁₀alkyl substituted by one R⁸, C₁-C₄haloalkyl, C₃-C₈cycloalkyl, C₁-C₁₀alkoxy or C₁-C₁₀alkoxy substituted by one R⁹, C₁-C₄haloalkoxy, C₃-C₈cycloalkyloxy, C₃-C₈cycloalkylC₁-C₃alkoxy, C₁-C₁₀alkylthio or C₁-C₁₀alkylthio substituted by one R⁹, C₁-C₄haloalkylthio, C₂-C₆alkenyl, C₂-C₆haloalkenyl, C₂-C₆alkenyloxy, C₂-C₆alkynyl, C₂-C₆alkynyloxy, C₁-C₁₀alkylsulfinyl or C₁-C₁₀alkylsulfinyl substituted by R⁹, C₁-C₁₀alkylsulfonyl or C₁-C₁₀alkylsulfonyl substituted by one R⁹, C₁-C₄haloalkylsulfinyl, C₁-C₁₀alkylsulfonyloxy substituted by one R⁹, C₁-C₄haloalkylsulfonyl, C₁-C₁₀alkylsulfonyloxy, C₁-C₄haloalkylsulfonyloxy, phenyl or phenyl substituted by one to three R¹⁰, phenoxy or phenoxy substituted by one to three R¹⁰, phenylthio or phenylthio substituted by one to three R¹⁰, heteroaryl or heteroaryl substituted by one to three R¹⁰, heteroaryloxy or heteroaryloxy substituted by one to three R¹⁰, heteroarylthio or heteroarylthio substituted by one to three R¹⁰, phenylsulfinyl or phenylsulfinyl substituted by one to three R¹⁰, phenylsulfonyl or phenylsulfonyl substituted by one to three R¹⁰, heteroarylsulfinyl or heteroarylsulfinyl substituted by one to three R¹⁰, heteroarylsulfonyl or heteroarylsulfonyl substituted by one to three R¹⁰, phenylsulfonyloxy or phenylsulfonyloxy substituted by one to three R¹⁰, C₁-C₆alkylcarbonyl, C₁-C₄haloalkylcarbonyl, C₃-C₈cycloalkylcarbonyl, benzylcarbonyl or benzylcarbonyl substituted by one to three R¹⁰, phenylcarbonyl or phenylcarbonyl substituted by one to three R¹⁰, carboxyl, C₁-C₁₀alkoxycarbonyl, benzyloxycarbonyl or benzyloxycarbonyl substituted by one to three R¹⁰, phenoxycarbonyl or phenoxycarbonyl substituted by one to three R¹⁰, cyano, —CONR^{c}R^{d} (wherein R^{c} and R^{d} are each independently of the other hydrogen, C₁-C₁₀alkyl, phenyl or phenyl substituted by one to three R¹⁰), —O(CO)C₁-C₆alkyl, —O(CO)C₁-C₄haloalkyl, —O(CO)benzyl or —O(CO)benzyl substituted by one to three R¹⁰, —O(CO)phenyl or —O(CO)phenyl substituted by one to three R¹⁰, nitro, or —NR^{c}R^{d} (wherein R^{c} and R^{d} are each independently of the other hydrogen, C₁-C₁₀alkyl, phenyl or phenyl substituted by one to three R¹⁰, C₁-C₆alkylcarbonyl, C₁-C₄haloalkylcarbonyl, benzylcarbonyl or benzylcarbonyl substituted by one to three R¹⁰, phenylcarbonyl or phenylcarbonyl substituted by one to three R¹⁰, C₁-C₁₀alkylsulfonyl, C₁-C₄haloalkylsulfonyl, benzylsulfonyl or benzylsulfonyl substituted by one to three R¹⁰, and phenylsulfonyl or phenylsulfonyl substituted by one to three R¹⁰), or
when R⁵ and R⁷ are substituted both by alkyl, both by alkoxy, alkyl and alkoxy, alkyl and alkylthio, alkyl and alkylsulfonyl, alkyl and monoalkylamino, alkyl and dialkylamino, the two groups optionally form together with the atoms to which they bond, a 5- to 8-membered ring which is optionally substituted by 1 to 4 halogen atoms;
R⁸ is hydroxy, C₃-C₈cycloalkyl or C₃-C₈cycloalkyl substituted by halogen or by C₁-C₁₀alkyl, C₁-C₁₀alkoxy, C₁-C₁₀alkylthio, C₁-C₁₀alkylsulfonyl, C₁-C₁₀alkoxycarbonyl, C₂-C₆haloalkenyl, —NR^{e}R^{f} (wherein R^{e} and R^{f} are each independently of the other hydrogen, C₁-C₁₀alkyl, C₁-C₆alkylcarbonyl, C₁-C₄haloalkylcarbonyl, C₁-C₁₀alkylsulfonyl, C₁-C₄haloalkylsulfonyl), —CONR^{e}R^{f} (wherein R^{e} and R^{f} are each independently of the other hydrogen, C₁-C₁₀alkyl, phenyl or phenyl substituted by one to three R¹⁰), C₁-C₆alkylcarbonyl, C₁-C₄haloalkylcarbonyl, cyano, phenyl or phenyl substituted by one to three R¹⁰, or phenoxy or phenoxy substituted by one to three R¹⁰;
R⁹ is C₁-C₁₀alkoxy, C₁-C₁₀alkoxycarbonyl, phenyl or phenyl substituted by one to three R¹⁰, heteroaryl or heteroaryl substituted by one to three R¹⁰, C₁-C₁₀alkylcarbonyl, C₁-C₁₀haloalkylcarbonyl, cyano, or —CONR^{g}R^{h} (wherein R^{g} and R^{h} are each independently of the other hydrogen, C₁-C₁₀alkyl, phenyl or phenyl substituted by one to three R¹⁰);
R¹⁰ are each independently of the others C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or CONRⁱR^{k} wherein Rⁱ and R^{k} are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or Rⁱ and R^{k} together form a C₃-C₈alkylene group which optionally contains one oxygen or sulfur atom or one or two amino or C₁-C₆alkylamino groups;
R¹¹ are each independently of the others C₁-C₆haloalkyl, C₁-C₆alkoxycarbonyl, nitro, cyano, formyl, carboxyl or halogen;
and to N-oxides, salts and optical isomers of compounds of formula I, with the proviso that where R¹ and R² are fused to form an unsubstituted benzothiazole ring, R³ and R⁴ are hydrogen, n is 1, R⁵ is 3,5-dichlorobenzylcarbonyl, and R⁶ and R⁷ are methyl, then m cannot be 0.

The compounds of the invention may contain one or more asymmetric carbon atoms, for example, in the —CR³R⁴— group and may exist as enantiomers (or as pairs of diastereoisomers) or as mixtures of such. Further, when m is 1, the compounds of the invention are sulfoxides, which can exists in two enantiomeric forms, and the adjacent carbon can also exists in two enantiomeric forms. Compounds of general formula I can therefore exist as racemates, diastereoisomers, or single enantiomers, and the invention includes all possible isomers or isomer mixtures in all proportions. It is to be expected that for any given compound, one isomer may be more herbicidally active than another.

Except where otherwise stated, alkyl groups and alkyl moieties of alkoxy, alkylthio, etc., suitably contain from 1 to 10, typically from 1 to 6, carbon atoms in the form of straight or branched chains. Examples are methyl, ethyl, n- and iso-propyl and n-, sec-, iso- and tert-butyl.

Except where otherwise stated, cycloalkyl groups and cycloalkyl moieties of cycloalkoxy, cycloalkyl-alkoxy, etc., suitably contain from 3 to 8, typically from 3 to 6, carbon atoms. Examples are cyclopropyl, cyclobutyl, cyclopentyl and cyclohexyl. The cycloalkyl radicals may be in bi- or tri-cyclic form.

Except where otherwise stated, haloalkyl groups and haloalkyl moieties of haloalkoxy, haloalkylthio, etc., also suitably contain from 1 to 6, typically from 1 to 4, carbon atoms in the form of straight or branched chains. Examples are difluoromethyl and 2,2,2-trifluoroethyl.

Except where otherwise stated, hydroxyalkyl groups also suitably contain from 1 to 6, typically from 1 to 4, carbon atoms in the form of straight or branched chains. Examples are 1,2-dihydroxyethyl and 3-hydroxypropyl.

Except where otherwise stated, alkenyl and alkynyl moieties also suitably contain from 2 to 6, typically from 2 to 4, carbon atoms in the form of straight or branched chains. Examples are allyl, ethynyl and propargyl.

Except where otherwise stated, haloalkenyl groups and haloalkynyl groups also suitably contain from 2 to 6, typically from 2 to 4, carbon atoms in the form of straight or branched chains. Examples are trifluoroallyl and 1-chloroprop-1-yn-3-yl.

Halo includes fluoro, chloro, bromo and iodo. Most commonly it is fluoro, chloro or bromo and usually fluoro or chloro.

Except where otherwise stated, alkylene groups suitably contain from 1 to 10, typically from 1 to 6, carbon atoms in the form of straight or branched chains. Examples are methylene, ethylene, n- and iso-propylene and n-, sec-, iso- and tert-butylene.

Except where otherwise stated, heteroaryl groups suitably are 5- to 10-membered aromatic rings containing one to three nitrogen, oxygen or sulfur atoms, which may be optionally benzo-fused. Examples are thienyl, furyl, pyrrolyl, isoxazolyl, oxazolyl, thiazolyl, oxadiazolyl, pyrazolyl, imidazolyl, triazolyl, isothiazolyl, thiadiazolyl, pyridyl, pyrimidinyl, pyrazinyl, pyridazinyl, triazinyl, benzofuranyl, benzothienyl, benzothiazolyl, benzoxazolyl, benzimidazolyl, indolyl, quinolyl, isoquinolyl, quinazolinyl and quinoxalinyl groups and, where appropriate, N-oxides and salts thereof.

Except where otherwise stated, heterocyclyl groups suitably are 5- to 10-membered rings containing one to three nitrogen, oxygen or sulfur atoms, which may be optionally benzo-fused. Examples are 1,3-benzodioxolyl and 1, 3-4H-benzodioxinyl groups and, where appropriate, N-oxides and salts thereof.

The invention relates likewise to the salts which the compounds of formula I are able to form with amines, alkali metal and alkaline earth metal bases and quaternary ammonium bases.

Among the alkali metal and alkaline earth metal hydroxides as salt formers, special mention should be made of the hydroxides of lithium, sodium, potassium, magnesium and calcium, but especially the hydroxides of sodium and potassium. The compounds of formula I according to the invention also include hydrates which may be formed during the salt formation.

Examples of amines suitable for ammonium salt formation include ammonia as well as primary, secondary and tertiary C₁-C₁₈alkylamines, C₁-C₄hydroxyalkylamines and C₂-C₄alkoxyalkylamines, for example methylamine, ethylamine, n-propylamine, isopropylamine, the four butylamine isomers, n-amylamine, isoamylamine, hexylamine, heptylamine, octylamine, nonylamine, decylamine, pentadecylamine, hexadecylamine, heptadecyl amine, octadecyl amine, methyl ethyl amine; methylisopropylamine, methylhexylamine, methylnonylamine, methylpentadecylamine, methyloctadecylamine, ethylbutylamine, ethylheptylamine, ethyloctylamine, hexylheptylamine, hexyloctylamine, dimethylamine, diethylamine, di-n-propylamine, diisopropylamine, di-n-butylamine, di-n-amylamine, diisoamylamine, dihexylamine, diheptylamine, dioctylamine, ethanolamine, n-propanolamine, isopropanolamine, N,N-diethanolamine, N-ethylpropanolamine, N-butylethanolamine, allylamine, n-butenyl-2-amine, n-pentenyl-2-amine, 2,3-dimethylbutenyl-2-amine, dibutenyl-2-amine, n-hexenyl-2-amine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines such as, for example, pyridine, quinoline, isoquinoline, morpholine, piperidine, pyrrolidine, indoline, quinuclidine and azepine; primary arylamines such as, for example, anilines, methoxyanilines, ethoxyanilines, o-, m- and p-toluidines, phenylenediamines, benzidines, naphthylamines and o-, m- and p-chloroanilines; but especially triethylamine, isopropylamine and diisopropylamine.

Preferred quaternary ammonium bases suitable for salt formation correspond, for example, to the formula [N(RₐR_{b}R_{c}R_{d})]OH wherein Rₐ, R_{b}, R_{c} and R_{d} are each independently of the others C₁-C₄alkyl. Other suitable tetraalkylammonium bases with other anions can be obtained, for example, by anion exchange reactions.

TABLE 1
Compounds of formula Ia

| | |
|---|---|
| | (Ia) |
| 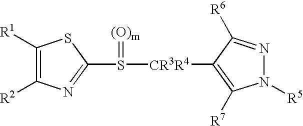 | |

| R¹ | R² | m | R³ | R⁴ | R⁵ | R⁶ | R⁷ |
|---|---|---|---|---|---|---|---|
| Br | H | 0 | H | H | Me | Me | F |
| Br | H | 1 | H | H | Me | Me | OCH₂CF₃ |
| Br | H | 0 | H | H | Me | Me | OCH₂CH₂OMe |
| F | H | 2 | H | H | Me | Me | F |
| F | H | 2 | H | H | Me | Me | OMe |
| Br | H | 1 | H | H | Me | Me | F |
| Br | H | 0 | H | H | Me | Me | OEt |
| Cl | H | 2 | H | H | Me | Me | OMe |
| Br | H | 1 | H | H | Me | Me | OCH₂CH₂OMe |
| F | H | 0 | H | H | Me | Me | F |
| Cl | H | 0 | H | H | Me | Me | OCH₂C≡CH |
| Cl | H | 1 | H | H | Me | Me | OEt |
| F | H | 0 | H | H | Me | Me | OCHF₂ |
| Cl | H | 1 | H | H | Me | Me | Cl |
| F | H | 2 | H | H | Me | Me | OCH₂C≡CH |
| F | H | 2 | H | H | Me | Me | OCH₂CF₃ |
| F | H | 1 | H | H | Me | Me | Cl |
| F | H | 2 | H | H | Me | Me | OCH₂CH₂OMe |
| Cl | H | 2 | H | H | Me | Me | OEt |
| Cl | H | 1 | H | H | Me | Me | OMe |
| Br | H | 1 | H | H | Me | Me | OEt |
| F | H | 2 | H | H | Me | Me | OEt |
| Cl | H | 2 | H | H | Me | Me | Cl |
| Cl | H | 1 | H | H | Me | Me | OCH₂C≡CH |
| Cl | H | 1 | H | H | Me | Me | OCH₂CH₂OMe |
| Cl | H | 1 | H | H | Me | Me | OCH₂CF₃ |
| Br | H | 2 | H | H | Me | Me | F |
| Br | H | 2 | H | H | Me | Me | OCH₂C≡CH |
| Br | H | 1 | H | H | Me | Me | OMe |
| Br | H | 0 | H | H | Me | Me | Cl |
| F | H | 0 | H | H | Me | Me | OCH₂CF₃ |
| Cl | H | 0 | H | H | Me | Me | OCHF₂ |
| F | H | 1 | H | H | Me | Me | OCH₂C≡CH |
| F | H | 0 | H | H | Me | Me | OCH₂C≡CH |
| Cl | H | 2 | H | H | Me | Me | F |
| Br | H | 2 | H | H | Me | Me | OMe |
| Br | H | 1 | H | H | Me | Me | OCH₂C≡CH |
| Br | H | 0 | H | H | Me | Me | OMe |
| F | H | 1 | H | H | Me | Me | OCH₂CH₂OMe |
| Cl | H | 1 | H | H | Me | Me | OCHF₂ |
| Cl | H | 2 | H | H | Me | Me | OCH₂CF₃ |
| Br | H | 2 | H | H | Me | Me | OEt |
| F | H | 1 | H | H | Me | Me | OCHF₂ |
| Br | H | 0 | H | H | Me | Me | OCH₂C≡CH |
| Br | H | 2 | H | H | Me | Me | OCH₂CH₂OMe |
| Cl | H | 0 | H | H | Me | Me | OMe |
| Cl | H | 1 | H | H | Me | Me | F |
| Cl | H | 0 | H | H | Me | Me | OCH₂CH₂OMe |
| Cl | H | 2 | H | H | Me | Me | OCH₂CH₂OMe |
| Cl | H | 0 | H | H | Me | Me | Cl |
| F | H | 0 | H | H | Me | Me | OCH₂CH₂OMe |
| Cl | H | 0 | H | H | Me | Me | OEt |
| Br | H | 2 | H | H | Me | Me | OCH₂CF₃ |
| Br | H | 0 | H | H | Me | Me | OCHF₂ |
| F | H | 1 | H | H | Me | Me | OCH₂CF₃ |
| F | H | 1 | H | H | Me | Me | OEt |
| F | H | 1 | H | H | Me | Me | OMe |
| Cl | H | 0 | H | H | Me | Me | OCH₂CF₃ |
| Br | H | 0 | H | H | Me | Me | OCH₂CF₃ |
| F | H | 2 | H | H | Me | Me | Cl |
| F | H | 1 | H | H | Me | Me | F |
| F | H | 0 | H | H | Me | Me | OMe |
| F | H | 2 | H | H | Me | Me | OCHF₂ |
| Br | H | 2 | H | H | Me | Me | OCHF₂ |
| Cl | H | 0 | H | H | Me | Me | F |
| F | H | 0 | H | H | Me | Me | OEt |
| Cl | H | 2 | H | H | Me | Me | OCHF₂ |
| F | H | 0 | H | H | Me | Me | Cl |
| Cl | H | 2 | H | H | Me | Me | OCH₂C≡CH |
| Br | H | 2 | H | H | Me | Me | Cl |
| Br | H | 1 | H | H | Me | Me | Cl |
| Br | H | 1 | H | H | Me | Me | OCHF₂ |

### Table 2:

Table 2 consists of 72 compounds of the general formula Ia, where R⁶ is trifluromethyl and R¹, R², m, R³, R⁴, R⁵ and R⁷ have the values listed in Tab;e 1 of Table 2 is the same as compound 1 of Table 1 except that in compound 1 of Table 2 R⁶ is trifluromethyl instead of methyl. Similarly, compounds 2 to 72 of Table 2 are the same as compounds 2 to 72 of Table 1, respectively, except that in the compounds of Table 2 R⁶ is trifluoromethyl instead of methyl.

### Table 3:

Table 3 consists of 72 compounds of the general formula Ia, where R⁶ is difluoromethyl and R¹, R², m, R³, R⁴, R⁵ and R⁷ have the values listed in Table 1. Thus compound 1 of Table 3 is the same as compound 1 of Table 1 except that in compound 1 of Table 3 R⁶ is difluoromethyl instead of methyl. Similarly, compounds 2 to 72 of Table 3 are the same as compounds 2 to 72 of Table 1, respectively, except that in the compounds of Table 3 R⁶ is difluoromethyl instead of methyl.

TABLE 4
Compounds of formula Ia

| | |
|---|---|
| | (Ia) |
| 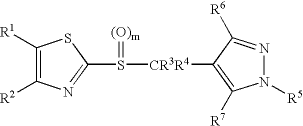 | |

| R¹ | R² | m | R³ | R⁴ | R⁵ | R⁶ | R⁷ |
|---|---|---|---|---|---|---|---|
| Cl | H | 0 | I | H | Me | Me | OEt |
| Cl | H | 2 | I | H | Me | Me | OCH₂CH₂OMe |
| Cl | H | 2 | Br | H | Me | Me | Cl |
| Cl | H | 0 | Br | H | Me | Me | F |
| Cl | H | 0 | Cl | H | Me | Me | Cl |
| Cl | H | 0 | Br | H | Me | Me | Cl |
| Cl | H | 2 | I | H | Me | Me | F |
| Cl | H | 1 | Br | H | Me | Me | OCH₂CF₃ |
| Cl | H | 0 | Cl | H | Me | Me | OCH₂C≡CH |
| Cl | H | 0 | F | H | Me | Me | OEt |
| Cl | H | 2 | Br | H | Me | Me | F |
| Cl | H | 0 | F | H | Me | Me | F |
| Cl | H | 1 | I | H | Me | Me | OCH₂CH₂OMe |
| Cl | H | 2 | F | H | Me | Me | OMe |
| Cl | H | 2 | Cl | H | Me | Me | OCHF₂ |
| Cl | H | 1 | F | H | Me | Me | OMe |
| Cl | H | 1 | Cl | H | Me | Me | OCH₂CH₂OMe |
| Cl | H | 0 | I | H | Me | Me | OMe |
| Cl | H | 0 | Br | H | Me | Me | OCH₂CF₃ |
| Cl | H | 1 | I | H | Me | Me | OCHF₂ |
| Cl | H | 0 | F | H | Me | Me | OMe |
| Cl | H | 1 | F | H | Me | Me | Cl |
| Cl | H | 2 | Cl | H | Me | Me | OEt |
| Cl | H | 0 | I | H | Me | Me | F |
| Cl | H | 1 | Cl | H | Me | Me | OCHF₂ |
| Cl | H | 2 | Br | H | Me | Me | OCH₂CF₃ |
| Cl | H | 1 | Cl | H | Me | Me | Cl |
| Cl | H | 2 | Br | H | Me | Me | OCHF₂ |
| Cl | H | 0 | Cl | H | Me | Me | OCH₂CF₃ |
| Cl | H | 1 | I | H | Me | Me | OMe |
| Cl | H | 1 | I | H | Me | Me | OCH₂C≡CH |
| Cl | H | 2 | Br | H | Me | Me | OEt |
| Cl | H | 1 | Cl | H | Me | Me | OCH₂CF₃ |
| Cl | H | 2 | F | H | Me | Me | OCH₂CF₃ |
| Cl | H | 2 | Cl | H | Me | Me | Cl |
| Cl | H | 0 | Br | H | Me | Me | OEt |
| Cl | H | 2 | Br | H | Me | Me | OMe |
| Cl | H | 1 | Br | H | Me | Me | OMe |
| Cl | H | 1 | F | H | Me | Me | OCHF₂ |
| Cl | H | 0 | F | H | Me | Me | OCH₂CH₂OMe |
| Cl | H | 0 | F | H | Me | Me | OCH₂C≡CH |
| Cl | H | 2 | F | H | Me | Me | OCHF₂ |
| Cl | H | 2 | Br | H | Me | Me | OCH₂CH₂OMe |
| Cl | H | 2 | Br | H | Me | Me | OCH₂C≡CH |
| Cl | H | 0 | F | H | Me | Me | OCHF₂ |
| Cl | H | 1 | F | H | Me | Me | OCH₂CF₃ |
| Cl | H | 1 | Cl | H | Me | Me | OEt |
| Cl | H | 0 | I | H | Me | Me | OCH₂CH₂OMe |
| Cl | H | 0 | I | H | Me | Me | OCHF₂ |
| Cl | H | 0 | I | H | Me | Me | Cl |
| Cl | H | 2 | Cl | H | Me | Me | OCH₂CH₂OMe |
| Cl | H | 1 | I | H | Me | Me | OCH₂CF₃ |
| Cl | H | 1 | F | H | Me | Me | F |
| Cl | H | 1 | Cl | H | Me | Me | OMe |
| Cl | H | 2 | F | H | Me | Me | OCH₂CH₂OMe |
| Cl | H | 2 | F | H | Me | Me | Cl |
| Cl | H | 1 | Cl | H | Me | Me | F |
| Cl | H | 0 | Cl | H | Me | Me | OCHF₂ |
| Cl | H | 0 | Cl | H | Me | Me | F |
| Cl | H | 1 | I | H | Me | Me | F |
| Cl | H | 2 | Cl | H | Me | Me | OMe |
| Cl | H | 2 | I | H | Me | Me | OEt |
| Cl | H | 2 | I | H | Me | Me | OCH₂CF₃ |
| Cl | H | 2 | F | H | Me | Me | OCH₂C≡CH |
| Cl | H | 1 | Br | H | Me | Me | OCHF₂ |
| Cl | H | 2 | Cl | H | Me | Me | F |
| Cl | H | 0 | Cl | H | Me | Me | OEt |
| Cl | H | 1 | Br | H | Me | Me | OEt |
| Cl | H | 1 | F | H | Me | Me | OCH₂C≡CH |
| Cl | H | 2 | F | H | Me | Me | OEt |
| Cl | H | 0 | Br | H | Me | Me | OCH₂C≡CH |
| Cl | H | 0 | I | H | Me | Me | OCH₂CF₃ |
| Cl | H | 1 | Br | H | Me | Me | Cl |
| Cl | H | 0 | Br | H | Me | Me | OCH₂CH₂OMe |
| Cl | H | 2 | I | H | Me | Me | OCHF₂ |
| Cl | H | 2 | Cl | H | Me | Me | OCH₂CF₃ |
| Cl | H | 1 | Br | H | Me | Me | F |
| Cl | H | 1 | Br | H | Me | Me | OCH₂CH₂OMe |
| Cl | H | 1 | I | H | Me | Me | Cl |
| Cl | H | 1 | F | H | Me | Me | OCH₂CH₂OMe |
| Cl | H | 2 | F | H | Me | Me | F |
| Cl | H | 0 | Cl | H | Me | Me | OMe |
| Cl | H | 1 | Br | H | Me | Me | OCH₂C≡CH |
| Cl | H | 2 | I | H | Me | Me | Cl |
| Cl | H | 1 | I | H | Me | Me | OEt |
| Cl | H | 0 | I | H | Me | Me | OCH₂C≡CH |
| Cl | H | 1 | Cl | H | Me | Me | OCH₂C≡CH |
| Cl | H | 2 | I | H | Me | Me | OCH₂C≡CH |
| Cl | H | 0 | Cl | H | Me | Me | OCH₂CH₂OMe |
| Cl | H | 0 | F | H | Me | Me | OCH₂CF₃ |
| Cl | H | 0 | Br | H | Me | Me | OCHF₂ |
| Cl | H | 0 | Br | H | Me | Me | OMe |
| Cl | H | 1 | F | H | Me | Me | OEt |
| Cl | H | 0 | F | H | Me | Me | Cl |
| Cl | H | 2 | I | H | Me | Me | OMe |
| Cl | H | 2 | Cl | H | Me | Me | OCH₂C≡CH |

### Table 5:

Table 5 consists of 96 compounds of the general formula Ia, where R¹ is bromo, and R², m, R³, R⁴, R⁵, R⁶ and R⁷ have the values listed in Table 4. Thus compound 1 of Table 5 is the same as compound 1 of Table 4 except that in compound 1 of Table 5 R¹ is bromo instead of chloro. Similarly, compounds 2 to 96 of Table 5 are the same as compounds 2 to 96 of Table 4, respectively, except that in the compounds of Table 5 R¹ is bromo instead of chloro.

### Table 6:

Table 6 consists of 96 compounds of the general formula Ia, where R¹ is fluoro, and R², m, R³, R⁴, R⁵, R⁶ and R⁷ have the values listed in Table 4. Thus compound 1 of Table 6 is the same as compound 1 of Table 4 except that in compound 1 of Table 6 R¹ is fluoro instead of chloro. Similarly, compounds 2 to 96 of Table 6 are the same as compounds 2 to 96 of Table 4, respectively, except that in the compounds of Table 6 R¹ is fluoro instead of chloro.

TABLE 7
Compounds of formula Ia

| | |
|---|---|
| | (Ia) |
| 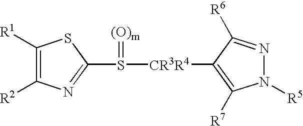 | |

| R¹ | R² | m | R³ | R⁴ | R⁵ | R⁶ | R⁷ |
|---|---|---|---|---|---|---|---|
| Cl | H | 2 | Cl | F | Me | Me | F |
| Cl | H | 1 | Cl | F | Me | Me | OCH₂C≡CH |
| Cl | H | 0 | Cl | F | Me | Me | OCHF₂ |
| Cl | H | 1 | Cl | Cl | Me | Me | OCH₂CF₃ |
| Cl | H | 1 | F | F | Me | Me | F |
| Cl | H | 0 | Cl | Cl | Me | Me | F |
| Cl | H | 1 | Cl | F | Me | Me | OMe |
| Cl | H | 2 | Cl | Cl | Me | Me | OCHF₂ |
| Cl | H | 1 | Cl | F | Me | Me | OEt |
| Cl | H | 1 | Cl | F | Me | Me | OCH₂CF₃ |
| Cl | H | 2 | Cl | F | Me | Me | OEt |
| Cl | H | 0 | F | F | Me | Me | OCH₂C≡CH |
| Cl | H | 0 | F | F | Me | Me | F |
| Cl | H | 1 | Cl | F | Me | Me | Cl |
| Cl | H | 1 | F | F | Me | Me | OCH₂CF₃ |
| Cl | H | 0 | Cl | F | Me | Me | Cl |
| Cl | H | 0 | F | F | Me | Me | OEt |
| Cl | H | 2 | Cl | F | Me | Me | Cl |
| Cl | H | 0 | F | F | Me | Me | OCH₂CH₂OMe |
| Cl | H | 0 | Cl | Cl | Me | Me | OCH₂C≡CH |
| Cl | H | 0 | Cl | Cl | Me | Me | Cl |
| Cl | H | 1 | Cl | F | Me | Me | F |
| Cl | H | 2 | F | F | Me | Me | F |
| Cl | H | 0 | Cl | Cl | Me | Me | OCHF₂ |
| Cl | H | 2 | Cl | Cl | Me | Me | OEt |
| Cl | H | 2 | F | F | Me | Me | OCH₂CH₂OMe |
| Cl | H | 0 | Cl | Cl | Me | Me | OCH₂CH₂OMe |
| Cl | H | 0 | Cl | F | Me | Me | OMe |
| Cl | H | 2 | Cl | F | Me | Me | OCH₂CF₃ |
| Cl | H | 1 | F | F | Me | Me | OCHF₂ |
| Cl | H | 2 | Cl | Cl | Me | Me | Cl |
| Cl | H | 1 | Cl | Cl | Me | Me | OCH₂CH₂OMe |
| Cl | H | 1 | F | F | Me | Me | OMe |
| Cl | H | 2 | Cl | Cl | Me | Me | OCH₂CF₃ |
| Cl | H | 2 | F | F | Me | Me | OCH₂C≡CH |
| Cl | H | 2 | F | F | Me | Me | Cl |
| Cl | H | 2 | Cl | Cl | Me | Me | OCH₂CH₂OMe |
| Cl | H | 0 | Cl | F | Me | Me | OCH₂C≡CH |
| Cl | H | 1 | F | F | Me | Me | OEt |
| Cl | H | 0 | Cl | F | Me | Me | OCH₂CH₂OMe |
| Cl | H | 2 | Cl | Cl | Me | Me | F |
| Cl | H | 2 | Cl | F | Me | Me | OCH₂CH₂OMe |
| Cl | H | 0 | Cl | F | Me | Me | OCH₂CF₃ |
| Cl | H | 1 | Cl | Cl | Me | Me | OMe |
| Cl | H | 2 | F | F | Me | Me | OEt |
| Cl | H | 2 | F | F | Me | Me | OCHF₂ |
| Cl | H | 2 | Cl | Cl | Me | Me | OMe |
| Cl | H | 0 | Cl | Cl | Me | Me | OMe |
| Cl | H | 1 | Cl | Cl | Me | Me | F |
| Cl | H | 0 | Cl | Cl | Me | Me | OCH₂CF₃ |
| Cl | H | 2 | F | F | Me | Me | OMe |
| Cl | H | 2 | Cl | F | Me | Me | OMe |
| Cl | H | 0 | F | F | Me | Me | OCHF₂ |
| Cl | H | 1 | Cl | Cl | Me | Me | Cl |
| Cl | H | 0 | Cl | F | Me | Me | OEt |
| Cl | H | 1 | F | F | Me | Me | Cl |
| Cl | H | 1 | F | F | Me | Me | OCH₂CH₂OMe |
| Cl | H | 1 | Cl | F | Me | Me | OCH₂CH₂OMe |
| Cl | H | 0 | Cl | F | Me | Me | F |
| Cl | H | 1 | Cl | Cl | Me | Me | OCHF₂ |
| Cl | H | 0 | F | F | Me | Me | Cl |
| Cl | H | 2 | F | F | Me | Me | OCH₂CF₃ |
| Cl | H | 2 | Cl | F | Me | Me | OCHF₂ |
| Cl | H | 1 | F | F | Me | Me | OCH₂C≡CH |
| Cl | H | 1 | Cl | Cl | Me | Me | OCH₂C≡CH |
| Cl | H | 0 | F | F | Me | Me | OMe |
| Cl | H | 1 | Cl | F | Me | Me | OCHF₂ |
| Cl | H | 1 | Cl | Cl | Me | Me | OEt |
| Cl | H | 0 | Cl | Cl | Me | Me | OEt |
| Cl | H | 2 | Cl | Cl | Me | Me | OCH₂C≡CH |
| Cl | H | 2 | Cl | F | Me | Me | OCH₂C≡CH |
| Cl | H | 0 | F | F | Me | Me | OCH₂CF₃ |

### Table 8:

Table 8 consists of 72 compounds of the general formula Ia, where R¹ is bromo, and R², m, R³, R⁴, R⁵, R⁶ and R⁷ have the values listed in Table 7. Thus compound 1 of Table 8 is the same as compound 1 of Table 7 except that in compound 1 of Table 8 R₁ is bromo instead of chloro. Similarly, compounds 2 to 72 of Table 8 are the same as compounds 2 to 72 of Table 7, respectively, except that in the compounds of Table 8 R¹ is bromo instead of chloro.

### Table 9:

Table 9 consists of 72 compounds of the general formula Ia, where R¹ is fluoro, and R², m, R³, R⁴, R⁵, R⁶ and R⁷ have the values listed in Table 7. Thus compound 1 of Table 9 is the same as compound 1 of Table 7 except that in compound 1 of Table 9 R is fluoro instead of chloro. Similarly, compounds 2 to 72 of Table 9 are the same as compounds 2 to 72 of Table 7, respectively, except that in the compounds of Table 9 R is fluoro instead of chloro.

### Table 10:

Table 10 consists of 96 compounds of the general formula Ia, where R⁶ is trifluoro-methyl, and R¹, R², m, R³, R^{4, R}⁵ and R⁷ have the values listed in Table 4. Thus compound 1 of Table 10 is the same as compound 1 of Table 4 except that in compound 1 of Table 10 R⁶ is trifluoromethyl instead of methyl. Similarly, compounds 2 to 96 of Table 10 are the same as compounds 2 to 96 of Table 4, respectively, except that in the compounds of Table 10 R⁶ is trifluoromethyl instead of methyl.

### Table 11:

Table 11 consists of 96 compounds of the general formula Ia, where R¹ is bromo and R⁶ is trifluoromethyl, and R², m, R³, R⁴, R⁵ and R⁷ have the values listed in Table 4. Thus compound 1 of Table 11 is the same as compound 1 of Table 4 except that in compound 1 of Table 11 R¹ is bromo instead of chloro and R⁶is trifluoromethyl instead of methyl. Similarly, compounds 2 to 96 of Table 11 are the same as compounds 2 to 96 of Table 4, respectively, except that in the compounds of Table 11 R¹ is bromo instead of chloro and

R⁶ is trifluoromethyl instead of methyl.

### Table 12:

Table 12 consists of 96 compounds of the general formula Ia, where R¹ is fluoro and R⁶ is trifluoromethyl, and R², m, R³, R⁴, R⁵ and R⁷ have the values listed in Table 4. Thus 5 compound 1 of Table 12 is the same as compound 1 of Table 4 except that in compound 1 of Table 12 R¹ is fluoro instead of chloro and R⁶ is trifluoromethyl instead of methyl.

Similarly, compounds 2 to 96 of Table 12 are the same as compounds 2 to 96 of Table 4, respectively, except that in the compounds of Table 12 R¹ is fluoro instead of chloro and R⁶ is trifluoromethyl instead of methyl.

### Table 13:

Table 13 consists of 72 compounds of the general formula Ia, where R⁶ is trifluoro-methyl, and R¹ , R², m, R³, R⁴, R⁵ and R⁷ have the values listed in Table 7. Thus compound 1 of Table 13 is the same as compound 1 of Table 7 except that in compound 1 of Table 13 R⁶is trifluoromethyl instead of methyl. Similarly, compounds 2 to 72 of 15 Table 13 are the same as compounds 2 to 72 of Table 7, respectively, except that in the compounds of Table 13 R⁶ is trifluoromethyl instead of methyl.

### Table 14:

Table 14 consists of 72 compounds of the general formula Ia, where R¹ is bromo and R⁶ is trifluoromethyl, and R², m, R³, R⁴, R⁵ and R⁷ have the values listed in Table 7. Thus 20 compound 1 of Table 14 is the same as compound 1 of Table 7 except that in compound 1 of Table 14 R¹ is bromo instead of chloro and R⁶ is trifluoromethyl instead of methyl.

Similarly, compounds 2 to 72 of Table 14 are the same as compounds 2 to 72 of Table 7, respectively, except that in the compounds of Table 14 R¹ is bromo instead of chloro and R⁶ is trifluoromethyl instead of methyl.

### Table 15:

Table 15 consists of 72 compounds of the general formula Ia, where R¹ is fluoro and R⁶ is trifluoromethyl, and R², m, R³, R⁴, R⁵ and R⁷ have the values listed in Table 7. Thus compound 1 of Table 15 is the same as compound 1 of Table 7 except that in compound 1 of Table 15 R is fluoro instead of chloro and R⁶is trifluoromethyl instead of methyl. 30 Similarly, compounds 2 to 72 of Table 15 are the same as compounds 2 to 72 of Table 7, respectively, except that in the compounds of Table 15 R¹ is fluoro instead of chloro and R⁶ is trifluoromethyl instead of methyl.

### Table 16:

Table 16 consists of 96 compounds of the general formula Ia, where R⁶is difluoro-methyl, and R¹, R², m, R³, R⁴, R⁵ and R⁷ have the values listed in Table 4. Thus compound 1 of Table 16 is the same as compound 1 of Table 4 except that in compound 1 of Table 16 R⁶is difluoromethyl instead of methyl. Similarly, compounds 2 to 96 of Table 16 are the same as compounds 2 to 96 of Table 4, respectively, except that in the compounds of Table 16 R⁶ is difluoromethyl instead of methyl.

### Table 17:

Table 17 consists of 96 compounds of the general formula Ia, where R¹ is bromo and R⁶ is difluoromethyl, and R², m, R³, R⁴, R¹ and R⁷ have the values listed in Table 4. Thus compound 1 of Table 17 is the same as compound 1 of Table 4 except that in compound 1 of Table 17 R¹ is bromo instead of chloro and R⁶is difluoromethyl instead of methyl. Similarly, compounds 2 to 96 of Table 17 are the same as compounds 2 to 96 of Table 4, respectively, except that in the compounds of Table 17 R¹ is bromo instead of chloro and R⁶ is difluoromethyl instead of methyl.

### Table 18:

Table 18 consists of 96 compounds of the general formula Ia, where R is fluoro and R⁶ is difluoromethyl, and R², m, R³, R⁴,R⁵ and R⁷ have the values listed in Table 4. Thus compound 1 of Table 18 is the same as compound 1 of Table 4 except that in compound 1 of Table 18 R¹ is fluoro instead of chloro and R⁶ is difluoromethyl instead of methyl. Similarly, compounds 2 to 96 of Table 18 are the same as compounds 2 to 96 of Table 4, respectively, except that in the compounds of Table 18 R¹ is fluoro instead of chloro and R⁶ is difluoromethyl instead of methyl.

### Table 19:

Table 19 consists of 72 compounds of the general formula Ia, where R⁶ is difluoro-methyl, and R¹ , R², m, R³,R⁴, R⁵ and R⁷ have the values listed in Table 7. Thus compound 1 of Table 19 is the same as compound 1 of Table 7 except that in compound 1 of Table 19 R⁶ is difluoromethyl instead of methyl. Similarly, compounds 2 to 72 of Table 19 are the same as compounds 2 to 72 of Table 7, respectively, except that in the compounds of Table 19 R⁶is difluoromethyl instead of methyl.

Table 20: Table 20 consists of 72 compounds of the general formula Ia, where R¹ is bromo and R⁶ is difluoromethyl, and R², m, R³, R⁴, R⁵ and R⁷ have the values listed in Table 7. Thus compound 1 of Table 20 is the same as compound 1 of Table 7 except that in compound 1 of Table 20 R¹ is bromo instead of chloro and R⁶ is difluoromethyl instead of methyl. Similarly, compounds 2 to 72 of Table 20 are the same as compounds 2 to 72 of Table 7, respectively, except that in the compounds of Table 20 R¹ is bromo instead of chloro and R⁶ is difluoromethyl instead of methyl.

### Table 21:

Table 21 consists of 72 compounds of the general formula Ia, where R¹ is fluoro and R⁶ is difluoromethyl, and R², m, R³, R⁴, R⁵ and R⁷ have the values listed in Table 7. Thus compound 1 of Table 21 is the same as compound 1 of Table 7 except that in compound 1 of Table 21 R¹ is fluoro instead of chloro and R⁶ is difluoromethyl instead of methyl. Similarly, compounds 2 to 72 of Table 21 are the same as compounds 2 to 72 of Table 7, respectively, except that in the compounds of Table 21 R¹ is fluoro instead of chloro and R⁶ is difluoromethyl instead of methyl.

TABLE 22
Compounds of formula Ia

| | |
|---|---|
| | (Ia) |
| 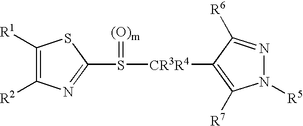 | |

| R¹ | R² | m | R³ | R⁴ | R⁵ | R⁶ | R⁷ |
|---|---|---|---|---|---|---|---|
| Cl | H | 0 | Me | F | Me | Me | OEt |
| Cl | H | 1 | Me | H | Me | Me | OCH₂C≡CH |
| Cl | H | 0 | Me | Me | Me | Me | Cl |
| Cl | H | 0 | Me | H | Me | Me | F |
| Cl | H | 0 | Me | H | Me | Me | OCHF₂ |
| Cl | H | 0 | Me | Me | Me | Me | OCH₂C≡CH |
| Cl | H | 2 | Me | Me | Me | Me | OCH₂CF₃ |
| Cl | H | 2 | Me | Me | Me | Me | OMe |
| Cl | H | 1 | Me | F | Me | Me | OCH₂CF₃ |
| Cl | H | 0 | Me | F | Me | Me | OMe |
| Cl | H | 2 | Me | F | Me | Me | OEt |
| Cl | H | 0 | Me | Me | Me | Me | OCH₂CF₃ |
| Cl | H | 2 | Me | F | Me | Me | OMe |
| Cl | H | 2 | Me | Me | Me | Me | OEt |
| Cl | H | 2 | Me | F | Me | Me | OCHF₂ |
| Cl | H | 1 | Me | F | Me | Me | F |
| Cl | H | 1 | Me | H | Me | Me | OEt |
| Cl | H | 2 | Me | F | Me | Me | OCH₂CH₂OMe |
| Cl | H | 2 | Me | H | Me | Me | F |
| Cl | H | 1 | Me | Me | Me | Me | OCH₂CF₃ |
| Cl | H | 0 | Me | F | Me | Me | OCH₂C≡CH |
| Cl | H | 1 | Me | H | Me | Me | Cl |
| Cl | H | 0 | Me | H | Me | Me | OEt |
| Cl | H | 1 | Me | F | Me | Me | OEt |
| Cl | H | 0 | Me | Me | Me | Me | OCHF₂ |
| Cl | H | 0 | Me | F | Me | Me | Cl |
| Cl | H | 2 | Me | F | Me | Me | Cl |
| Cl | H | 1 | Me | F | Me | Me | Cl |
| Cl | H | 2 | Me | H | Me | Me | OMe |
| Cl | H | 2 | Me | Me | Me | Me | F |
| Cl | H | 0 | Me | Me | Me | Me | F |
| Cl | H | 0 | Me | F | Me | Me | OCH₂CH₂OMe |
| Cl | H | 0 | Me | H | Me | Me | OCH₂CF₃ |
| Cl | H | 1 | Me | H | Me | Me | OCH₂CF₃ |
| Cl | H | 0 | Me | H | Me | Me | OMe |
| Cl | H | 1 | Me | Me | Me | Me | OEt |
| Cl | H | 2 | Me | Me | Me | Me | OCH₂C≡CH |
| Cl | H | 2 | Me | H | Me | Me | OCH₂CH₂OMe |
| Cl | H | 2 | Me | Me | Me | Me | Cl |
| Cl | H | 2 | Me | Me | Me | Me | OCHF₂ |
| Cl | H | 2 | Me | H | Me | Me | OCHF₂ |
| Cl | H | 0 | Me | H | Me | Me | OCH₂C≡CH |
| Cl | H | 0 | Me | F | Me | Me | OCH₂CF₃ |
| Cl | H | 0 | Me | Me | Me | Me | OCH₂CH₂OMe |
| Cl | H | 0 | Me | Me | Me | Me | OMe |
| Cl | H | 1 | Me | Me | Me | Me | OCH₂C≡CH |
| Cl | H | 1 | Me | H | Me | Me | OCH₂CH₂OMe |
| Cl | H | 1 | Me | Me | Me | Me | OMe |
| Cl | H | 0 | Me | H | Me | Me | Cl |
| Cl | H | 2 | Me | H | Me | Me | OCH₂CF₃ |
| Cl | H | 2 | Me | H | Me | Me | Cl |
| Cl | H | 2 | Me | H | Me | Me | OCH₂C≡CH |
| Cl | H | 1 | Me | Me | Me | Me | OCHF₂ |
| Cl | H | 1 | Me | F | Me | Me | OCHF₂ |
| Cl | H | 1 | Me | F | Me | Me | OMe |
| Cl | H | 2 | Me | F | Me | Me | F |
| Cl | H | 1 | Me | F | Me | Me | OCH₂C≡CH |
| Cl | H | 0 | Me | H | Me | Me | OCH₂CH₂OMe |
| Cl | H | 2 | Me | F | Me | Me | OCH₂C≡CH |
| Cl | H | 1 | Me | H | Me | Me | OCHF₂ |
| Cl | H | 1 | Me | H | Me | Me | F |
| Cl | H | 0 | Me | Me | Me | Me | OEt |
| Cl | H | 1 | Me | Me | Me | Me | Cl |
| Cl | H | 2 | Me | H | Me | Me | OEt |
| Cl | H | 1 | Me | Me | Me | Me | F |
| Cl | H | 1 | Me | H | Me | Me | OMe |
| Cl | H | 1 | Me | F | Me | Me | OCH₂CH₂OMe |
| Cl | H | 0 | Me | F | Me | Me | OCHF₂ |
| Cl | H | 1 | Me | Me | Me | Me | OCH₂CH₂OMe |
| Cl | H | 2 | Me | Me | Me | Me | OCH₂CH₂OMe |
| Cl | H | 2 | Me | F | Me | Me | OCH₂CF₃ |
| Cl | H | 0 | Me | F | Me | Me | F |

### Table 23:

Table 23 consists of 72 compounds of the general formula Ia, where R¹ is bromo, and R², m, R³, R⁴, R⁵, R⁶ and R⁷ have the values listed in Table 22. Thus compound 1 of Table 23 is the same as compound 1 of Table 22 except that in compound 1 of Table 23 R is bromo instead of chloro. Similarly, compounds 2 to 72 of Table 23 are the same as compounds 2 to 72 of Table 22, respectively, except that in the compounds of Table 23 R¹ is bromo instead of chloro.

### Table 24:

Table 24 consists of 72 compounds of the general formula Ia, where R¹ is fluoro, and R², m, R³, R⁴, R⁵, R⁶ and R⁷ have the values listed in Table 22. Thus compound 1 of Table 24 is the same as compound 1 of Table 22 except that in compound 1 of Table 24 R¹ is fluoro instead of chloro. Similarly, compounds 2 to 72 of Table 24 are the same as compounds 2 to 72 of Table 22, respectively, except that in the compounds of Table 24 R¹ is fluoro instead of chloro.

### Table 25:

Table 25 consists of 72 compounds of the general formula Ia, where R⁶ is trifluoro-methyl, and R¹, R², m, R³, R⁴, R⁵ and R⁷ have the values listed in Table 22. Thus compound 1 of Table 25 is the same as compound 1 of Table 22 except that in compound 1 of Table 25 R⁶ is trifluoromethyl instead of methyl. Similarly, compounds 2 to 72 of Table 25 are the same as compounds 2 to 72 of Table 22, respectively, except that in the compounds of Table 25 R⁶ is trifluoromethyl instead of methyl.

### Table 26:

Table 26 consists of 72 compounds of the general formula Ia, where R¹ is bromo and R⁶ is trifluoromethyl, and R², m, R³, R⁴, R⁵ and R⁷ have the values listed in Table 22. Thus compound 1 of Table 26 is the same as compound 1 of Table 22 except that in compound 1 of Table 26 R¹ is bromo instead of chloro and R⁶ is trifluoromethyl instead of methyl. Similarly, compounds 2 to 72 of Table 26 are the same as compounds 2 to 72 of Table 22, respectively, except that in the compounds of Table 26 R¹ is bromo instead of chloro and R⁶ is trifluoromethyl instead of methyl.

### Table 27:

Table 27 consists of 72 compounds of the general formula Ia, where R¹ is fluoro and R⁶ is trifluoromethyl, and R², m, R³, R⁴, R⁵ and R⁷ have the values listed in Table 22. Thus compound 1 of Table 27 is the same as compound 1 of Table 22 except that in compound 1 of Table 27 R¹ is fluoro instead of chloro and R⁶ is trifluoromethyl instead of methyl. Similarly, compounds 2 to 72 of Table 27 are the same as compounds 2 to 72 of Table 22, respectively, except that in the compounds of Table 27 R¹ is fluoro instead of chloro and R⁶ is trifluoromethyl instead of methyl.

### Table 28:

Table 28 consists of 72 compounds of the general formula Ia, where R⁶ is difluoro-methyl, and R¹ , R², m, R³, R⁴, R⁵ and R⁷ have the values listed in Table 22. Thus compound 1 of Table 28 is the same as compound 1 of Table 22 except that in compound 1 of Table 28 R⁶ is difluoromethyl instead of methyl. Similarly, compounds 2 to 72 of Table 28 are the same as compounds 2 to 72 of Table 22, respectively, except that in the compounds of Table 28 R⁶ is difluoromethyl instead of methyl.

### Table 29:

Table 29 consists of 72 compounds of the general formula Ia, where R¹ is bromo and R⁶ is difluoromethyl, and R², m, R³, R⁴, R₅ and R⁷ have the values listed in Table 22. Thus compound 1 of Table 29 is the same as compound 1 of Table 22 except that in compound 1 of Table 29 R¹ is bromo instead of chloro and R⁶ is difluoromethyl instead of methyl. Similarly, compounds 2 to 72 of Table 29 are the same as compounds 2 to 72 of Table 22, respectively, except that in the compounds of Table 29 R¹ is bromo instead of chloro and R⁶ is difluoromethyl instead of methyl.

### Table 31:

Table 31 consists of 72 compounds of the general formula Ia, where R¹ is fluoro and R⁶ is difluoromethyl, and R², m, R³, R⁴, R⁵ and R⁷ have the values listed in Table 23. Thus compound 1 of Table 31 is the same as compound 1 of Table 23 except that in compound 1 of Table 31 R¹ is fluoro instead of chloro and R⁶ is difluoromethyl instead of methyl. Similarly, compounds 2 to 72 of Table 31 are the same as compounds 2 to 72 of Table 23, respectively, except that in the compounds of Table 31 R¹ is fluoro instead of chloro and R⁶ is difluoromethyl instead of methyl.

Preferably R¹ is hydrogen, C₁-C₆alkyl, C₃-C₆cycloalkyl, C₁-C₆haloalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, nitro, cyano, formyl, halogen, tri(C₁-C₆alkyl)silyl, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, C₁-C₆alkoxy, C₁-C₆haloalkoxy, —NHSO₂—C₁-C₆alkyl, —NHSO₂—C₁-C₆haloalkyl, —NHCO—C₁-C₆alkyl, —NHCO—C₁-C₆haloalkyl, —NHCO₂—C₁-C₆alkyl, —O(CO)—C₁-C₆alkyl, —O(CO)—C₁-C₆haloalkyl, —OCONH—C₁-C₆alkyl, —OCONH—C₁-C₆haloalkyl, or —CONR^{a}R^{b} wherein R^{a} and R^{b} are each independently of the other hydrogen, C₁-C₆alkyl or C₃-C₆cycloalkyl, more preferably R¹ is hydrogen, methyl, ethyl, cyclopropyl, difluoromethyl, trifluoromethyl, vinyl, ethynyl, acetyl, trifluoroacetyl, methoxycarbonyl, ethoxycarbonyl, nitro, cyano, formyl, bromo, chloro, fluoro, trimethylsilyl, methylthio, methylsulfinyl, methylsulfonyl, trifluoromethylthio, trifluoromethylsulfinyl, trifluoromethylsulfonyl, methoxy, difluoromethoxy, trifluoromethoxy, —NHSO₂Me, —NHSO₂CF₃, —NHCOMe, —NHCOCF₃, —NHCO₂Me, —O(CO)Me, —O(CO)CF₃, —O(CO)NHMe, —CONH^{t}Bu, —CONH^{c}Pr or —CONH₂, even more preferably R¹ is hydrogen, methyl, difluoromethyl, acetyl, trifluoroacetyl, methoxycarbonyl, ethoxycarbonyl, nitro, cyano, bromo, chloro, trimethylsilyl, —CONH^{t}Bu, —CONH^{c}Pr or —CONH₂, most preferably R¹ is hydrogen, methyl, difluoromethyl, nitro, bromo or chloro.

Preferably R² is hydrogen, C₁-C₆alkyl, C₃-C₆cycloalkyl, C₁-C₆haloalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, nitro, cyano, formyl, halogen, tri(C₁-C₆alkyl)silyl, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, C₁-C₆alkoxy, C₁-C₆haloalkoxy, —NHSO₂—C₁-C₆alkyl, —NHSO₂—C₁-C₆haloalkyl, —NHCO—C₁-C₆alkyl, —NHCO—C₁-C₆haloalkyl, —NHCO₂—C₁-C₆alkyl, —O(CO)—C₁-C₆alkyl, —O(CO)—C₁-C₆haloalkyl, —OCONH—C₁-C₆alkyl, —OCONH—C₁-C₆haloalkyl, or —CONR^{a}R^{b} wherein R^{a} and R^{b} are each independently of the other hydrogen, C₁-C₆alkyl or C₃-C₆cycloalkyl, more preferably R² is hydrogen, methyl, ethyl, cyclopropyl, difluoromethyl, trifluoromethyl, vinyl, ethynyl, acetyl, trifluoroacetyl, methoxycarbonyl, ethoxycarbonyl, nitro, cyano, formyl, bromo, chloro, fluoro, trimethylsilyl, methylthio, methylsulfinyl, methylsulfonyl, trifluoromethylthio, trifluoromethylsulfinyl, trifluoromethylsulfonyl, methoxy, difluoromethoxy, trifluoromethoxy, —NHSO₂Me, —NHSO₂CF₃, —NHCOMe, —NHCOCF₃, —NHCO₂Me, —O(CO)Me, —O(CO)CF₃, —O(CO)NHMe, —CONH^{t}Bu, —CONH^{c}Pr or —CONH₂, even more preferably R² is hydrogen, trifluoromethyl or bromo, most preferably R² is hydrogen or bromo.

Preferably R³ is hydrogen, C₁-C₆alkyl, halogen or C₁-C₆alkoxycarbonyl, more preferably R³ is hydrogen, methyl, ethyl, fluoro, chloro or methoxycarbonyl, most preferably R³ is hydrogen, methyl, fluoro or chloro.

Preferably R⁴ is hydrogen, C₁-C₆alkyl, halogen or C₁-C₆alkoxycarbonyl, more preferably R⁴ is hydrogen, methyl, ethyl, fluoro, chloro or methoxycarbonyl, even more preferably R⁴ is hydrogen, methyl or fluoro, most preferably R⁴ is hydrogen or fluoro.

Preferably R⁵ is hydrogen, C₁-C₁₀alkyl, C₃-C₈cycloalkyl-C₁-C₁₀alkyl, C₁-C₆alkylcarbonyl-C₁-C₁₀alkyl, C₁-C₄haloalkylcarbonyl-C₁-C₁₀alkyl, C₁-C₁₀alkoxy-C₁-C₁₀alkyl, C₁-C₄alkoxycarbonyl-C₁-C₁₀alkyl, cyano-C₁-C₁₀alkyl, C₁-C₄haloalkyl, C₃-C₈cycloalkyl, C₂-C₆alkenyl, C₁-C₄haloalkenyl, C₂-C₆alkynyl, C₁-C₁₀alkylsulfonyl, C₁-C₄haloalkylsulfonyl, C₁-C₆alkylcarbonyl, C₁-C₄haloalkylcarbonyl, C₃-C₈cycloalkylcarbonyl, C₁-C₁₀alkoxycarbonyl, or —CONR^{c}R^{d} wherein R^{c} and R^{d} are each independently of the other hydrogen or C₁-C₁₀alkyl, more preferably R⁵ is hydrogen, methyl, ethyl, cyclopropylmethyl, acetylmethyl, trifluoroacetylmethyl, methoxymethyl, 2-methoxy-ethyl, methoxycarbonylmethyl, cyanomethyl, difluoromethyl, trifluoromethyl, 2,2-difluoroethyl, 2,2,2-trifluoroethyl, cyclopropyl, vinyl, propargyl, methylsulfonyl, acetyl, trifluoroacetyl, cyclopropylcarbonyl, methoxycarbonyl, ethoxycarbonyl, —CONH^{t}Bu or —CONH₂, most preferably R⁵ is methyl.

Preferably R⁶ is hydrogen, halogen, C₁-C₁₀alkyl, C₃-C₈cycloalkyl-C₁-C₁₀alkyl, C₁-C₆alkylcarbonyl-C₁-C₁₀alkyl, C₁-C₄haloalkylcarbonyl-C₁-C₁₀alkyl, C₁-C₁₀alkoxy-C₁-C₁₀alkyl, C₁-C₄alkoxycarbonyl-C₁-C₁₀alkyl, cyano-C₁-C₁₀alkyl, C₁-C₄haloalkyl, C₃-C₈cycloalkyl, C₁-C₁₀alkoxy, C₁-C₁₀alkoxy-C₁-C₁₀alkoxy, C₁-C₁₀alkoxycarbonyl-C₁-C₁₀alkoxy, cyano-C₁-C₁₀alkoxy, C₁-C₄haloalkoxy, C₃-C₈cycloalkyloxy, C₃-C₈cycloalkyl-C₁-C₃alkoxy, C₁-C₁₀alkylthio, C₁-C₄haloalkylthio, C₂-C₆alkenyl, C₁-C₄haloalkenyl, C₂-C₆alkenyloxy, C₂-C₆alkynyl, C₂-C₆alkynyloxy, C₁-C₁₀alkylsulfinyl, C₁-C₁₀alkylsulfonyl, C₁-C₄haloalkylsulfinyl, C₁-C₄haloalkylsulfonyl, C₁-C₆alkylcarbonyl, C₁-C₄haloalkylcarbonyl, C₃-C₈cycloalkylcarbonyl, C₁-C₁₀alkoxycarbonyl, cyano, —CONR^{c}R^{d} (wherein R^{c} and R^{d} are each independently of the other hydrogen or C₁-C₁₀alkyl), nitro, or —NR^{c}R^{d} (wherein R^{c} and R^{d} are each independently of the other hydrogen, C₁-C₁₀alkyl, C₁-C₆alkylcarbonyl, C₁-C₄haloalkylcarbonyl, C₁-C₁₀alkylsulfonyl or C₁-C₄haloalkylsulfonyl), more preferably R⁶ is hydrogen, bromo, chloro, fluoro, methyl, ethyl, cyclopropylmethyl, acetylmethyl, trifluoroacetylmethyl, methoxymethyl, 2-methoxy-ethyl, methoxycarbonylmethyl, cyanomethyl, monofluoromethyl, difluoromethyl, trifluoromethyl, 2,2-difluoroethyl, 2,2,2-trifluoroethyl, cyclopropyl, C₁-C₄alkoxy, 2-methoxy-ethoxy, C₁-C₄haloalkoxy, cyclopropyloxy, cyclopropylmethoxy, C₁-C₄alkylthio, vinyl, prop-2-enyl, prop-2-enyloxy, 2-fluoroprop-2-enyloxy, propargyl, propargyloxy, C₁-C₄alkylsulfinyl, C₁-C₄alkylsulfonyl, acetyl, trifluoroacetyl, cyclopropylcarbonyl, methoxycarbonyl, ethoxycarbonyl, cyano, —CONH^{t}Bu, —CONH₂, nitro, —NH^{t}Bu or —NH₂, even more preferably R⁶ is methyl, monofluoromethyl, difluoromethyl, trifluoromethyl, methoxy, ethoxy, difluoromethoxy, trifluoromethoxy, or 2,2,2-trifluoroethoxy, most preferably R⁶ is trifluoromethyl or difluoromethoxy.

Preferably R⁷ is hydrogen, halogen, C₁-C₁₀alkyl, C₃-C₈cycloalkyl-C₁-C₁₀alkyl, C₁-C₆alkylcarbonyl-C₁-C₁₀alkyl, C₁-C₄haloalkylcarbonyl-C₁-C₁₀alkyl, C₁-C₄alkoxycarbonyl-C₁-C₁₀alkyl, cyano-C₁-C₁₀alkyl, C₁-C₄haloalkyl, C₃-C₈cycloalkyl, C₁-C₁₀alkoxy, C₁-C₁₀alkoxy-C₁-C₁₀alkoxy, C₁-C₁₀alkoxycarbonyl-C₁-C₁₀alkoxy, cyano-C₁-C₁₀alkoxy, C₁-C₄haloalkoxy, C₃-C₈cycloalkyloxy, C₃-C₈cycloalkylC₁-C₃alkoxy, C₁-C₁₀alkylthio, C₁-C₄haloalkylthio, C₂-C₆alkenyl, C₁-C₄haloalkenyl, C₂-C₆alkenyloxy, C₂-C₆alkynyl, C₂-C₆alkynyloxy, C₁-C₁₀alkylsulfinyl, C₁-C₁₀alkylsulfonyl, C₁-C₄haloalkylsulfinyl, C₁-C₄haloalkylsulfonyl, C₁-C₆alkylcarbonyl, C₁-C₄haloalkylcarbonyl, C₁-C₁₀alkoxycarbonyl, cyano, —CONR^{c}R^{d} (wherein R^{c} and R^{d} are each independently of the other hydrogen or C₁-C₁₀alkyl), nitro, or —NR^{c}R^{d} (wherein R^{c} and R^{d} are each independently of the other hydrogen, C₁-C₁₀alkyl, C₁-C₆alkylcarbonyl, C₁-C₄haloalkylcarbonyl, C₁-C₁₀alkylsulfonyl or C₁-C₄haloalkylsulfonyl), more preferably R⁷ is hydrogen, bromo, chloro, fluoro, methyl, ethyl, cyclopropylmethyl, acetylmethyl, trifluoroacetylmethyl, methoxymethyl, cyanomethyl, difluoromethyl, trifluoromethyl, cyclopropyl, C₁-C₄alkoxy, 2-methoxyethoxy, C₁-C₄haloalkoxy, cycloalkyloxy, cycloalkylmethoxy, C₁-C₄alkylthio, vinyl, prop-2-enyl, propargyloxy, prop-2-enyloxy, 2-fluoroprop-2-enyloxy, ethynyl, C₁-C₄alkylsulfinyl, C₁-C₄alkylsulfonyl, acetyl, trifluoroacetyl, methoxycarbonyl, ethoxycarbonyl, cyano, —CONH^{t}Bu, —CONH₂, nitro, —NH^{t}Bu or —NH₂, even more preferably R⁷ is hydrogen, chloro, fluoro, methyl, ethyl, trifluoromethyl, C₁-C₃alkoxy, 2-methoxyethoxy, C₁-C₃haloalkoxy, C₁-C₃alkylthio, vinyl, prop-2-enyl, propargyloxy, prop-2-enyloxy, 2-fluoroprop-2-enyloxy, ethynyl, C₁-C₃alkylsulfinyl, C₁-C₃alkylsulfonyl or cyano, most preferably R⁷ is hydrogen, chloro, fluoro, trifluoromethyl, ethoxy, 2-methoxyethoxy, difluoromethoxy, 2,2-difluoroethoxy, 2,2,2-trifluoroethoxy, 3-fluoropropyloxy, 2,2,3,3-tetrafluoropropyloxy, 1-fluoroprop-2-yloxy, 1,3-difluoroprop-2-yloxy, 1,1,1-trifluoroprop-2-yloxy, methylthio, ethylthio, 2-fluoroprop-2-enyloxy, methylsulfinyl, ethylsulfinyl, methylsulfonyl or ethylsulfonyl.

A preferred group of compounds of formula I comprises those wherein m, R³, R⁴, n, R⁵, R⁶ and R⁷ are defined as above and

R¹ and R² are each independently of the other hydrogen, C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCHO, —NHCO—C₁-C₆alkyl, —NHCO—C₁-C₆haloalkyl, —NHCO₂—C₁-C₆alkyl, —NHCO₂—C₁-C₆haloalkyl, —NHCONH—C₁-C₆alkyl, —NHCONH—C₁-C₆haloalkyl, —NHSO₂—C₁-C₆alkyl, —NHSO₂—C₁-C₆haloalkyl, —NHSO₂-phenyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR^{a}R^{b} wherein R^{a} and R^{b} are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R^{a} and R^{b} together form a C₃-C₈alkylene group which optionally contains one oxygen or sulfur atom or one or two amino or C₁-C₆alkylamino groups; and to N-oxides, salts and optical isomers of compounds of formula I.

A further preferred group of compounds of formula I comprises those wherein m, R³, R⁴, n, R⁵, R⁶ and R⁷ are defined as above and

R¹ and R² are each independently of the other hydrogen, C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR^{a}R^{b} wherein R^{a} and R^{b} are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R^{a} and R^{b} together form a C₃-C₈alkylene group which optionally contains one oxygen or sulfur atom or one or two amino or C₁-C₆alkylamino groups; and to N-oxides, salts and optical isomers of compounds of formula I.

A group of particularly preferred compounds of formula I comprises those wherein

R¹ and R² are each independently of the other hydrogen, C₁-C₆haloalkyl, C₁-C₆alkoxycarbonyl or halogen;
R³ and R⁴ are each independently of the other hydrogen, C₁-C₆alkyl or halogen;
m is 0, 1 or 2;
n is 1;
R⁵, R⁶ and R⁷ are each independently of the others halogen, C₁-C₁₀alkyl, C₁-C₄haloalkyl, C₁-C₁₀alkoxy, C₁-C₁₀alkoxyC₁-C₁₀alkoxy, C₁-C₄haloalkoxy or C₂-C₆alkynyloxy;
and to N-oxides, salts and optical isomers of compounds of formula I.

A further group of preferred compounds of formula I comprises those wherein

R¹ and R² are each independently of the other hydrogen, C₁-C₆haloalkyl, C₁-C₆alkoxycarbonyl or halogen;
R³ and R⁴ are each independently of the other hydrogen or halogen;
m is 0, 1 or 2;
n is 1;
R⁵, R⁶ and R⁷ are each independently of the others halogen, C₁-C₁₀alkyl, C₁-C₄haloalkyl, C₁-C₁₀alkoxy, C₁-C₁₀alkoxyC₁-C₁₀alkoxy, C₁-C₄haloalkoxy or C₂-C₆alkynyloxy;
and to N-oxides, salts and optical isomers of compounds of formula I.

A group of further preferred compound of formula I comprises those wherein

R¹ and R² are each independently of the other hydrogen, C₁-C₆haloalkyl, C₁-C₆alkoxycarbonyl or halogen;
R³ and R⁴ are both hydrogen;
m is 0, 1 or 2;
n is 1;
R⁵, R⁶ and R⁷ are each independently of the others halogen, C₁-C₁₀alkyl, C₁-C₄haloalkyl or C₁-C₄haloalkoxy;
and to N-oxides, salts and optical isomers of compounds of formula I.

A further group of especially preferred compounds of formula I comprises those wherein R², m, R³, R⁴, n, R⁵, R⁶ and R⁷ are as defined above and R¹ is hydrogen.

A further group of especially preferred compounds of formula I comprises those wherein R², m, R³, R⁴, n, R⁵, R⁶ and R⁷ are as defined above and R¹ is C₁-C₆alkyl, especially methyl.

A further group of especially preferred compounds of formula I comprises those wherein R², m, R³, R⁴, n, R⁵, R⁶ and R⁷ are as defined above and R¹ is C₁-C₆alkoxycarbonyl, especially ethoxycarbonyl.

A further group of especially preferred compounds of formula I comprises those wherein R², m, R³, R⁴, n, R⁵, R⁶ and R⁷ are as defined above and R¹ is halogen, especially chloro and bromo.

A further group of especially preferred compounds of formula I comprises those wherein R², m, R³, R⁴, n, R⁵, R⁶ and R⁷ are as defined above and R¹ is nitro.

A further group of especially preferred compounds of formula I comprises those wherein R¹, m, R³, R⁴, n, R⁵, R⁶ and R⁷ are as defined above and R² is hydrogen.

A further group of especially preferred compounds of formula I comprises those wherein R¹, m, R³, R⁴, n, R⁵, R⁶ and R⁷ are as defined above and R² is C₁-C₆haloalkyl, especially trifluoromethyl.

A further group of especially preferred compounds of formula I comprises those wherein R¹, m, R³, R⁴, n, R⁵, R⁶ and R⁷ are as defined above and R² is halogen, especially bromo.

A further group of especially preferred compounds of formula I comprises those wherein m is 1 or 2.

A further group of very especially preferred compounds of formula I comprises those wherein m is 1.

A further group of very especially preferred compounds of formula I comprises those wherein m is 2.

A further group of especially preferred compounds of formula I comprises those wherein R¹, R², m, n, R⁵, R⁶ and R⁷ are as defined above and R³ and R⁴ are both hydrogen.

A further group of especially preferred compounds of formula I comprises those wherein R¹, R², m, R⁴, n, R⁵, R⁶ and R⁷ are as defined above and R³ is halogen, especially fluoro or chloro.

A further group of especially preferred compounds of formula I comprises those wherein R¹, R², m, n, R⁵, R⁶ and R⁷ are as defined above and R³ is halogen, especially fluoro or chloro, and R⁴ is hydrogen.

A further group of especially preferred compounds of formula I comprises those wherein R¹, R², m, n, R⁵, R⁶ and R⁷ are as defined above and R³ is halogen, especially fluoro or chloro, and R⁴ is C₁-C₆alkyl, especially methyl.

A further group of especially preferred compounds of formula I comprises those wherein R¹, R², m, n, R⁵, R⁶ and R⁷ are as defined above and R³ and R⁴ are both halogen, especially where R³ is fluoro and R⁴ is chloro or where R³ and R⁴ are both fluoro.

A further group of especially preferred compounds of formula I comprises those wherein R¹, R², m, R⁴, n, R⁵, R⁶ and R⁷ are as defined above and R³ is C₁-C₆alkyl, especially methyl.

A further group of especially preferred compounds of formula I comprises those wherein R¹, R², m, n, R⁵, R⁶ and R⁷ are as defined above and R³ is C₁-C₆alkyl, especially methyl, and R⁴ is hydrogen.

A further group of especially preferred compounds of formula I comprises those wherein n is 1 or 2.

A further group of very especially preferred compounds of formula I comprises those wherein n is 1.

A further group of especially preferred compounds of formula I comprises those wherein R¹, R², m, R³, R⁴, n, R⁶ and R⁷ are as defined above and R⁵ is C₁-C₁₀alkyl, especially methyl.

A further group of especially preferred compounds of formula I comprises those wherein R¹, R², m, R³, R⁴, n, R⁵ and R⁷ are as defined above and R⁶ is C₁-C₁₀alkyl, especially methyl.

A further group of especially preferred compounds of formula I comprises those wherein R¹, R², m, R³, R⁴, n, R⁵ and R⁷ are as defined above and R⁶ is C₁-C₄haloalkyl, especially trifluoromethyl and difluoromethyl; most preferably trifluoromethyl.

A further group of especially preferred compounds of formula I comprises those wherein R¹, R², m, R³, R⁴, n, R⁵ and R⁷ are as defined above and R⁶ is C₁-C₄haloalkoxy, especially 2,2,2-trifluoroethoxy and difluoromethoxy; most preferably difluoromethoxy.

A further group of especially preferred compounds of formula I comprises those wherein R¹, R², m, R³, R⁴, n, R⁵ and R⁶ are as defined above and R⁷ is hydrogen.

A further group of especially preferred compounds of formula I comprises those wherein R¹, R², m, R³, R⁴, n, R⁵ and R⁶ are as defined above and R⁷ is halogen, especially fluoro and chloro.

A further group of especially preferred compounds of formula I comprises those wherein R¹, R², m, R³, R⁴, n, R⁵ and R⁶ are as defined above and R⁷ is C₁-C₄haloalkyl, especially trifluoromethyl and difluoromethyl; most preferably trifluoromethyl.

A further group of especially preferred compounds of formula I comprises those wherein R¹, R², m, R³, R⁴, n, R⁵ and R⁶ are as defined above and R⁷ is C₁-C₁₀alkoxy, especially ethoxy and methoxy.

A further group of especially preferred compounds of formula I comprises those wherein R¹, R², m, R³, R⁴, n, R⁵ and R⁶ are as defined above and R⁷ is C₁-C₁₀alkoxyC₁-C₁₀alkoxy, especially 2-methoxyethoxy.

A further group of especially preferred compounds of formula I comprises those wherein R¹, R², m, R³, R⁴, n, R⁵ and R⁶ are as defined above and R⁷ is C₁-C₄haloalkoxy, especially difluoromethoxy, 2,2-difluoroethoxy, 2,2,2-trifluoroethoxy, 3-fluoropropyloxy, 2,2,3,3-tetrafluoropropyloxy, 1-fluoroprop-2-yloxy, 1,3-difluoroprop-2-yloxy and 1,1,1-trifluoroprop-2-yloxy; most preferably 2,2,2-trifluoroethoxy and difluoromethoxy.

A further group of especially preferred compounds of formula I comprises those wherein R¹, R², m, R³, R⁴, n, R⁵ and R⁶ are as defined above and R⁷ is C₁-C₁₀alkylthio, especially C₁-C₄alkylthio; most preferably C₁-C₂alkylthio.

A further group of especially preferred compounds of formula I comprises those wherein R¹, R², m, R³, R⁴, n, R⁵ and R⁶ are as defined above and R⁷ is C₁-C₁₀alkylsulfinyl, especially C₁-C₄alkylsulfinyl; most preferably C₁-C₂alkylsulfinyl.

A further group of especially preferred compounds of formula I comprises those wherein R¹, R², m, R³, R⁴, n, R⁵ and R⁶ are as defined above and R⁷ is C₁-C₁₀alkylsulfonyl, especially C₁-C₄alkylsulfonyl; most preferably C₁-C₂alkylsulfonyl.

A further group of especially preferred compounds of formula I comprises those wherein R¹, R², m, R³, R⁴, n, R⁵ and R⁶ are as defined above and R⁷ is C₂-C₆alkynyloxy, especially prop-2-ynyloxy.

1) The compounds of formula I wherein R¹, R², R³, R⁴, R⁵, R⁶ and R⁷ are as defined above, m is 1 or 2, and n is 1, can be prepared by processes known per se, by reacting e.g. the compounds of formula Ib wherein R¹, R², R⁵, R⁶ and R⁷ are as defined as above, and m is 1 or 2,

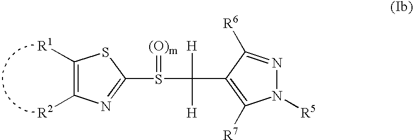

in a single step or stepwise in succession with a compound of formula R³—X and/or a compound of formula R⁴—X, wherein R³ and R⁴ are as defined above and X is a suitable leaving group e.g. halogen, such as bromide or iodide, a carboxylate, such as acetate, an alkyl-, aryl- or haloalkylsulfonate, such as methylsulfonate, p-toluenesulfonate or trifluoromethylsulfonate, an imide, such as succinimide, a sulfonimide, such as bis(phenylsulfonyl)imide, in the presence of a base, e.g. an alkyl-lithium compound, such as methyl-lithium, n-butyl-lithium or tert-butyl-lithium, a lithium dialkylamide, such as lithium diisopropylamide, a metal hydride, preferably an alkali metal hydride, such as sodium hydride, or an alkali metal amide, such as sodium amide, a metal bis(tri(C₁-C₆alkyl)silyl)amide, such as lithium bis(trimethylsilyl)amide, a metal alkoxide, such as potassium tert-butoxide, or a phosphazene base, such as N′-tert-butyl-N,N,N′,N′,N″,N″-hexamethylphosphorimidic triamide (P₁-t-Bu), 1-tert-butyl-2,2,4,4,4-pentakis(dimethylamino)-2-lambda⁵,4lambda⁵-catenadi(phosphazene) (P₂-t-Bu), 1-ethyl-2,2,4,4,4-pentakis-(dimethylamino)-2-lambda⁵,4lambda⁵-catenadi(phosphazene) (P₂-Et) and 2-tert-butylimino-2-diethylamino-1,3-dimethyl-perhydro-1,3,2-diazaphosphorine (BEMP), optionally in the presence of a diluent, preferably an inert solvent, e.g. a hydrocarbon, an ether, such as tetrahydrofuran or 1,2-dimethoxyethane, a polar aprotic solvent, such as N,N-dimethylformamide, or a halogenated hydrocarbon, such as dichloromethane, or mixtures thereof, and optionally in the presence of a complexing agent, such as 1,3-dimethyl-3,4,5,6-tetrahydro-2(1H)-pyrimidinone (DMPU), hexamethylphosphoramide (HMPA) or tetramethylethylenediamine (TMEDA), in a temperature range of from −120° C. to 100° C., preferably from −80° C. to 50° C. Such processes are known in the literature and are described, for example, in J. Med. Chem., 2003 (46) 3021-3032; J. Org. Chem., 2003 (68) 1443-1446; J. Org. Chem., 2002 (67) 5216-5225 and J. Org. Chem., 2002 (67) 3065-3071.

2) The compounds of formula I wherein R¹, R², R³, R⁴, R⁵, R⁶ and R⁷ are as defined above, m is 1 or 2, and n is 1, can be prepared by processes known per se, by reacting e.g. a compound of formula Ic wherein R¹, R², R⁴, R⁵, R⁶ and R⁷ are as defined above, and m is 1 or 2,

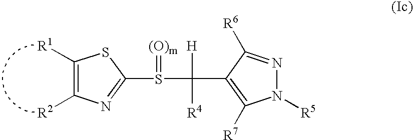

with a compound of formula R³—X, wherein R³ is as defined above and X is a suitable leaving group as defined in 1), in the presence of a base as defined in 1), optionally in the presence of a diluent as defined in 1), preferably an inert solvent, and optionally in the presence of a complexing agent as defined in 1), in a temperature range of from −120° C. to 100° C., preferably from −80° C. to 50° C.

3) The compounds of formula I wherein R¹, R², R³, R⁴, R⁵, R⁶ and R⁷ are as defined above, m is 1 or 2, and n is 1, can be prepared by processes known per se, by reacting e.g. a compound of formula Id wherein R¹, R², R³, R⁵, R⁶ and R⁷ are as defined above, and m is 1 or 2,

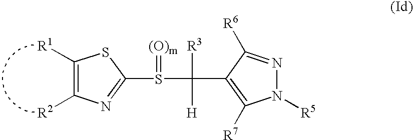

with a compound of formula R⁴—X, wherein R⁴ is as defined above and X is a suitable leaving group as defined in 1), in the presence of a base as defined in 1), optionally in the presence of a diluent as defined in 1), preferably an inert solvent, and optionally in the presence of a complexing agent as defined in 1), in a temperature range of from −120° C. to 100° C., preferably from −80° C. to 50° C.

4) The compounds of formula I wherein R¹, R², R³, R⁴, R⁵, R⁶ and R⁷ are as defined above, m is 1 or 2, and n is 1, can, furthermore, be prepared by processes known per se, by reacting e.g. a compound of formula Ie wherein R¹, R², R³, R⁴, R⁵, R⁶ and R⁷ are as defined above,

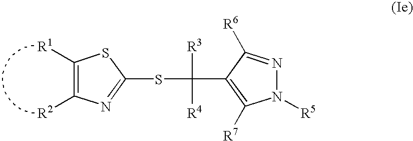

with a suitable organic or inorganic oxidising agent, e.g. a monopersulfate compound (Oxone®), a peroxy acid, such as 3-chloroperoxybenzoic acid, peracetic acid or hydrogen peroxide, an alkoxyperoxide or a periodate, such as sodium periodate, optionally in the presence of a diluent, such as a halogenated hydrocarbon, e.g. dichloromethane or 1,2-dichloroethane, an alcohol, e.g. methanol, a polar aprotic solvent, e.g. N,N-dimethylformamide, or a polar protic solvent, e.g. water or acetic acid, or a mixture thereof. The reactions are usually carried out in a temperature range of from −80° C. to 150° C., preferably from −20° C. to 120° C. Such processes are known in the literature and are described e.g. in J. Org. Chem., 2003 (68) 3849-3859; J. Med. Chem., 2003 (46) 3021-3032; J. Org. Chem., 2003 (68) 500-511; Bioorg. Med. Chem., 1999 (9) 1837-1844. One equivalent of oxidizing agent is required to convert a sulfide to the corresponding sulfoxide. Two equivalents of oxidizing agent are required to convert a sulfide to the corresponding sulfone. Furthermore, one equivalent of oxidizing agent is required to convert a sulfoxide to the corresponding sulfone.

5) The compounds of formula Ig wherein R¹, R², R³, R⁵, R⁶ and R⁷ are as defined above, can be prepared by reacting e.g. a compound of formula If wherein R¹, R², R⁵, R⁶ and R⁷ are as defined above,

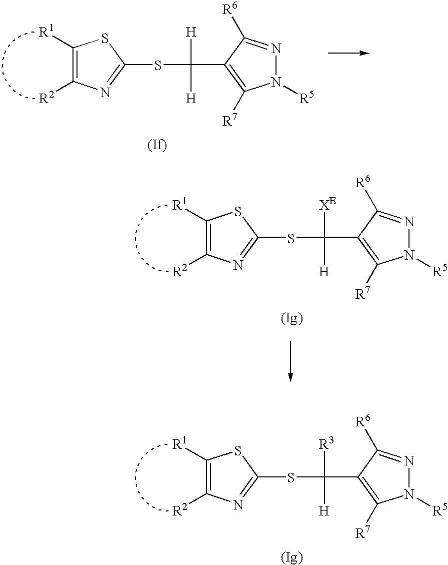

with a halogenating agent, e.g. bromine or an N-halosuccinimide, such as N-chlorosuccinimide or N-bromosuccinimide, to form a compound of formula Ig wherein R¹, R², R⁵, R⁶ and R⁷ are as defined above and X^{E} is halogen, optionally in the presence of a diluent, e.g. acetic acid or a halogenated hydrocarbon, such as CCl₄ or dichloromethane, in a temperature range of from −80° C. to 120° C., preferably from −20° C. to 60° C. The compound of formula Ig wherein R¹, R², R⁵, R⁶ and R⁷ are as defined above and X^{E} is halogen can then be oxidized directly as described in 4), or optionally in a second step be reacted with a compound of formula

M-R³

wherein R³ is as defined above, and M-R³ is a suitable salt or an organometal compound in which M is e.g. Li, MgBr, Na, K, Ag or tetraalkylammonium, optionally in the presence of a Lewis acid, e.g. SnCl₄, optionally in the presence of a complexing agent, e.g. hexamethylphosphoramide (HMPA) or 1,3-dimethyl-3,4,5,6-tetrahydro-2(1H)-pyrimidinone (DMPU), and optionally in the presence of a diluent, e.g. acetonitrile, dichloromethane, diethyl ether or tetrahydrofuran, in a temperature range of from −120° C. to 100° C., preferably from −80° C. to 80° C. Such processes are known in the literature and are described, for example, in J. Chem. Soc. Perkin Trans., 1995 (22) 2845-2848; Liebigs Annalen, 1993, 49-54; J. Org. Chem., 1986 (51) 3447-3451 and Synlett., 2000, 658-662.

6) The compounds of formula Ie as defined in 4), can be prepared, for example, by reacting e.g. a compound of formula If as defined in 5)

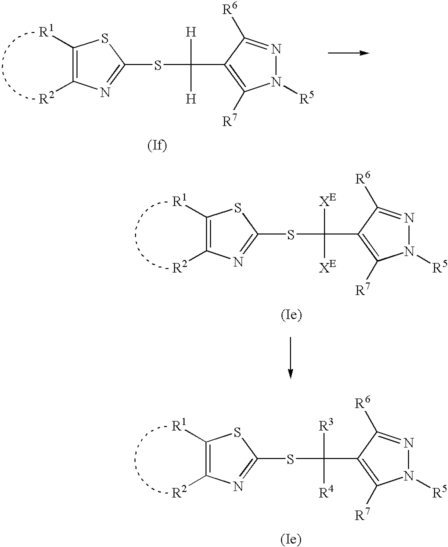

with a halogenating agent as defined in 5), to form a compound of formula Ie wherein R¹, R², R⁵, R⁶ and R⁷ are as defined above and X^{E} is halogen, optionally in the presence of a diluent as defined in 5), in a temperature range of from −80° C. to 120° C., preferably from −20° C. to 60° C. The compound of formula Ie wherein R¹, R², R⁵, R⁶ and R⁷ are as defined above and X^{E} is halogen can then be oxidized directly as described in 4), or optionally in a second or third step be reacted with compounds of formula

M-R³ and/or M-R⁴

wherein R³ and R⁴ are as defined above, and M-R³ and/or M-R⁴ are a suitable salt or an organometal compound in which M is e.g. Li, MgBr, Na, K, Ag or tetraalkylammonium, optionally in the presence of a Lewis acid, e.g. SnCl₄, optionally in the presence of a complexing agent, e.g. hexamethylphosphoramide (HMPA) or 1,3-dimethyl-3,4,5,6-tetrahydro-2(1H)-pyrimidinone (DMPU), and optionally in the presence of a diluent, e.g. acetonitrile, dichloromethane, diethyl ether or tetrahydrofuran, in a temperature range of from −120° C. to 100° C., preferably from −80° C. to 80° C. Such processes are known in the literature and are described, for example, in J. Chem. Soc. Perkin Trans., 1995 (22) 2845-2848; Liebigs Annalen, 1993, 49-54; J. Org. Chem., 1986 (51) 3447-3451 and Synlett., 2000, 658-662.

7) The compounds of formula Ie as defined in 4), can also be prepared from a compound of formula II wherein R¹ and R² are as defined above and X^{A} is a suitable leaving group such as halogen, e.g. bromide or chloride, or an alkyl-, aryl- or haloalkylsulfonate, e.g. methylsulfonate, p-toluenesulfonate or trifluoromethylsulfonate, by reaction with thiourea, optionally in the presence of a diluent e.g. a halogenated hydrocarbon, such as dichloromethane, an aromatic hydrocarbon, such as toluene, an alcohol, such as methanol or ethanol, a polar aprotic solvent, such as dimethylsulfoxide, N—N-dimethylfommamide or acetonitrile, an ether, such as tetrahydrofuran, or a mixture thereof, in a temperature range of from 0° C. to 180° C., preferably from 20° C. to 100° C., to give an isothiourea intermediate of formula III,

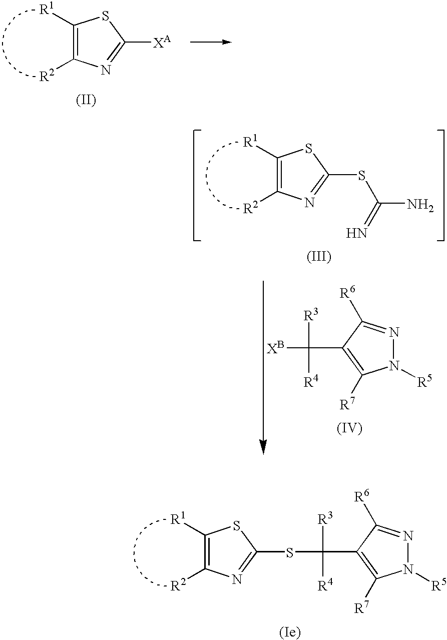

which is reacted with a compound of formula IV wherein R³, R⁴, R⁵, R⁶ and R⁷ are as defined above and X^{B} is a suitable leaving group such as halogen, e.g. bromide or chloride, or an alkyl-, aryl- or haloalkylsulfonate, e.g. methylsulfonate, p-toluenesulfonate or trifluoromethylsulfonate, in the presence of a base e.g. a metal hydride, preferably an alkali metal hydride, such as sodium hydride, a metal alkoxide, such as potassium tert-butoxide, an alkali metal hydroxide, such as sodium hydroxide, an alkali metal carbonate, such as potassium carbonate, or an organic base, such as triethylamine, pyridine or 1,8-diazabicyclo[5.4.0]-7-undecene (DBU), optionally in the presence of a diluent e.g. a halogenated hydrocarbon, such as dichloromethane, an aromatic hydrocarbon, such as toluene, an alcohol, such as methanol or ethanol, a polar aprotic solvent, such as dimethylsulfoxide, N—N-dimethylformamide or acetonitrile, an ether, such as tetrahydrofuran, or a mixture thereof, in a temperature range of from 0° C. to 180° C., preferably from 20° C. to 100° C. Such processes are known in the literature and are described, for example, in WO 04/013106.

8) The compounds of formula Ie as defined in 4) can also be prepared by reacting a compound of formula IV as defined in 7), with thiourea, optionally in the presence of a diluent e.g. an alcohol, such as ethanol, or a polar aprotic solvent, such as acetonitrile, optionally in the presence of an alkali iodide, e.g. sodium iodide or potassium iodide, in a temperature range of from −30° C. to 100° C., preferably from 0° C. to 80° C., to give an isothiourea intermediate of formula VI,

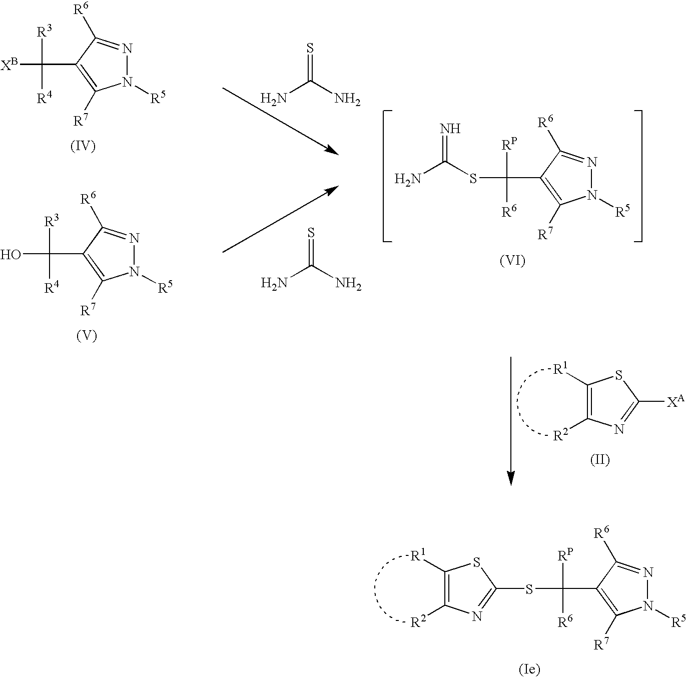

which is reacted with a compound of formula II as defined in 7), in the presence of a base, such as a carbonate, e.g. potassium carbonate, sodium carbonate or potassium bicarbonate, or a hydroxide, e.g. potassium hydroxide, or an alkoxide, e.g. sodium alkoxide, optionally in the presence of a diluent, such as an alcohol, e.g. ethanol, an ether, e.g. 1,4-dioxane or tetrahydrofuran, a polar aprotic solvent, such as acetonitrile or N,N-dimethylformamide, a protic solvent, such as water, or a mixture of thereof, e.g. a mixture of 1,4-dioxane and water, in a temperature range of from 20° C. to 200° C., preferably from 50° C. to 150° C., optionally in the presence of an inert gas e.g. nitrogen, and optionally under microwave irradiation. Such processes are known in the literature and are described, for example, in WO 04/013106.

9) A further method of preparing intermediates of formula VI as defined in 8) is to react a compound of formula V wherein R³, R⁴, R⁵, R⁶ and R⁷ are as defined above, with thiourea in the presence of an acid, for example a mineral acid, such as hydrochloric acid or hydrobromic acid, or sulfuric acid, or an organic acid, such as trifluoroacetic acid, and optionally in the presence of a diluent, such as an ether, e.g. 1,4-dioxane or tetrahydrofuran, a polar aprotic solvent, such as acetonitrile or N,N-dimethylformamide, a protic solvent, such as water, or a mixture of thereof, e.g. a mixture of 1,4-dioxane and water, in a temperature range of from 20° C. to 270° C., preferably from 20° C. to 150° C., optionally under microwave irradiation. Such processes are known in the literature and are described, for example, in Buchwald and Neilsen, JACS, 110(10), 3171-3175 (1988); Frank and Smith, JACS, 68, 2103-2104 (1946); Vetter, Syn. Comm., 28, 3219-3233 (1998). The intermediate VI is then reacted with a compound of formula II as defined in 7) to yield a compound of formula Ie as described in 8).

10) The compounds of formula Ie as defined in 4), can also be prepared from a compound of formula VII wherein R¹ and R² are as defined above

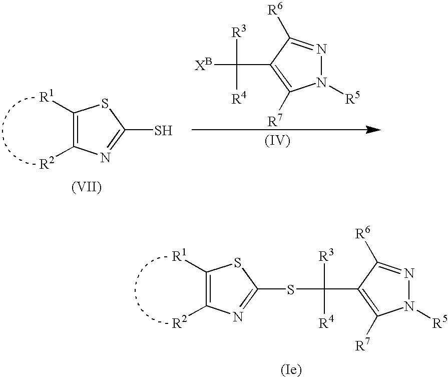

by reaction with a compound of formula IV as defined in 7), in the presence of a base, e.g. a carbonate, such as potassium carbonate, an alkoxide, such as sodium methoxide, a hydroxide, such as sodium hydroxide, optionally in the presence of a diluent, e.g. a polar aprotic solvent, such as N,N-dimethylformamide, acetonitrile or dimethylsulfoxide, an alcohol, such as methanol, or a protic solvent, such as water, in a temperature range of from 0° C. to 120° C., preferably from 20° C. to 100° C., and optionally under an inert atmosphere, e.g. nitrogen. Similar processes are known in the literature and are described, for example in J. Med. Chem. 2002 (45) 4282-4299, J. Med. Chem. 2002 (45) 3905-3927, Archiv der Pharmazie 2004 (337) 549-555.

11) The compounds of formula Ie as defined in 4) can also be prepared from a compound of formula II as defined in 7)

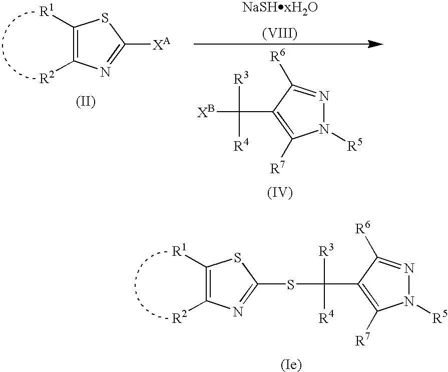

by reaction with a sodium hydrosulfide of formula VIII optionally in the presence of a base and optionally in the presence of a diluent, e.g. a halogenated hydrocarbon, such as dichloromethane, an alcohol, such as ethanol, a polar aprotic solvent, such as N—N-dimethylformamide, an ether, such as tetrahydrofuran, or a mixture thereof, followed by reaction with a compound of formula IV as defined in 7), in a temperature range of from −20° C. to 120° C., preferably from 0° C. to 80° C. To the reaction may optionally be added a radical-generating agent e.g. Rongalit (CH₂(OH)SO₂Na.2H₂O). The base can be, for example, an alkyl-lithium compound, such as methyl-lithium, n-butyl-lithium and tert-butyl-lithium, a lithium dialkylamide, such as lithium diisopropylamide, a metal hydride, preferably an alkali metal hydride, such as sodium hydride, or an alkali metal amide, such as sodium amide, a metal bis(tri(C₁-C₆alkyl)silyl)amide, such as lithium bis(trimethylsilyl)amide, a metal alkoxide, such as potassium tert-butoxide, an alkali metal carbonate such as potassium carbonate, an organic base such as triethylamine, pyridine or 1,8-diazabicyclo[5.4.0]-7-undecene (DBU). Similar processes are known in the literature and are described, for example in US 2004/0110749.

12) The compounds of formula Ie as defined in 4), can also be prepared from a compound of formula II as defined in 7)

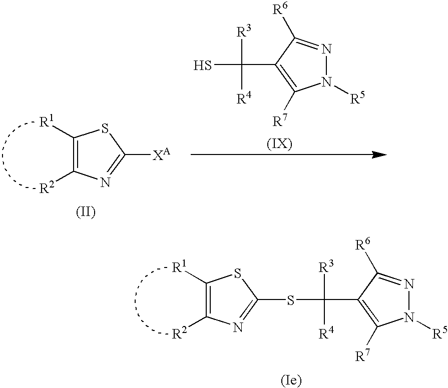

by reacting with a compound of formula IX wherein R³, R⁴, R⁵, R⁶ and R⁷ are as defined above, in the presence of a base, such as a metal hydride, preferably an alkali metal hydride, such as sodium hydride, a lithium dialkylamide, such as lithium diisopropylamide, an alkali metal amide, such as sodium amide, a metal bis(tri(C₁-C₆alkyl)silyl)amide, such as lithium bis(trimethylsilyl)amide, a metal alkoxide, such as potassium tert-butoxide, an alkali metal carbonate such as potassium carbonate, or an organic base such as triethylamine, pyridine or 1,8-diazabicyclo[5.4.0]-7-undecene (DBU), optionally in the presence of a diluent e.g. a halogenated hydrocarbon, such as dichloromethane, an alcohol, such as ethanol, a polar aprotic solvent, such as N—N-dimethylformamide, an ether, such as tetrahydrofuran, or a mixture thereof, in a temperature range of from 0° C. to 120° C., preferably from 20° C. to 80° C. Similar processes are known in the literature and described e.g. in Angew. Chem. Inter. Ed. Engl., 2003 (42) 3515-3520.

13) The compounds of formula Ih wherein R¹, R², R³, R⁴, R⁵, R⁶ and R⁷ are as defined above, m is 1 or 2, and n is 1, can also be prepared from a compound of formula IV wherein R³, R⁴, R⁵, R⁶ and R⁷ are defined as above and X^{B} is halogen, such as bromide,

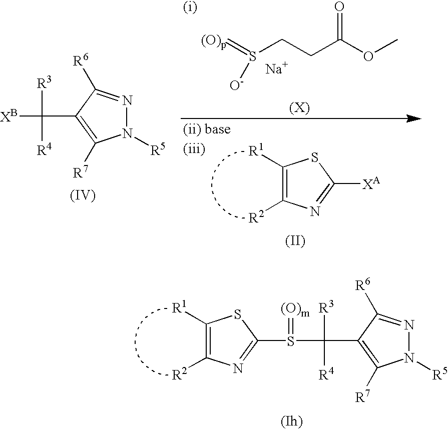

by reacting sequentially with a compound of formula X wherein p is 0 or 1 in the presence of a diluent e.g. a halogenated hydrocarbon, such as dichloromethane, an aromatic hydrocarbon, such as toluene, an alcohol, such as methanol or ethanol, a polar aprotic solvent, such as dimethylsulfoxide, N—N-dimethylformamide or acetonitrile, an ether, such as tetrahydrofuran, or a mixture thereof, in the presence of a base, e.g. a metal alkoxide, such as sodium methoxide or potassium tert-butoxide, an alkali metal hydroxide, such as sodium hydroxide, an alkali metal carbonate, such as potassium carbonate, an alkali metal disilazane, such as sodium hexamethyldisilazane (NaHMDS), or an organic base, such as triethylamine, pyridine or 1,8-diazabicyclo[5.4.0]-7-undecene (DBU), and then with a compound of formula II as defined in 7), in a temperature range of from −80° C. to 120° C., preferably from −80° C. to 80° C. Analogous processes are known in the literature and are described, for example, in Tetrahedron Lett., 2002 (43) 8479-8483.

14) The compounds of formula Ih wherein R¹, R², R³, R⁴, R⁵, R⁶ and R⁷ are as defined above, and m is 1 or 2, can also be prepared from a compound of formula II as defined in 7)

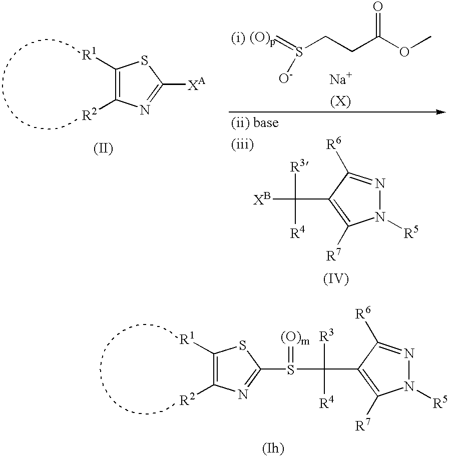

by reacting sequentially with a compound of formula X wherein p is 0 or 1 in the presence of a diluent as defined in 13), in the presence of a base as defined in 13), and then with a compound of formula IV as defined in 13).

15) Alternatively, the compounds of formula Ih wherein R¹, R², R³, R⁴, R⁵, R⁶ and R⁷ are as defined above, and m is 1 or 2, can also be prepared from a compound of formula II as defined in 7)

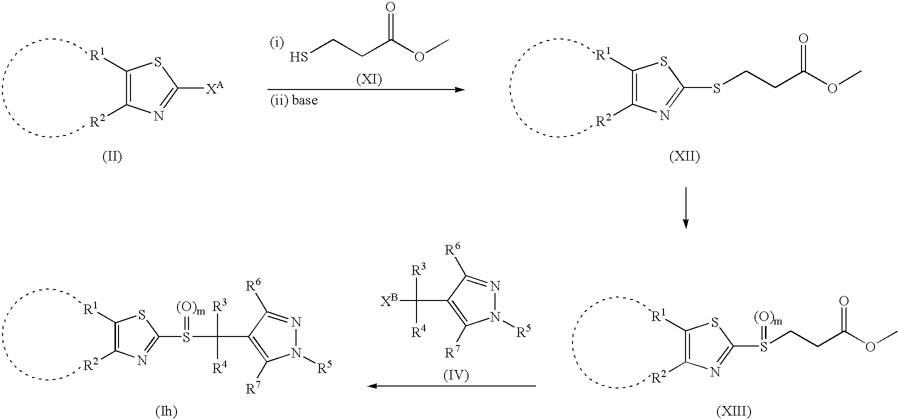

by reacting sequentially with a compound of formula XI in the presence of a diluent as defined in 13) and in the presence of a base as defined in 13). The intermediate XII is oxidised as described in 4) and the intermediate XIII wherein m is 1 or 2 is reacted with a compound of formula IV as defined in 13) in the presence of a diluent as defined in 13) and in the presence of a base as defined in 13). Analogous processes are known in the literature and are described, for example, in J. Org. Chem., 2005 (70) 2812-2815.

16) The compounds of formula IVa wherein X^{B} is halogen, such as bromide or chloride, can be prepared from a compound of formula XIV wherein R⁵, R⁶ and R⁷ are as defined above,

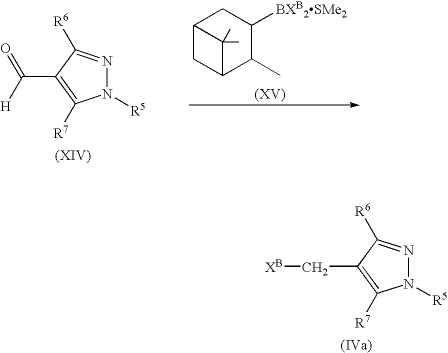

by reacting with reagent of formula XV wherein X^{B} is halogen, such as bromide or chloride, in the presence of a diluent e.g. a halogenated hydrocarbon, such as dichloromethane, a hydrocarbon, such as hexane, an alcohol, such as ethanol, a polar aprotic solvent, such as N—,N-dimethylformamide, an ether, such as tetrahydrofuran, or a mixture thereof, in a temperature range of from −20° C. to 120° C., preferably from 0° C. to 80° C. The preparation of aromatic benzyl halides using a similar procedure is described in Tetrahedron Lett. 2000 (41) 5161-5164. The preparation of aldehydes of formula XI is described in WO 04/013106; and the preparation of the reagent of formula XII is described in J. Org. Chem. 1980 (45) 384-389.

The compounds of formula II are commercially available or can be prepared according to methods known in the literature e.g. J. Amer. Chem. Soc. 1953 (75) 102-4; J. Het. Chem. 1978 (15) 1361-6; Comprehensive Heterocyclic Chemistry II, 1996, volume 3, 373-474.

The compounds of formula IV are commercially available or can be prepared according to methods known in the literature e.g. WO 04/014138.

The compounds of formula VII are commercially available or can be prepared according to methods known in the literature e.g. G. Vernin in Heterocyclic Compounds ed. J. V. Metzinger, Wiley, 1979, vol. 34, 260-271.

The compounds of formula I according to the invention can be used as herbicides in unmodified form, as made, but they are generally formulated into herbicidal compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO Specifications for Plant Protection Products, 5th Edition, 1999. Such formulations can either be used directly or they are diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof. The active ingredients can also be contained in very fine microcapsules consisting of a polymer. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art in this connection. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octa-decanoic acid, octylamine acetate, oleic acid, oleylamine, o-xylene, phenol, polyethylene glycol (PEG400), propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like. Water is generally the carrier of choice for diluting the concentrates. Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances, as described, for example, in CFR 180.1001. (c) & (d).

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in “McCutcheon's Detergents and Emulsifiers Annual” MC Publishing Corp., Ridgewood N.J., 1981.

Further adjuvants that can usually be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and also liquid and solid fertilisers.

The compositions according to the invention can additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhône-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being of importance. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000.

The application and action of the oil additives can be further improved by combination with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltriloxanes which are commercially available e.g. as Silwet L-77®, and also perfluorinated surfactants. The concentration of the surface-active substances in relation to the total additive is generally from 1 to 30% by weight. Examples of oil additives consisting of mixtures of oil or mineral oils or derivatives thereof with surfactants are Edenor ME SU®, Turbocharge® (Syngenta AG, CH) or ActipronC (BP Oil UK Limited, GB).

If desired, it is also possible for the mentioned surface-active substances to be used in the formulations on their own, that is to say without oil additives.

Furthermore, the addition of an organic solvent to the oil additive/surfactant mixture may contribute to an additional enhancement of action. Suitable solvents are, for example, Solvesso® (ESSO) or Aromatic Solvent® (Exxon Corporation). The concentration of such solvents can be from 10 to 80% by weight of the total weight. Oil additives that are present in admixture with solvents are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada).

In addition to the oil additives listed above, for the purpose of enhancing the action of the compositions according to the invention it is also possible for formulations of alkylpyrrolidones (e.g. Agrimax®) to be added to the spray mixture. Formulations of synthetic latices, e.g. polyacrylamide, polyvinyl compounds or poly-1-p-menthene (e.g. Bond®, Courier® or Emerald®) may also be used. It is also possible for solutions that contain propionic acid, for example Eurogkem Pen-e-trate®, to be added to the spray mixture as action-enhancing agent.

The herbicidal compositions generally comprise from 0.1 to 99% by weight, especially from 0.1 to 95% by weight, compounds of formula I and from 1 to 99.9% by weight of a formulation adjuvant which preferably includes from 0 to 25% by weight of a surface-active substance. Whereas commercial products will preferably be formulated as concentrates, the end user will normally employ dilute formulations.

The rates of application of compounds of formula I may vary within wide limits and depend on the nature of the soil, the method of application (pre- or post-emergence; seed dressing; application to the seed furrow; no tillage application etc.), the crop plant, the grass or weed to be controlled, the prevailing climatic conditions, and other factors governed by the method of application, the time of application and the target crop. The compounds of formula I according to the invention are generally applied at a rate of from 10 to 2000 g/ha, especially from 50 to 1000 g/ha.

Preferred formulations have especially the following compositions (%=percent by weight):

Emulsifiable concentrates:

| | |
|---|---|
| active ingredient: | 1 to 95%, preferably 60 to 90% |
| surface-active agent: | 1 to 30%, preferably 5 to 20% |
| liquid carrier: | 1 to 80%, preferably 1 to 35% |

Dusts:

| | |
|---|---|
| active ingredient: | 0.1 to 10%, preferably 0.1 to 5% |
| solid carrier: | 99.9 to 90%, preferably 99.9 to 99% |

Suspension concentrates:

| | |
|---|---|
| active ingredient: | 5 to 75%, preferably 10 to 50% |
| water: | 94 to 24%, preferably 88 to 30% |
| surface-active agent: | 1 to 40%, preferably 2 to 30% |

Wettable powders:

| | |
|---|---|
| active ingredient: | 0.5 to 90%, preferably 1 to 80% |
| surface-active agent: | 0.5 to 20%, preferably 1 to 15% |
| solid carrier: | 5 to 95%, preferably 15 to 90% |

Granules:

| | |
|---|---|
| active ingredient: | 0.1 to 30%, preferably 0.1 to 15% |
| solid carrier: | 99.5 to 70%, preferably 97 to 85% |

The following Examples further illustrate, but do not limit, the invention.

### Formulation Examples for Herbicides of Formula I (%=% by Weight)

| F1. Emulsifiable concentrates | a) | b) | c) | d) |
|---|---|---|---|---|
| active ingredient | 5% | 10% | 25% | 50% |
| calcium dodecylbenzenesulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP | — | — | 10% | 20% |
| arom. hydrocarbon mixture | 85% | 78% | 55% | 16% |
| C₉-C₁₂ | | | | |

Emulsions of any desired concentration can be obtained from such concentrates by dilution with water.

| F2. Solutions | a) | b) | c) | d) |
|---|---|---|---|---|
| active ingredient | 5% | 10% | 50% | 90% |
| 1−methoxy−3−(3−methoxy− | — | 20% | 20% | — |
| propoxy)-propane | | | | |
| polyethylene glycol MW 400 | 20% | 10% | — | — |
| NMP | — | — | 30% | 10% |
| arom. hydrocarbon mixture | 75% | 60% | — | — |
| C₉-C₁₂ | | | | |

The solutions are suitable for use in the form of microdrops.

| F3. Wettable powders | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

| F4. Coated granules | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride and applied to the carrier by spraying, and the solvent is then evaporated off in vacuo.

| F5. Coated granules | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is uniformly applied, in a mixer, to the carrier moistened with polyethylene glycol. Non-dusty coated granules are obtained in this manner.

| F6. Extruder granules | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants, and the mixture is moistened with water. The mixture is extruded and then dried in a stream of air.

| F7. Dusts | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

| F8. Suspension concentrates | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, giving a suspension concentrate from which suspensions of any desired concentration can be obtained by dilution with water.

The invention relates also to a method for the selective control of grasses and weeds in crops of useful plants, wherein the useful plants or the area of cultivation or locus thereof is treated with the compounds of formula I.

Useful plant crops in which the composition according to the invention can be used include especially maize, soybeans, cotton, cereals, e.g. wheat and barley, rice, sugar cane, sugar beet, sunflowers and rape. Crops are to be understood as also including those crops which have been rendered tolerant to herbicides or classes of herbicides (e.g. ALS-, GS-, EPSPS-, PPO- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The weeds to be controlled may be both monocotyledonous and dicotyledonous weeds, for example Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus, Abutilon, Sida, Xanthium, Amaranthus, Chenopodium, Ipomoea, Chrysanthemum, Galium, Viola and Veronica.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgardo (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidally effective Cry3 protein while at the same time being tolerant to glyphosate. Crops are also to be understood as being those which are obtained by conventional methods of breeding or genetic engineering and contain so-called output traits (e.g. improved storage stability, higher nutritional value and improved flavour).

Areas under cultivation include land on which the crop plants are already growing and land intended for cultivation with those crop plants.

The compounds of formula I according to the invention can also be used in combination with other herbicides. In particular, the following mixtures of the compound of formula I are important:

Mixtures of a compound of the formula I with S-metolachlor (549).

Mixtures of a compound of the formula I with a triazine (e.g. compound of formula I+ametryn (20), compound of formula I+atrazine (37), compound of formula I+cyanazine (183), compound of formula I+dimethametryn (259), compound of formula I+metribuzin (554), compound of formula I+prometon (665), compound of formula I+prometryn (666), compound of formula I+propazine (672), compound of formula I+simazine (730), compound of formula I+simetryn (732), compound of formula I+terbumeton (774), compound of formula I+terbuthylazine (775), compound of formula I+terbutryn (776), compound of formula I+trietazine (831)). Particularly preferred are mixtures of a compound of formula I with atrazine, metribuzin, prometryn or with terbuthylazine.

Mixtures of a compound of formula I with an HPPD inhibitor (e.g. compound of formula I+tembotrione (CAS RN 335104-84-2), compound of formula I+topramezone (CAS RN 210631-68-8), compound of formula I+4-hydroxy-3-[[2-[(2-methoxyethoxy)-methyl]-6-(trifluoromethyl)-3-pyridinyl]carbonyl]-bicyclo[3.2.1]oct-3-en-2-one (CAS RN 352010-68-5), compound of formula I+4-hydroxy-3-[[2-(3-methoxypropyl)-6-(difluoromethyl)-3-pyridinyl]carbonyl]-bicyclo[3.2.1]oct-3-en-2-one, compound of formula I+isoxaflutole (479), compound of formula I+mesotrione (515), compound of formula I+sulcotrione (747)).

Mixtures of a compound of the formula I with an HPPD inhibitor and a triazine.

Mixtures of a compound of formula I with glyphosate (419).

Mixtures of a compound of formula I with glyphosate (419) and an HPPD inhibitor (e.g. compound of formula I+glyphosate+tembotrione (CAS RN 335104-84-2), compound of formula I+glyphosate+topramezone (CAS RN 210631-68-8), compound formula I+glyphosate+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), compound of formula I+glyphosate+4-hydroxy-3-[[2-(3-methoxypropyl)-6-(difluoromethyl)-3-pyridinyl]carbonyl]-bicyclo[3.2.1]oct-3-en-2-one, compound of formula I+glyphosate+isoxaflutole, compound of formula I+glyphosate+mesotrione, compound of formula I+glyphosate+sulcotrione).

Mixtures of a compound of formula I with glufosinate-ammonium (418).

Mixtures of a compound of formula I with glufosinate-ammonium (418) and an HPPD inhibitor (e.g. compound of formula I+glufosinate-ammonium+tembotrione (CAS RN 335104-84-2), compound of formula I+glufosinate-ammonium+topramezone (CAS RN 210631-68-8), compound of formula I+glufosinate-ammonium+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), compound of formula I+glufosinate-ammonium+4-hydroxy-3-[[2-(3-methoxypropyl)-6-(difluoromethyl)-3-pyridinyl]carbonyl]-bicyclo[3.2.1]oct-3-en-2-one, compound of formula I+glufosinate-ammonium+isoxaflutole, compound of formula I+glufosinate-ammonium+mesotrione, compound of formula I+glufosinate-ammonium+sulcotrione).

Mixtures of a compound of formula I with a triazolinone (e.g. compound of formula I+amicarbazone (21)).

Mixtures of a compound of formula I with an ALS inhibitor (e.g. compound of formula I+chlorsulfuron (147), compound of formula I+cinosulfuron (154), compound of formula I+cloransulam-methyl (164), compound of formula I+ethametsulfuron-methyl (306), compound of formula I+flazasulfuron (356), compound of formula I+foramsulfuron (402), compound of formula I+flumetsulam (374), compound of formula I+imazamethabenz-methyl (450), compound of formula I+imazamox (451), compound of formula I+imazapic (452), compound of formula I+imazapyr (453), compound of formula I+imazethapyr (455), compound of formula I+iodosulfuron-methyl-sodium (466), compound of formula I+metsulfuron-methyl (555), compound of formula I+nicosulfuron (577), compound of formula I+oxasulfuron (603), compound of formula I+primisulfuron-methyl (657), compound of formula I+prosulfuron (684), compound of formula I+pyrithiobac-sodium (709), compound of formula I+rimsulfuron (721), compound of formula I+sulfosulfuron (752), compound of formula I+thifensulfuron-methyl (795), compound of formula I+triasulfuron (817), compound of formula I+tribenuron-methyl (822), compound of formula I+trifloxysulfuron-sodium (833), compound of formula I+4-[(4,5-dihydro-3-methoxy-4-methyl-5-oxo)-1H-1,2,4-triazol-1-ylcarbonylsulfamoyl]-5-methylthiophene-3-carboxylic acid (BAY636)). Particularly preferred are mixtures of a compound of formula I with flazasulfuron, foramsulfuron, flumetsulam, imazapyr, imazethapyr, iodosulfuron-methyl-sodium, nicosulfuron, rimsulfuron, trifloxysulfuron-sodium or with 4-[(4,5-dihydro-3-methoxy-4-methyl-5-oxo)-1H-1,2,4-triazol-1-ylcarbonylsulfamoyl]-5-methylthiophene-3-carboxylic acid (BAY636).

Mixtures of a compound of formula I with a PPO inhibitor (e.g. compound of formula I+fomesafen (401), compound of formula I+flumioxazin (376), compound of formula I+sulfentrazone (749), compound of formula I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester) (CAS RN 353292-31-6). Particularly preferred are mixtures of a compound of formula I with flumioxazin, sulfentrazone or [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6).

Mixtures of a compound of formula I with paraquat dichloride (614).

Mixtures of a compound of formula I with pendimethalin (621) or a compound of formula I with trifluralin (836). Particularly preferred are mixtures of a compound of formula I with pendimethalin.

Mixtures of a compound of formula I with metamitron (521).

Mixtures of a compound of formula I with clomazone (159).

Mixtures of a compound of formula I with metazachlor (524).

Mixtures of a compound of formula I with clodinafop-propargyl (156) or a compound of formula I with pinoxaden.

The mixing partners of the compound of formula I may also be in the form of esters or salts, as mentioned e.g. in The Pesticide Manual, 13^{th} Edition (BCPC), 2003. The reference to glufosinate-ammonium also applies to glufosinate, the reference to cloransulam-methyl also applies to cloransulam, and the reference to pyrithiobac-sodium also applies to pyrithiobac, etc.

The mixing ratio of the compound of formula I to the mixing partner is preferably from 1:100 to 1000:1.

The mixtures can advantageously be used in the above-mentioned formulations (in which case “active ingredient” relates to the respective mixture of compound of formula I with the mixing partner).

Furthermore, the compounds of formula I according to the invention can also be used in combination with other herbicides: compound of formula I+acetochlor (5), compound of formula I+acifluorfen-sodium (7), compound of formula I+aclonifen (8), compound of formula I+acrolein (10), compound of formula I+alachlor (14), compound of formula I+alloxydim (18), compound of formula I+allyl alcohol, compound of formula I+amidosulfuron (22), compound of formula I+aminopyralid, compound of formula I+amitrole (25), compound of formula I+ammonium sulfamate (26), compound of formula I+anilofos (31), compound of formula I+asulam (36), compound of formula I+atraton, compound of formula I+azimsulfuron (43), compound of formula I+BCPC, compound of formula I+beflubutamid (55), compound of formula I+benazolin (57), compound of formula I+benfluralin (59), compound of formula I+benfuresate (61), compound of formula I+bensulfuron-methyl (64), compound of formula I+bensulide (65), compound of formula I+bentazone (67), compound of formula I+benzfendizone, compound of formula I+benzobicyclon (69), compound of formula I+benzofenap (70), compound of formula I+bifenox (75), compound of formula I+bilanafos (77), compound of formula I+bispyribac-sodium (82), compound of formula I+borax (86), compound of formula I+bromacil (90), compound of formula I+bromobutide (93), compound of formula I+bromoxynil (95), compound of formula I+butachlor (100), compound of formula I+butafenacil (101), compound of formula I+butamifos (102), compound of formula I+butralin (105), compound of formula I+butroxydim (106), compound of formula I+butylate (108), compound of formula I+cacodylic acid, compound of formula I+calcium chlorate, compound of formula I+cafenstrole (110), compound of formula I+carbetamide (117), compound of formula I+carfentrazone-ethyl (121), compound of formula I+CDEA, compound of formula I+CEPC, compound of formula I+chlorflurenol-methyl (133), compound of formula I+chloridazon (134), compound of formula I+chlorimuron-ethyl (135), compound of formula I+chloroacetic acid (138), compound of formula I+chlorotoluron (143), compound of formula I+chlorpropham (144), compound of formula I+chlorthal-dimethyl (148), compound of formula I+cinidon-ethyl (152), compound of formula I+cinmethylin (153), compound of formula I+cisanilide, compound of formula I+clethodim (155), compound of formula I+clomeprop (160), compound of formula I+clopyralid (162), compound of formula I+CMA, compound of formula I+4-CPB, compound of formula I+CPMF, compound of formula I+4-CPP, compound of formula I+CPPC, compound of formula I+cresol, compound of formula I+cumyluron (180), compound of formula I+cyanamide (182), compound of formula I+cycloate (187), compound of formula I+cyclosulfamuron (189), compound of formula I+cycloxydim (190), compound of formula I+cyhalofop-butyl (195), compound of formula I+2,4-D (211), compound of formula I+3,4-DA, compound of formula I+daimuron (213), compound of formula I+dalapon (214), compound of formula I+dazomet (216), compound of formula I+2,4-DB (217), compound of formula I+3,4-DB, compound of formula I+2,4-DEB, compound of formula I+desmedipham (225), compound of formula I+dicamba (228), compound of formula I+dichlobenil (229), compound of formula I+ortho-dichlorobenzene, compound of formula I+para-dichlorobenzene, compound of formula I+dichlorprop (234), compound of formula I+dichlorprop-P (235), compound of formula I+diclofop-methyl (238), compound of formula I+diclosulam (241), compound of formula I+difenzoquat metilsulfate (248), compound of formula I+diflufenican (251), compound of formula I+diflufenzopyr (252), compound of formula I+dimefuron (256), compound of formula I+dimepiperate (257), compound of formula I+dimethachlor (258), compound of formula I+dimethenamid (260), compound of formula I+dimethenamid-P, compound of formula I+dimethipin (261), compound of formula I+dimethylarsinic acid (264), compound of formula I+dinitramine (268), compound of formula I+dinoterb (272), compound of formula I+diphenamid (274), compound of formula I+diquat dibromide (276), compound of formula I+dithiopyr (280), compound of formula I+diuron (281), compound of formula I+DNOC (282), compound of formula I+3,4-DP, compound of formula I+DSMA, compound of formula I+EBEP, compound of formula I+endothal (295), compound of formula I+EPTC (299), compound of formula I+esprocarb (303), compound of formula I+ethalfluralin (305), compound of formula I+ethofumesate (311), compound of formula I+ethoxyfen, compound of formula I+ethoxysulfuron (314), compound of formula I+etobenzanid (318), compound of formula I+fenoxaprop-P-ethyl (339), compound of formula I+fentrazamide (348), compound of formula I+ferrous sulfate (353), compound of formula I+flamprop-M (355), compound of formula I+florasulam (359), compound of formula I+fluazifop-butyl (361), compound of formula I+fluazifop-P-butyl (362), compound of formula I+flucarbazone-sodium (364), compound of formula I+flucetosulfuron, compound of formula I+fluchloralin (365), compound of formula I+flufenacet (369), compound of formula I+flufenpyr-ethyl (371), compound of formula I+flumiclorac-pentyl (375), compound of formula I+fluometuron (378), compound of formula I+fluoroglycofenethyl (380), compound of formula I+flupropanate (383), compound of formula I+flupyrsulfuron-methyl-sodium (384), compound of formula I+flurenol (387), compound of formula I+fluridone (388), compound of formula I+fluorochloridone (389), compound of formula I+fluoroxypyr (390), compound of formula I+flurtamone (392), compound of formula I+fluthiacet-methyl (395), compound of formula I+fosamine (406), compound of formula I+halosulfuron-methyl (426), compound of formula I+haloxyfop (427), compound of formula I+haloxyfop-P (428), compound of formula I+HC-252 (429), compound of formula I+hexazinone (440), compound of formula I+imazaquin (454), compound of formula I+imazosulfuron (456), compound of formula I+indanofan (462), compound of formula I+iodomethane, compound of formula I+ioxynil (467), compound of formula I+isoproturon (475), compound of formula I+isouron (476), compound of formula I+isoxaben (477), compound of formula I+isoxachlortole, compound of formula I+karbutilate (482), compound of formula I+lactofen (486), compound of formula I+lenacil (487), compound of formula I+linuron (489), compound of formula I+MAA, compound of formula I+MAMA, compound of formula I+MCPA (499), compound of formula I+MCPA-thioethyl (500), compound of formula I+MCPB (501), compound of formula I+mecoprop (503), compound of formula I+mecoprop-P (504), compound of formula I+mefenacet (505), compound of formula I+mefluidide (507), compound of formula I+mesosulfuron-methyl (514), compound of formula I+metam (519), compound of formula I+metamifop (520), compound of formula I+methabenzthiazuron (526), compound of formula I+methylarsonic acid (536), compound of formula I+methyldymron (539), compound of formula I+methyl isothiocyanate (543), compound of formula I+metobenzuron (547), compound of formula I+metolachlor (548), compound of formula I+metosulam (552), compound of formula I+metoxuron (553), compound of formula I+MK-616 (559), compound of formula I+molinate (560), compound of formula I+monolinuron (562), compound of formula I+MSMA, compound of formula I+naproanilide (571), compound of formula I+napropamide (572), compound of formula I+naptalam (573), compound of formula I+neburon (574), compound of formula I+nonanoic acid (583), compound of formula I+norflurazon (584), compound of formula I+oleic acid (fatty acids) (593), compound of formula I+orbencarb (595), compound of formula I+orthosulfamuron, compound of formula I+oryzalin (597), compound of formula I+oxadiargyl (599), compound of formula I+oxadiazon (600), compound of formula I+oxaziclomefone (604), compound of formula I+oxyfluorfen (610), compound of formula I+pebulate (617), compound of formula I+penoxsulam (622), compound of formula I+pentachlorophenol (623), compound of formula I+pentanochlor (624), compound of formula I+pentoxazone (625), compound of formula I+pethoxamid (627), compound of formula I+petrolium oils (628), compound of formula I+phenmedipham (629), compound of formula I+picloram (645), compound of formula I+picolinafen (646), compound of formula I+piperophos (650), compound of formula I+potassium arsenite, compound of formula I+potassium azide, compound of formula I+pretilachlor (656), compound of formula I+prodiamine (661), compound of formula I+profluazol, compound of formula I+profoxydim (663), compound of formula I+propachlor (667), compound of formula I+propaanil (669), compound of formula I+propaquizafop (670), compound of formula I+propham (674), compound of formula I+propisochlor (667), compound of formula I+propoxycarbazone-sodium (679), compound of formula I+propyzamide (681), compound of formula I+prosulfocarb (683), compound of formula I+pyraclonil, compound of formula I+pyraflufen-ethyl (691), compound of formula I+pyrazolynate (692), compound of formula I+pyrazosulfuron-ethyl (694), compound of formula I+pyrazoxyfen (695), compound of formula I+pyribenzoxim (697), compound of formula I+pyributicarb (698), compound of formula I+pyridafol, compound of formula I+pyridate (702), compound of formula I+pyriftalid (704), compound of formula I+pyriminobac-methyl (707), compound of formula I+pyrimisulfan, compound of formula I+quinclorac (712), compound of formula I+quinrnerac (713), compound of formula I+quinoclamine (714), compound of formula I+quizalofop (717), compound of formula I+quizalofop-P (718), compound of formula I+sethoxydim (726), compound of formula I+siduron (727), compound of formula I+SMA, compound of formula I+sodium arsenite, compound of formula I+sodium azide, compound of formula I+sodium chlorate (734), compound of formula I+sulfometuron-methyl (751), compound of formula I+sulfuric acid (755), compound of formula I+tar oils (758), compound of formula I+2,3,6-TBA (759), compound of formula I+TCA-sodium (760), compound of formula I+tebuthiuron (765), compound of formula I+tepraloxydim (771), compound of formula I+terbacil (772), compound of formula I+thenylchlor (789), compound of formula I+thiazopyr (793), compound of formula I+thiobencarb (797), compound of formula I+tiocarbazil (807), compound of formula I+tralkoxydim (811), compound of formula I+tri-allate (816), compound of formula I+triaziflam (819), compound of formula I+tricamba, compound of formula I+triclopyr (827), compound of formula I+triflusulfuron-methyl (837), compound of formula I+trihydroxytriazine and compound of formula I+tritosulfuron (843).

The mixing partners of the compound of formula I may also be in the form of esters or salts, as mentioned e.g. in The Pesticide Manual, 13^{th} Edition (BCPC), 2003. The reference to acifluorfen-sodium also applies to acifluorfen, and the reference to bensulfuron-methyl also applies to bensulfuron, etc.

The mixing ratio of the compound of formula I to the mixing partner is preferably from 1:100 to 1000:1.

The mixtures can advantageously be used in the above-mentioned formulations (in which case “active ingredient” relates to the respective mixture of compound of formula I with the mixing partner).

The compounds of formula I according to the invention can also be used in combination with one or more safeners. The safeners can be cloquintocet-mexyl (CAS RN 99607-70-2) or a lithium, sodium, potassium, calcium, magnesium, aluminium, iron, ammonium, quaternary ammonium, sulfonium or phosphonium salt thereof such as those disclosed in WO 02/34048, fenchlorazole (CAS RN 103112-36-3), fenchlorazole-ethyl (CAS RN 103112-35-2), mefenpyr (CAS RN 135591-00-3), mefenpyr-diethyl (CAS RN 135590-91-9), isoxadifen (CAS RN 209866-92-2), isoxadifen-ethyl (CAS RN 163520-33-0), furilazole (CAS RN 121776-33-8) and the corresponding R isomer (CAS RN 121776-57-6), benoxacor (CAS RN 98730-04-2), dichlormid (CAS RN 37764-25-3), MON4660 (CAS RN 71526-07-3), oxabetrinil (CAS RN 74782-23-3), cyometrinil (CAS RN 78370-21-5) and the corresponding (Z) isomer (CAS RN 63278-33-1), fenclorim (CAS RN 3740-92-9), cyprosulfamide (CAS RN 221667-31-8), N-isopropyl-4-(2-methoxy-benzoylsulfamoyl)-benzamide (CAS RN 221668-34-4), naphtbalic anhydride (CAS RN 81-84-5) and flurazole (CAS RN 72850-64-7). Particularly preferred are mixtures of a compound of formula I with benoxacor (i.e. compound of formula I+benoxacor).

Preferably the mixing ratio of compound of formula I to safener is from 100:1 to 1:10, especially from 20:1 to 1:1.

The mixtures can advantageously be used in the above-mentioned formulations (in which case “active ingredient” relates to the respective mixture of compound of formula I with the safener).

The following Examples further illustrate, but do not limit, the invention.

### PREPARATION EXAMPLES

### Example I1

### Preparation of 1-methyl-5-(2,2,2-trifluoro-ethoxy)-3-trifluoromethyl-1H-pyrazole-4-carbaldehyde

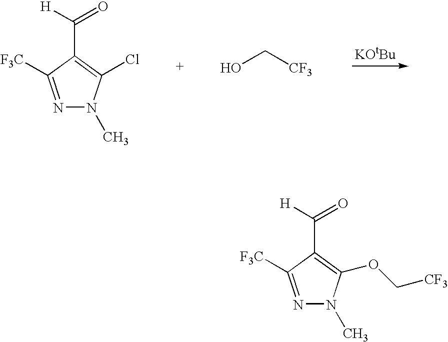

2,2,2-Trifluoroethanol (12.1 ml, 0.17 mol) was added dropwise to a solution of potassium tert-butoxide (1M in THF) (170 ml, 0.17 mol) in dry THF (80 ml) at 10° C. Then 5-chloro-1-methyl-3-trifluoromethyl-1H-pyrazole-4-carbaldehyde (30 g, 0.14 mol) (prepared according to WO 04/014138) in THF (40 ml) was added dropwise at 10-15° C. over 1 hour. At the end of the addition, the mixture was stirred at room temperature for one hour, then water (200 ml) and ethyl acetate (200 ml) were added. The phases were separated and the aqueous phase extracted 3 times with ethyl acetate. The combined organic extracts were washed with brine, dried over magnesium sulfate and concentrated to give the product (35.9 g, 92% yield).

¹H-NMR (400 MHz, CDCl₃): 3.8 (s, 3H, CH₃), 4.9-5.0 (q, 2H, CH₂), 9.85 (s, 1H, CHO).

### Example I2

### Preparation of [1-methyl-5-(2,2,2-trifluoro-ethoxy)-3-trifluoromethyl-1H-pyrazol-4-yl]-methanol

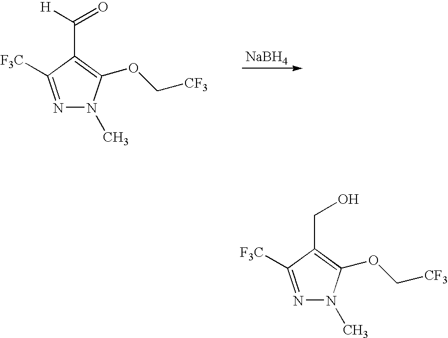

Sodium borohydride (2.95 g, 78 mmol) was added in portions to a solution of 1-methyl-5-(2,2,2-trifluoro-ethoxy)-3-trifluoromethyl-1H-pyrazole-4-carbaldehyde (21.5 g, 78 mmol) (see Example I1) in methanol (200 ml) at 0° C. The solution was stirred at 8-15° C. for 2 hours, then concentrated and the residue partitioned between dichloromethane and water. The organic phase was washed with sodium bicarbonate, brine, dried over magnesium sulfate and concentrated to give the product as a white solid (20.5 g, 94% yield).

¹H-NMR (400 MHz, CDCl₃): 3.8 (3H, s, CH₃), 4.5 (2H, s, CH₂), 4.75 (2H, q, CH₂).

### Example I3

### Preparation of 4-bromomethyl-1-methyl-5-(2,2,2-trifluoro-ethoxy-3-trifluoromethyl-1H-pyrazole

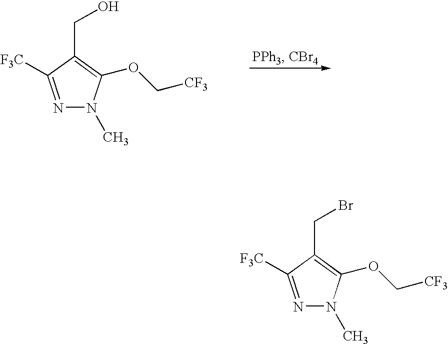

[1-Methyl-5-(2,2,2-trifluoro-ethoxy)-3-trifluoromethyl-1H-pyrazol-4-yl]-methanol (20.2 g, 73 mmol) (see Example I2) was dissolved in dichloromethane (200 ml) and cooled to 0° C. before triphenyl phosphine (20.9 g, 80 mmol) and carbon tetrabromide (23.2 g, 70 mmol) were added. The mixture was stirred for 2 hours and then concentrated. The residue was purified by chromatography on silica gel (eluent: 10% ethyl acetate in hexane) to give the desired product as a yellow oil (21.53 g, 87% yield) which solidified partially on refrigeration.

¹H-NMR (400 MHz, CDCl₃): 3.75 (s, 3H, CH₃), 4.40 (s, 2H, CH₂), 4.68 (q, 2H, CH₂).

The following compounds were also prepared according to the methods in Example I1, Example I2 and Example I3:

4-Bromomethyl-5-(3-fluoro-propoxy)-1-methyl-3-trifluoromethyl-1H-pyrazole was prepared using 3-fluoro-propan-1-ol as reagent in Example X1.

4-Bromomethyl-5-(2-fluoro-1-fluoromethyl-ethoxy)-1-methyl-3-trifluoromethyl-1H-pyrazole was prepared using 1,3-difluoro-propan-2-ol as reagent in Example X1.

4-Bromomethyl-1-methyl-5-(2,2,3,3-tetrafluoro-propoxy)-3-trifluoromethyl-1H-pyrazole was prepared using 2,2,3,3-tetrafluoro-propan-1-ol as reagent in Example X1.

4-Bromomethyl-5-(2-fluoro-1-methyl-ethoxy)-1-methyl-3-trifluoromethyl-1H-pyrazole was prepared using 1-fluoro-propan-2-ol as reagent in Example X1.

4-Bromomethyl-1-methyl-3-trifluoromethyl-5-(2,2,2-trifluoro-1-methyl-ethoxy)-1H-pyrazole was prepared using 1,1,1-trifluoro-propan-2-ol as reagent in Example X1.

### Example I4

### Alternative preparation of 4-bromomethyl-1-methyl-5-(2,2,2-trifluoro-ethoxy)-3-trifluoromethyl-1H-pyrazole

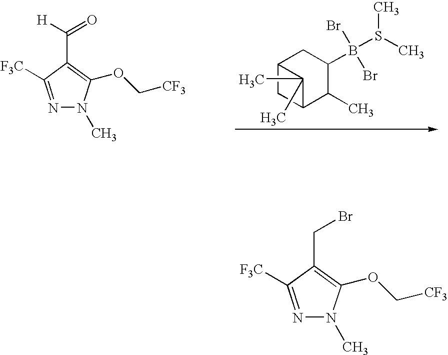

A solution of isopinocampheyl-boron dibromide dimethylsulfide complex (4.4 g, 12 mmol) (prepared according to J. Org. Chem. 1980 (45) 384-389) in dichloromethane (10 ml) was added over a period of 10 minutes to a solution of 1-methyl-5-(2,2,2-trifluoro-ethoxy)-3-trifluoromethyl-1H-pyrazole-4-carbaldehyde (3.0 g, 10.8 mmol) (see Example I1) in dry hexane (15 ml). The reaction mixture was stirred at room temperature for 3 hours. The solid was removed by filtration and washed with hexane/dichloro-methane (ratio 8:2, 2×10 ml). The organic phases were combined, diluted with diethyl ether (50 ml), washed twice with water, then with brine, dried over magnesium sulfate and concentrated. The residue was purified by chromatography on silica gel (eluent 0-50% ethyl acetate in hexane). The product was obtained as a colourless oil which solidified on standing (3.05 g, 83% yield).

The following compounds were also prepared according to this procedure:

4-Bromomethyl-5-chloro-1-methyl-3-trifluoromethyl-1H-pyrazole was prepared from 5-chloro-1-methyl-3-trifluoromethyl-1H-pyrazole-4-carbaldehyde (prepared according to WO 04/014138).

¹H-NMR (400 MHz, CDCl₃): 3.9 (s, 3H, CH₃), 4.4 (s, 2H, CH₂).

4-Bromomethyl-5-fluoro-1-methyl-3-trifluoromethyl-1H-pyrazole was prepared from 5-fluoro-1-methyl-3-trifluoromethyl-1H-pyrazole-4-carbaldehyde (prepared according to WO 04/014138).

¹H-NMR (400 MHz, CDCl₃): 3.8 (s, 3H, CH₃), 4.45 (s, 2H, CH₂).

### Example I5

### Preparation of 4-chloromethyl-1-methyl-5-(2,2,2-trifluoro-ethoxy)-3-trifluoromethyl-1H-pyrazole

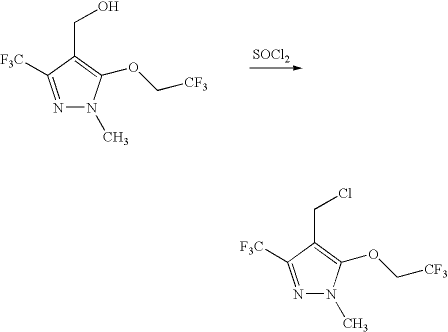

To a solution of [1-methyl-5-(2,2,2-trifluoro-ethoxy)-3-trifluoromethyl-1H-pyrazol-4-yl]-methanol (50 g, 0.14 mol) (see Example I2) in dichloromethane (300 ml), was slowly added thionyl chloride (17 ml, 0.17 mol) The mixture was stirred for 2 hours at room temperature before being concentrated. Twice, the residue was taken up in toluene and was concentrated again to remove excess of thionyl chloride. The crude product (53.5 g) was used without further purification.

### Example I6

### Preparation of 5-(2-fluoro-allyloxy)-1-methyl-3-trifluoromethyl-1H-pyrazole

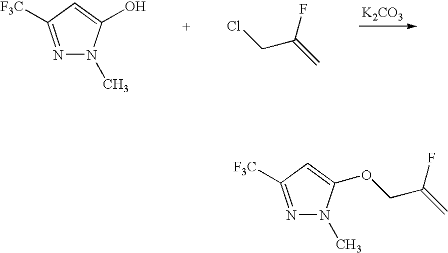

2-Methyl-5-trifluoromethyl-2H-pyrazol-3-ol (1.1 g, 6.6 mmol) and potassium carbonate (1.37 g, 9.9 mmol) were suspended in dry DMF (9 ml) and cooled to 0° C. 3-Chloro-2-fluoro-propene (0.69 g, 7.3 mmol) was added dropwise and the mixture was stirred for 16 hours. Water (35 ml) was added and the mixture was extracted 3 times with ethyl acetate. The combined organic extracts were washed with water and brine, dried over magnesium sulfate and concentrated. The residue was purified by chromatography on silica gel (eluent: hexane/ethyl acetate 4:1) to give the product as colourless liquid (1.13 g, 76% yield).

¹H-NMR (400 MHz, CDCl₃): 3.71 (s, 3H, CH₃), 4.59 (d, 2H, CH₂), 4.74 (dd, 1H, CH), 4.94 (dd, 1H, CH), 5.82 (s, 1H, CH).

### Example I7

### Preparation of 4-chloromethyl-5-(2-fluoro-allyloxy)-1-methyl-3-trifluoromethyl-1H-pyrazole

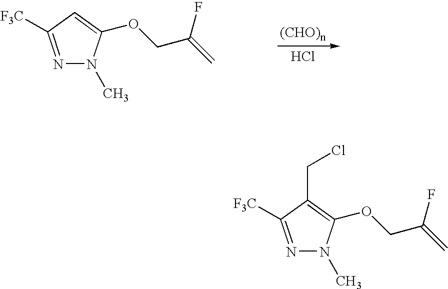

Parafomaldehyde (0.37 g, 4.1 mmol) was added to a solution of 5-(2-fluoro-allyloxy)-1-methyl-3-trifluoromethyl-1H-pyrazole (1.0 g, 4.5 mmol) (see Example I6) in glacial acetic acid (20 ml), followed by addition of concentrated hydrochloric acid (4 ml). The reaction was stirred at 80° C. for 2 hours, then cooled and concentrated. The residue was dissolved in water (30 ml) and potassium carbonate added in portions. This mixture was extracted 3 times with ethyl acetate. The combined organic extracts were washed with brine, dried over magnesium sulfate and concentrated. The residue was purified by chromatography on silica gel (eluent: hexane/diethyl ether 0-50%) to give the product as colourless oil (0.73 g, 59% yield).

¹H-NMR (400 MHz, CDCl₃): 3.75 (s, 3H, CH₃), 4.55 (s, 2H, CH₂), 4.78 (d, 2H, CH₂), 4.74 (dd, 1H, CH), 4.95 (dd, 1H, CH).

### Example I8

### Preparation of 1,4-dimethyl-3-(2,2,2-trifluoro-ethoxy)-5-trifluoromethyl-1H-pyrazole

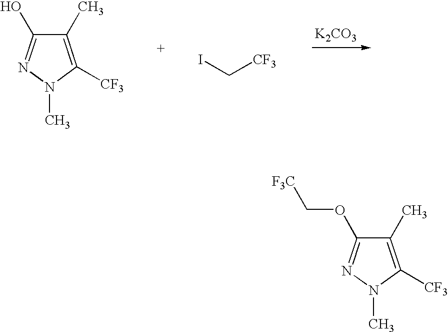

To a solution of 1,4-dimethyl-5-trifluoromethyl-1H-pyrazol-3-ol (3.0 g, 16.67 mmol) in DMF (90 ml), was added trifluoroethyl iodide (3.3 ml, 33.3 mmol) and potassium carbonate (4.6 g, 33.3 mmol). The mixture was stirred at room temperature for 3 days. More trifluoroethyl iodide (3.3 ml, 33.3 mmol) was added and the mixture stirred at room temperature for 2 days. More trifluoroethyl iodide (10 ml, 100 mmol) was added and the mixture stirred at room temperature for 2 days. The reaction mixture was quenched with water and extracted with ethyl acetate. The organic extract was washed with brine, dried over magnesium sulfate and concentrated (809 mg, 16% yield).

### Example I9

### Preparation of 4-bromomethyl-1-methyl-3-(2,2,2-trifluoro-ethoxy)-5-trifluoromethyl-1H-pyrazole

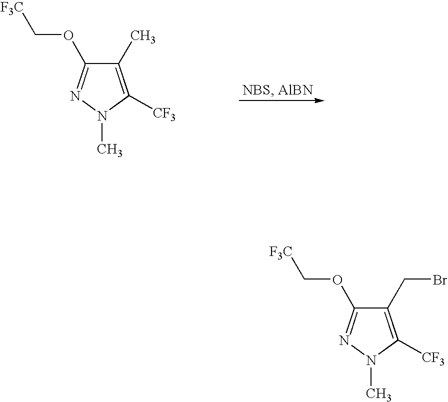

To a solution of 1,4-dimethyl-3-(2,2,2-trifluoro-ethoxy)-5-trifluoromethyl-1H-pyrazole (809 mg, 3.08 mmol) (see Example I8) in carbon tetrachloride (10 ml), were added N-bromosuccinimide (NBS) (712 mg, 4.0 mmol) and azobisisobutyronitrile (AIBN) (50 mg, 3.08 mmol) under nitrogen. The mixture was stirred at room temperature and irradiated with a UV lamp. Then the mixture refluxed with the heat of the lamp. After 30 minutes the mixture was filtered and the solid was washed with dichloromethane. The chlorinated filtrate was concentrated. The residue was triturated with 4:1 hexane/ethyl acetate (50 ml) and the solid gained in this fashion was purified by chromatography (eluent 20%-50% ethyl acetate-hexane) to give the product (715 mg, 68% yield).

### Example I10

### Preparation of 4-(1-hydroxyethyl)-1-methyl-5-(2,2,2-trifluoroethoxy)-3-trifluoromethyl-1H-pyrazole

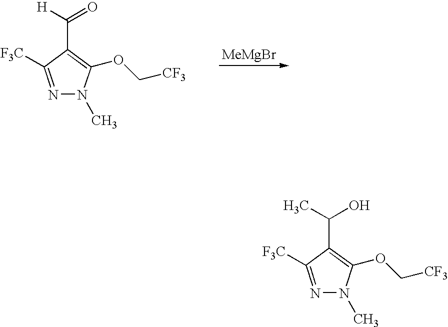

A solution of 1-methyl-5-(2,2,2-trifluoroethoxy)-3-trifluoromethyl-1H-pyrazole-4-carbaldehyde (see Example I1) (5.0 g, 18.1 mmol) in dry diethyl ether (15 ml) was added dropwise to a solution of methyl magnesium bromide (3.0M in diethyl ether) (6.29 ml, 18.8 mmol) in dry diethyl ether (15 ml) at 0° C. The reaction mixture was stirred for 15 minutes, then the reaction was quenched by addition of a cold saturated aqueous ammonium chloride and extracted with diethyl ether (3×50 ml). The combined organic extracts were washed successively with a saturated aqueous ammonium chloride, water and brine, dried over magnesium sulfate and concentrated. Trituration of the residue with hexane gave 4-(2-hydroxyethyl)-1-methyl-5-(2,2,2-trifluoroethoxy)-3-trifluoromethyl-1H-pyrazole as a white solid (4.13 g, 78% yield)

¹H-NMR (400 MHz, CDCl₃): 1.6 (bs, 1H, CH), 1.54 (d, 3H, CH₃), 3.77 (s, 3H, CH₃), 4.7 (m, 2H, CH₂), 5.1 (q, 1H, CH).

### Example I11

### Preparation of 4-(1-bromoethyl)-1-methyl-5-(2,2,2-trifluoroethoxy)-3-trifluoromethyl-1H-pyrazole

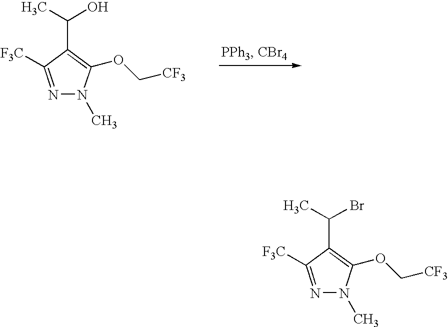

A solution of carbon tetrabromide (5.0 g, 15.5 mmol) in dry dichloromethane (5 ml) was added dropwise to a solution of 4-(2-hydroxyethyl)-1-methyl-5-(2,2,2-trifluoroethoxy)-3-trifluoromethyl-1H-pyrazole (4.0 g, 13.6 mmol) (see Example I10) and triphenyl phosphine (4.1 g, 15.5 mmol) in dry dichloromethane (50 ml) at 0° C. The reaction mixture was stirred for 2 hours at 0° C. and then concentrated. Hexane/ethyl acetate (9:1) was added to the residue and then filtered to remove triphenyl phosphine oxide. The organic extract was concentrated to give 4-(1-bromoethyl)-1-methyl-5-(2,2,2-trifluoroethoxy)-3-trifluoromethyl-1H-pyrazole as a pale yellow oil (7.3 g) which was used without purification.

¹H-NMR (400 MHz, CDCl₃): 2.05 (s, 3H, CH₃), 3.8 (d, 3H, CH₃), 4.43 (q, 1H, CH), 4.55-4.85 (m, 2H, CH₂).

### Example I12

### Preparation of 5-ethylsulfanyl-1-methyl-3-trifluoromethyl-1H-pyrazole-4-carbaldehyde

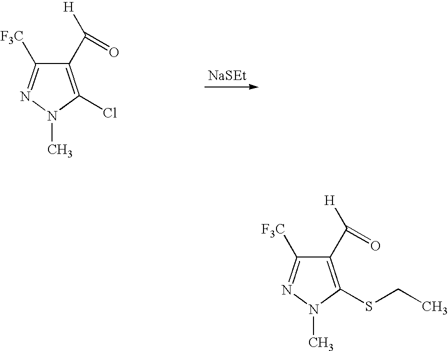

Sodium ethylthiolate (1.05 g, 12.5 mmol) was stirred in dry DMF (50 ml). After 10 minutes 5-chloro-1-methyl-3-trifluoromethyl-1H-pyrazole-4-carbaldehyde (2.12 g, 10 mmol) was added in dry DMF (5 ml). The mixture was stirred at room temperature overnight. More sodium ethylthiolate was added (700 mg, 8.3 mmol) and the reaction mixture stirred at room temperature for 24 hours. The reaction mixture was diluted with water and extracted with diethyl ether (2×). The combined organic extracts were washed (4×) with water, brine, and dried over sodium sulfate and concentrated. The residue was purified by chromatography over silica gel (eluent 0-50% ethyl acetate in hexane) to give the product as a mobile oil (1.228 g, 51% yield).

¹H-NMR (400 MHz, CDCl₃): 1.25 (t, 3H, CH₃), 3.0 (q, 2H, CH₂), 4.05 (s, 3H, CH₃), 10.05 (s, 1H, CH).

### Example I13

### Preparation of 5-ethanesulfinyl-1-methyl-3-trifluoromethyl-1H-pyrazole-4-carbaldehyde

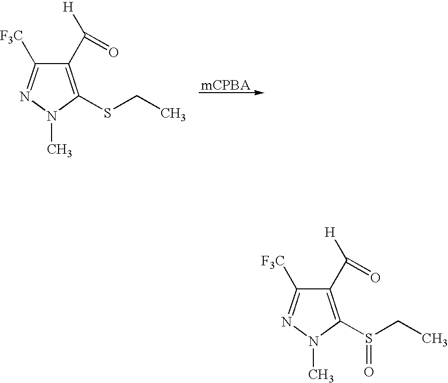

5-Ethylsulfanyl-1-methyl-3-trifluoromethyl-1H-pyrazole-4-carbaldehyde (1.136 g, 4.77 mmol) (see Example I12) was dissolved in dry dichloromethane (30 ml) and cooled to 0° C. 3-Chloroperoxybenzoic acid (MCPBA) (70% by weight) (1.173 g, 4.77 mmol) was added over 10 minutes. The mixture was stirred at room temperature for 16 hours. The reaction was quenched with sodium metabisulfite and the phases separated. The organic phase was extracted (2×) with aqueous sodium hydrogencarbonate, dried over sodium sulfate and concentrated. The residue was purified by chromatography over silica gel (eluent 0-10% methanol in dichloromethane) to give the product as a clear oil (375 mg, 31% yield).

¹H-NMR (400 MHz, CDCl₃): 1.43 (t, 3H, CH₃), 3.26 (m, 2H, CH₂), 4.31 (s, 3H, CH₃), 9.98 (s, 1H, CH).

### Example I14

### Preparation of (5-ethanesulfinyl-1-methyl-3-trifluoromethyl-1H-pyrazol-4-yl)-methanol

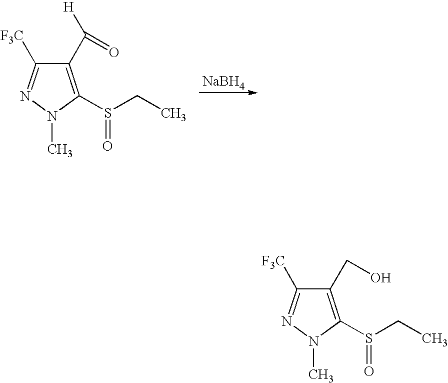

5-Ethanesulfinyl-1-methyl-3-trifluoromethyl-1H-pyrazole-4-carbaldehyde (370 mg, 1.55 mmol) (see Example I13) was dissolved in methanol (10 ml) and cooled to 0° C. Sodium borohydride (30 mg, 0.75 mmol) was added and the mixture stirred at room temperature for 1 hour. The reaction was cooled to 0° C. and quenched with water. The mixture was stirred at room temperature and extracted with ethyl acetate (2×). The combined organic extracts were dried over sodium sulfate and concentrated to give the product as an oil (380 mg, 96% yield).

¹H-NMR (400 MHz, CDCl₃): 1.48 (t, 3H, CH₃), 3.18-3.48 (m, 2H, CH₂), 4.05 (s, 3H, CH₃), 4.8 (s, 2H, CH₂).

### Example I15

### Preparation of 4-bromomethyl-5-ethylsulfanyl-1-methyl-3-trifluoromethyl-1H-pyrazole

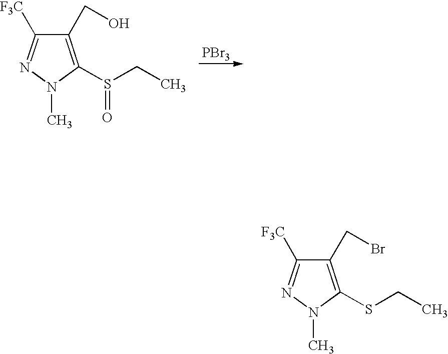

(5-Ethanesulfinyl-1-methyl-3-trifluoromethyl-1H-pyrazol-4-yl)-methanol (375 mg, 1.46 mmol) (see Example I14) was dissolved in diethyl ether (10 ml) and a drop of pyridine was added. The solution was cooled to 0° C. and phosphorous tribromide (0.07 ml, 0.78 mmol) was added. The reaction mixture was stirred at room temperature for 1 hour. The reaction mixture was cooled to 0° C. before quenching with aqueous sodium hydrogencarbonate. The mixture was diluted with diethyl ether and water and the layers were separated. The aqueous layer was extracted with diethyl ether and the combined organic extracts were washed with brine, dried over sodium sulfate and concentrated to give the product as a semisolid (280 mg, 63% yield). The bromination reduced the sulfoxide to the sulfide.

¹H-NMR (400 MHz, CDCl₃): 1.28 (t, 3H, CH₃), 2.87 (q, 2H, CH₂), 4.01 (s, 3H, CH₃), 4.55 (s, 2H, CH₂).

This intermediate was reacted according to Example P7 to yield Compound No. 1.076 of Table 32.

Furthermore, the 5-ethylsulfonyl compounds were prepared by reacting 5-ethylsulfanyl-1-methyl-3-trifluoromethyl-1H-pyrazole-4-carbaldehyde with two equivalents of 3-chloroperoxybenzoic acid (MCPBA) according to Example I13 to give the 5-ethylsulfonyl compound, reducing the aldehyde according to Example I14, brominating the alcohol according to Example I15 (in which the 5-ethylsulfonyl remains intact), and then coupling the bromide according to Example P7 to yield Compound No. 1.079 of Table 32.

Similarly, the 5-methylsulfanyl compounds were prepared by reacting 5-chloro-1-methyl-3-trifluoromethyl-1H-pyrazole-4-carbaldehyde with sodium methylthiolate according to Example I12, reducing the aldehyde according to Example I14, brominating the alcohol according to Example I15, and then coupling the bromide according to Example P7 to yield Compound No. 1.088 of Table 32.

### Example P1

### Preparation of 5-bromo-2-[1-methyl-5-(2,2,2-trifluoroethoxy)-3-trifluoromethyl-1H-pyrazol-4-ylmethylsulfanyl]-thiazole

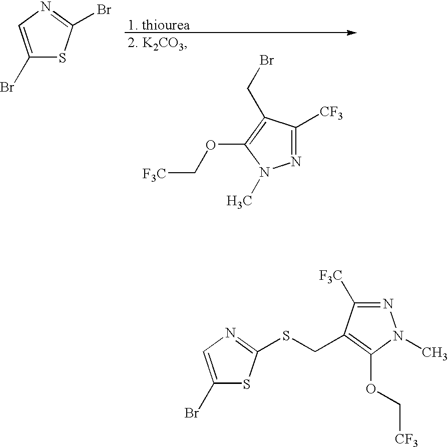

Thiourea (86 mg, 1.05 mmol) was added to a solution of 2,5-dibromothiazole (243 mg, 1 mmol) in ethanol (10 ml) at room temperature. The solution was heated under reflux for 2.5 hours. The reaction mixture was cooled to room temperature. To the reaction mixture was added 4-bromomethyl-1-methyl-5-(2,2,2-trifluoroethoxy)-3-trifluoromethyl-1H-pyrazole (341 mg, 1 mmol) (see Example I3 or Example I4), followed by potassium carbonate (179 mg, 1.3 mmol). The mixture was heated under reflux for 30 minutes, then cooled and stored at room temperature for 16 hours. The reaction mixture was filtered and the solid washed with ethyl acetate. The combined organic phases were concentrated and the residue purified by chromatography on silica gel (eluent 0-50% ethyl acetate in hexane) to yield Compound No. 1.001 of Table 32 as a colourless oil (448 mg, 98% yield).

The following compounds were also prepared according to this procedure: Compound No. 1.004 of Table 32, Compound No. 1.007 of Table 32, Compound No. 1.010 of Table 32, Compound No. 1.013 of Table 32, Compound No. 1.021 of Table 32, Compound No. 1.024 of Table 32, Compound No. 1.027 of Table 32, Compound No. 1.073 of Table 32 and Compound No. 1.082 of Table 32.

### Example P2

### Preparation of 5-bromo-2-[1-methyl-5-(2,2,2-trifluoroethoxy)-3-trifluoromethyl-1H-pyrazol-4-ylmethanesulfinyl]-thiazole

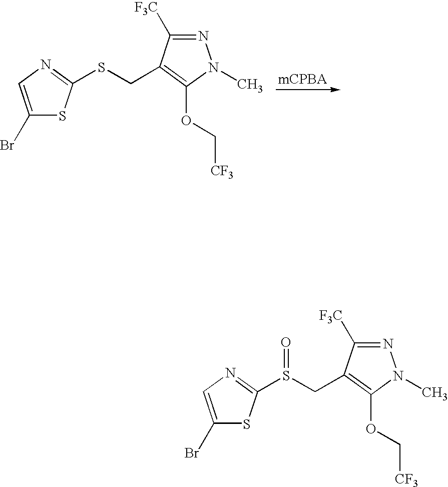

3-Chloroperoxybenzoic acid (70% by weight) (119 mg, 0.47 mmol) was added to a solution of 5-bromo-2-[1-methyl-5-(2,2,2-trifluoroethoxy)-3-trifluoromethyl-1H-pyrazol-4-ylmethylsulfanyl]-thiazole (see Example P1) (220 mg, 0.48 mmol) in dichloromethane (10 ml) at 0° C. The mixture was stirred at 0° C. for 2.5 hours. The reaction mixture was diluted with dichloromethane (25 ml) and washed successively with sodium bicarbonate, sodium carbonate, water and brine. The organic extract was dried over magnesium sulfate and concentrated. The residue was washed with a small amount of hexane to yield Compound No. 1.002 of Table 32 as a white solid (179 mg, 79% yield).

### Example P3

### Preparation of 5-bromo-2-[1-methyl-5-(2,2,2-trifluoroethoxy)-3-trifluoromethyl-1H-pyrazol-4-ylmethanesulfonyl]-thiazole

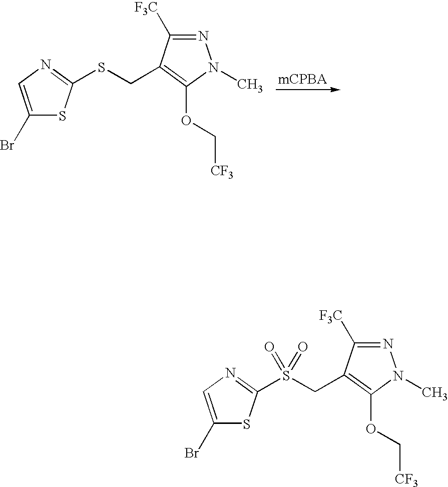

3-Chloroperoxybenzoic acid (70% by weight) (321 mg, 1.3 mmol) was added to a solution of 5-bromo-2-[1-methyl-5-(2,2,2-trifluoroethoxy)-3-trifluoromethyl-1H-pyrazol-4-ylmethylsulfanyl]-thiazole (see Example P1) (220 mg, 0.48 mmol) in dichloromethane (10 ml) at 0° C. The mixture was stirred at 0° C. for 2.5 hours and then at room temperature for 16 hours. The reaction mixture was diluted with dichloromethane (25 ml) and washed successively with sodium bicarbonate, sodium carbonate, water and brine. The organic extract was dried over magnesium sulfate and concentrated. The residue was washed with a small amount of hexane to yield Compound No. 1.003 of Table 32 as a white solid (210 mg, 89% yield).

The following compounds were also prepared according to methods in Example P1, Example P2 and Example P3:

### Compound No. 1.006 of Table 32, Compound No. 1.009 of Table 32, Compound No. 1.012 of Table 32 and Compound No. 1.015 of Table 32.

### Example P4

### Preparation of 5-bromo-2-(5-chloro-1-methyl-3-trifluoromethyl-1H-pyrazol-4-ylmethylsulfanyl)-thiazole

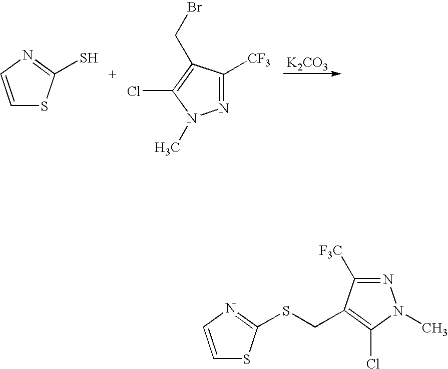

4-Bromomethyl-5-chloro-1-methyl-3-trifluoromethyl-1H-pyrazole (2.4 g, 8 mmol) (see Example I4) was added to a solution of 2-mercaptothiazole (936 mg, 8 mmol) in dry acetonitrile (20 ml), followed by potassium carbonate (1.24 g, 9 mmol). The reaction mixture was heated under reflux for 2 hours, and then allowed to cool to room temperature. The solid was removed by filtration and washed with acetonitrile. The combined organic phases were concentrated and the residue was purified by chromatography on silica gel (eluent 0-50% ethyl acetate in hexane) to yield Compound No. 1.020 of Table 32 as a colourless oil (2.40 g, 98% yield).

The following compound was also prepared according to this procedure:

### Compound No. 1.047 of Table 32.

### Example P5

### Preparation of 2-[1-methyl-5-(2,2,2-trifluoroethoxy)-3-trifluoromethyl-1H-pyrazol-4-ylmethanesulfonyl]-4-trifluoromethyl-thiazole-5-carboxylic acid ethyl ester

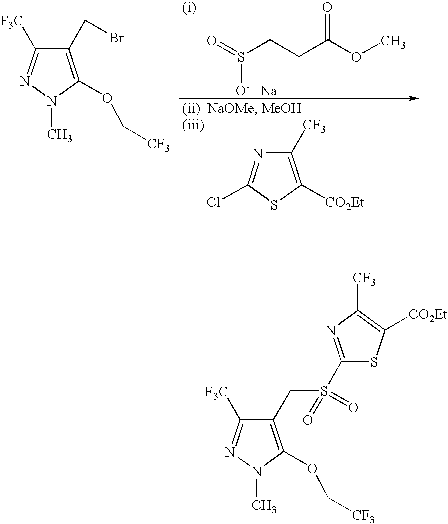

To a solution of 4-bromomethyl-1-methyl-5-(2,2,2,-trifluoroethoxy)-3-trifluoromethyl-1H-pyrazole (0.21 g, 0.62 mmol) (see Example I3 or Example I4) in dimethylsulfoxide (DMSO) (0.9 ml) was added 3-methoxy-3-oxopropane-1-sulfinate (0.11 g, 0.63 mmol) (prepared according to Tetrahedron Letters 2002 (43) 8479) in one portion with external cooling in a water bath. The reaction was stirred at room temperature for 16 hours. A solution of sodium methoxide in methanol (25% weight/volume) (0.14 ml, 0.61 mmol) was added gradually with stirring and external cooling in a water bath. The reaction mixture was stirred at room temperature for 15 minutes and then 2-chloro-4-trifluoromethyl-thiazole-5-carboxylic acid ethyl ester (0.16 g, 0.61 mmol) was added. The reaction was stirred at room temperature for 16 hours. The reaction mixture was extracted with ethyl acetate (3 ml) and brine (4 ml). The organic extract was separated and the aqueous phase extracted with ethyl acetate (2×4 ml). The combined organic extracts were washed with brine (3×4 ml), dried over magnesium sulfate and filtered. The filtrate was concentrated and the residue purified by chromatography on silica gel (eluent 0-80% diethyl ether in hexane) to afford Compound No. 1.019 of Table 32 as a colourless oil (0.16 g, 47% yield).

### Example P6

### Alternative preparation of 5-bromo-2-(5-chloro-1-methyl-3-trifluoromethyl-1H-pyrazol-4-ylmethanesulfonyl)-thiazole

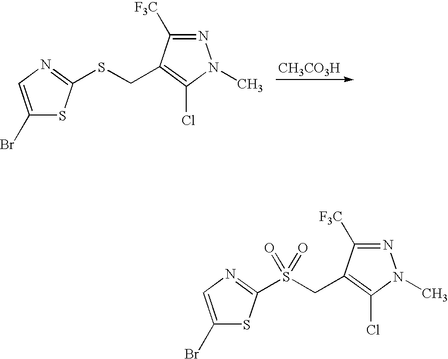

To a solution of 5-bromo-2-(5-chloro-1-methyl-3-trifluoromethyl-1H-pyrazol-4-ylmethylsulfanyl)-thiazole (1.69 g, 4.26 mmol) (see Example P1) in dichloromethane (15 ml) was added peracetic acid in acetic acid (35% by weight) (2 ml). The mixture was heated under reflux for 3 hours, and then allowed to cool to room temperature. Dichloromethane (50 ml) was added and the reaction mixture was washed successively with water, aqueous sodium metabisulfite, aqueous sodium hydrogencarbonate and brine. The organic extract was dried over magnesium sulfate and concentrated. The residue was triturated with hexane to give a white solid. The solid was dissolved in dichloromethane/1,2-dichloroethane (1:1) and peracetic acid in acetic acid (35% by weight) (1 ml) was added. The reaction mixture was heated under reflux for 60 minutes and then cooled to room temperature. Dichloromethane (50 ml) was added and the reaction mixture was washed successively with water, aqueous sodium metabisulfite, aqueous sodium hydrogencarbonate and brine. The organic extract was dried over magnesium sulfate and concentrated. The solid was triturated with hexane to give Compound No. 1.006 of Table 32 as a white solid (1.70 g, 93% yield).

The methods used in Example P3 (two equivalents of 3-chloroperoxybenzoic acid, MCPBA) and Example P6 (two equivalents of peracetic acid) are equally useful in the preparation of sulfones from sulfides. Similarly, the method used in Example P2 (one equivalent of 3-chloroperoxybenzoic acid, MCPBA) and the use of one equivalent peracetic acid (no example given) are equally useful in the preparation of sulfoxides from sulfides or sulfones from sulfoxides.

Also Compound No. 1.088 of Table 32 was oxidised with one equivalent of 3-chlorperoxybenzoic acid (MCPBA) to give Compound No. 1.089 of Table 32. Compound No. 1.088 of Table 32 was oxidised with two equivalents of MCPBA to give Compound No. 1.090 of Table 32 and Compound No. 1.091 of Table 32. Compound No. 1.088 of Table 32 was oxidised with three equivalents of MCPBA to give Compound No. 1.092 of Table 32. Compound No. 1.088 of Table 32 was oxidised with four equivalents of MCPBA to give Compound No. 1.093 of Table 32.

Compound No. 1.076 of Table 32 was oxidised with two equivalents of 3-chloroperoxybenzoic acid (MCPBA) to give Compound No. 1.078 of Table 32 as a mixture of diastereoisomers and also as a by-product some compound No. 1.080 of Table 32. And Compound No. 1.076 of Table 32 was oxidised with four equivalents of MCPBA to give Compound No. 1.081 of Table 32.

### Example I16

### Preparation of 5-chloro-2-methanesulfonyl-thiazole

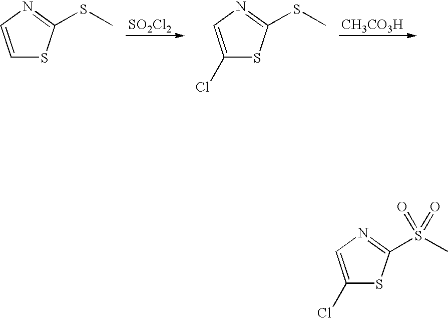

A solution of sulfuryl chloride (15.5 g, 115 mmol) in dry dichloromethane (20 ml) was added dropwise to a stirred solution of 2-(methylthio)thiazole (15 g, 115 mmol) in dry dichloromethane (100 ml) at 0° C. The reaction mixture was stirred for 1 hour at 0° C. The reaction was quenched by the addition of ice/water. The phases were separated and the aqueous phase was extracted with dichloromethane. The combined organic extracts were washed successively with water and brine, dried over magnesium sulfate and concentrated. The residue was purified by chromatography on silica gel (eluent 10% ether in hexane) to give 5-chloro-2-(methylthio)thiazole as a colourless oil (13.9 g, 74% yield). This compound (13.9 g, 84 mmol) was dissolved in dichloromethane (100 ml) and cooled to −10° C. Peracetic acid (36-40% in acetic acid, 35 ml, 187 mmol) was added dropwise, keeping the temperature between −5° C. and −10° C. The reaction mixture was allowed to warm to room temperature and stirred for 16 hours at room temperature and then 2 hours at reflux. The reaction mixture was diluted with dichloromethane (100 ml), then washed successively with water (2×50 ml), sodium hydrogencarbonate (50 ml), sodium metabisulfite, water and brine, dried over magnesium sulfate and concentrated to give 5-chloro-2-methanesulfonyl-thiazole (15.0 g, 90% yield).

¹H-NMR (400 MHz, CDCl₃): 3.31 (s, 3H, CH₃), 7.83 (s, 1H, CH).

### Example P7

### Preparation of 5-chloro-2-(5-difluoromethoxy-1-methyl-3-trifluoromethyl-1H-pyrazol-4-ylmethylsulfanyl)-thiazole

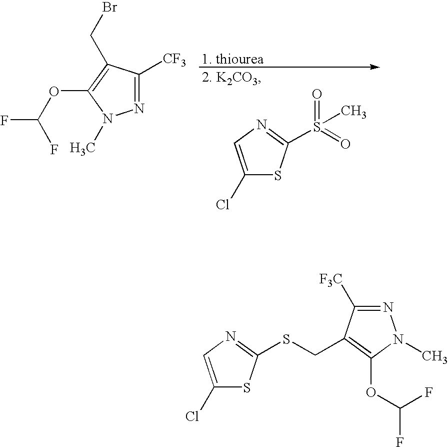

Thiourea (505 mg, 6.47 mmol) was added to a solution of 4-bromomethyl-5-difluoromethoxy-1-methyl-3-trifluoromethyl-1H-pyrazole (2.0 g, 6.47 mmol) (prepared according to WO 04/013106) in ethanol (20 ml) at room temperature. The solution was heated under reflux for 1 hour and then cooled to room temperature. To the reaction mixture was added 5-chloro-2-methylsulfonyl-thiazole (1.27 g, 6.47 mmol) (see Example I16) followed by potassium carbonate (179 mg, 1.3 mmol). The mixture was heated under reflux for 2 hours and then cooled to room temperature. The reaction was quenched by the addition of water (70 ml) and the mixture extracted with ethyl acetate (3×40 ml). The combined organic extracts were washed successively with dilute aqueous citric acid, water and brine, dried over magnesium sulfate and concentrated. The residue was purified by chromatography on silica gel (eluent 0-50% ethyl acetate in hexane) to give Compound No. 1.53 of Table 32 as a colourless oil (1.96 g, 80% yield).

The following compounds were also prepared according to this procedure: Compound No. 1.056 of Table 32, Compound No. 1.059 of Table 32, Compound No. 1.060 of Table 32, Compound No. 1.061 of Table 32, Compound No. 1.097 of Table 32, Compound No. 1.102 of Table 32, Compound No. 1.105 of Table 32, Compound No. 1.112 of Table 32, Compound No. 1.115 of Table 32, Compound No. 1.118 of Table 32, Compound No. 1.126 of Table 32, Compound No. 1.131 of Table 32 and Compound No. 1.134 of Table 32.

### Example I17

### Preparation of 5-difluoromethyl-2-methanesulfonyl-thiazole

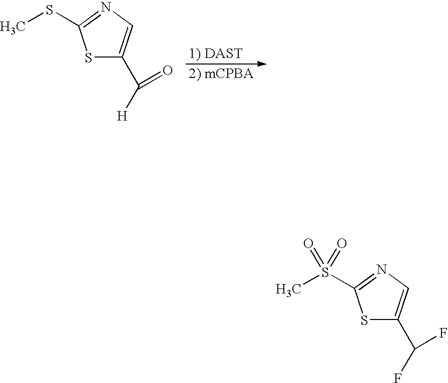

To a solution of 2-methylsulfanyl-thiazole-5-carbaldehyde (1.48 g, 9.3 mmol) (prepared according to EP 301613) in dichloromethane (60 ml) was added (diethylamino)sulfur trifluoride (DAST) (3.74 g, 23.2 mmol). The reaction was stirred at room temperature for 2 hours. The reaction was quenched with aqueous sodium hydrogencarbonate and extracted three times with dichloromethane. The combined organic extracts were washed with brine and dried over magnesium sulfate. 3-Chloroperoxybenzoic acid (MCPBA) (60% by weight) (8.0 g, 28 mmol) was added to the solution. The reaction was stirred at room temperature for 2 hours and then was quenched with aqueous sodium metabisulfite (20% by weight in water). The mixture was extracted with dichloromethane and the combined organic extracts were washed with aqueous sodium hydrogencarbonate and brine and concentrated. The residue was purified by chromatography on silica gel (eluent 0-50% ethyl acetate in hexane) to give the product as yellow solid (360 mg, 18% yield).

¹H-NMR (400 MHz, CDCl₃): 3.04 (s, 3H, CH₃), 6.95 (t, 1H, CH), 8.09 (s, 1H, CH).

### Example P8

### Preparation of 5-difluoromethyl-2-[1-methyl-5-(2,2,2-trifluoro-ethoxy)-3-trifluoromethyl-1H-pyrazol-4-ylmethylsulfanyl]-thiazole

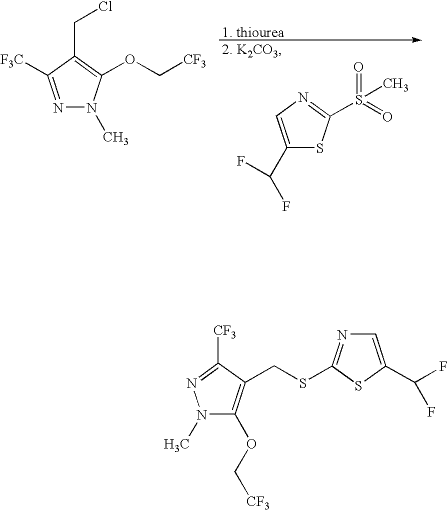

Thiourea (129 mg, 1.7 mmol) was added to a solution of 4-chloromethyl-1-methyl-5-(2,2,2-trifluoroethoxy)-3-trifluoromethyl-1H-pyrazole (0.42 g, 1.4 mmol) (see Example I5) in ethanol (20 ml) at room temperature and stirred for one hour. To the reaction mixture was added 5-difluoromethyl-2-methanesulfonyl-thiazole (0.3 g, 1.4 mmol) (see Example I17) followed by potassium carbonate (396 mg, 2.8 mmol). The mixture was heated under reflux for 2 hours and then cooled to room temperature. The reaction was quenched by the addition of water (20 ml) and the mixture extracted with ethyl acetate (3×20 ml). The combined organic extracts were washed with brine, dried over magnesium sulfate and concentrated. The residue was purified by chromatography on silica gel (eluent 0-50% ethyl acetate in hexane) to give Compound No. 1.085 of Table 32 (353 mg, 59% yield).

### Example I18

### Preparation of 5-bromo-2-p-tolylsulfanylthiazole

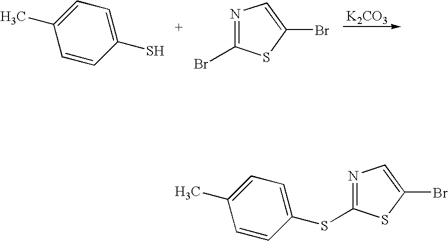

4-Methylbenzenethiol (10.2 g, 82 mmol) and 2,4-dibromothiazole (20 g, 82 mmol) were dissolved in acetonitrile (150 ml) and potassium carbonate (12.5 g, 90.5 mmol) was added. The mixture was heated under reflux for 2 hours, cooled to room temperature and filtered through Celite®. The filtrate was concentrated and the residue purified by chromatography on silica gel (eluent 0-50% ethyl acetate in hexane). The product was isolated as orange oil which crystallised upon standing (19 g, 80.9% yield).

¹H-NMR (400 MHz, CDCl₃): 2.40 (s, 3H, CH₃), 7.25 (d, 2H, 2×CH), 7.53 (m, 3H, 3×CH).

### Example I19

### Preparation of 2,2,2-trifluoro-1-(2-p-tolylsulfanyl-thiazol-5-yl)-ethanone

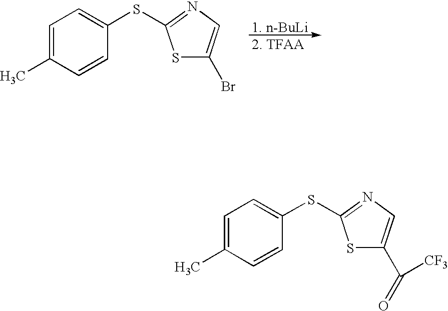

n-Butyl lithium (2.5M in hexane) (1 ml, 2.5 mmol) was added dropwise to a solution of 5-bromo-2-p-tolylsulfanyl-thiazole (576 mg, 2 mmol) (see Example I18) in dry THF (10 ml) at −78° C. under nitrogen. After 10 minutes trifluoroacetic anhydride (TFAA) (0.3 ml, 2.2 mmol) was added dropwise. The reaction was stirred at −78° C. for 1 hour and then quenched with saturated aqueous ammonium chloride at −78° C. The mixture was allowed to warm to room temperature and extracted three times with ethyl acetate. The combined organic extracts were washed with brine, dried over magnesium sulfate and concentrated. The residue was purified by chromatography on silica gel (eluent 0-50% ethyl acetate in hexane) to give the product as an orange solid (290 mg, 47.8% yield).

¹H-NMR (400 MHz, CDCl₃): 2.41 (s, 3H, CH₃), 7.27 (d, 2H, 2×CH), 7.52-7.56 (m, 3H, 3×CH).

### Example I20

### Preparation of 2,2,2-trifluoro-1-(2-p-tolylsulfonyl-thiazol-5-yl)-ethanone

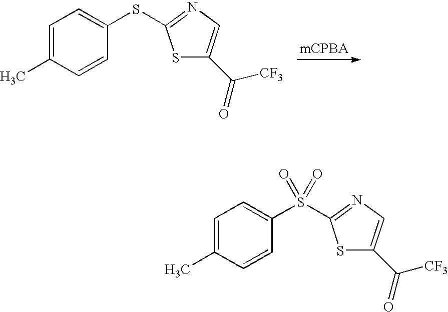

3-Chloroperoxybenzoic acid (60% by weight) (346 mg, 2.0 mmol) was added to a solution of 2,2,2-trifluoro-1-(2-p-tolylsulfanyl-thiazol-5-yl)-ethanone (290 mg, 0.9 mmol) (see Example I19) in dichloromethane (40 ml). The reaction was stirred at room temperature for 2 hours and then quenched with aqueous sodium metabisulfite. The phases were separated and the organic extract washed with aqueous sodium carbonate and brine, dried over magnesium sulfate and then concentrated to give the product as yellow solid (300 mg, 99% yield).

¹H-NMR (400 MHz, CDCl₃): 2.40 (s, 3H, CH₃), 7.25 (d, 2H, 2×CH), 7.53 (d, 2H, 2×CH), 7.84 (s, 1H, CH).

### Example P9

### Preparation of 2,2,2-Trifluoro-1-{2-[1-methyl-5-(2,2,2-trifluoro-ethoxy)-3-trifluoromethyl-1H-pyrazol-4-ylmethylsulfanyl]-thiazol-5-yl}-ethanone

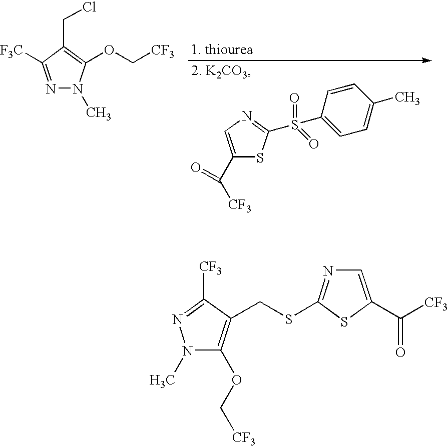

Thiourea (160 mg, 2.1 mmol) was added to a solution of 4-chloromethyl-1-methyl-5-(2,2,2-trifluoroethoxy)-3-trifluoromethyl-1H-pyrazole (0.59 g, 2.0 mmol) (see Example I5) in acetonitrile (60 ml) at room temperature and stirred for one hour. To the reaction mixture was added 2,2,2-trifluoro-1-(2-p-tolylsulfonyl-thiazol-5-yl)-ethanone (0.55 g, 2.1 mmol) (see Example I20) followed by potassium carbonate (847 mg, 6.0 mmol). The mixture was heated under reflux for 2 hours and then cooled to room temperature. The reaction was quenched by the addition of water (20 ml) and the mixture extracted with ethyl acetate (3×20 ml). The combined organic extracts were washed with brine, dried over magnesium sulfate and concentrated. The residue was purified by chromatography on silica gel (eluent 0-50% ethyl acetate in hexane) to give Compound No. 1.100 of Table 32 (383 mg, 39% yield).

### Example I21

### Preparation of 2-p-tolylsulfanyl-thiazole-5-carboxylic acid tert-butylamide

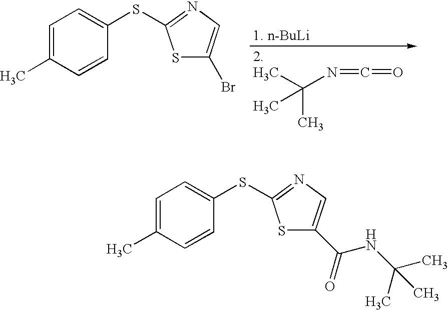

n-Butyl lithium (2.5M in hexane) (1.68 ml, 4.2 mmol) was added dropwise to a solution of 5-bromo-2-p-tolylsulfanyl-thiazole (1.0 g, 3.5 mmol) (see Example I18) in dry THF (10 ml) at −78° C. under nitrogen. After 10 minutes tert-butyl isocyanate (0.48 ml, 4.2 mmol) was added dropwise. The reaction was stirred at −78° C. for 1 hour and then quenched with saturated aqueous ammonium chloride at −78° C. The mixture was allowed to warm to room temperature and extracted three times with ethyl acetate. The combined organic extracts were washed with brine, dried over magnesium sulfate and concentrated. The residue was purified by chromatography on silica gel (eluent 0-50% ethyl acetate in hexane) to give the product as an orange solid (1.0 g, 93% yield).

¹H-NMR (400 MHz, CDCl₃): 1.40 (s, 9H, 3×CH₃), 2.41 (s, 3H, CH₃), 5.54 (bs, 1H, NH), 7.27 (d, 2H, 2×CH), 7.56 (d, 2H, 2×CH), 7.84 (s, 1H, CH).

### Example I22

### Preparation of 2-p-tolylsulfonyl-thiazole-5-carboxylic acid tert-butylamide

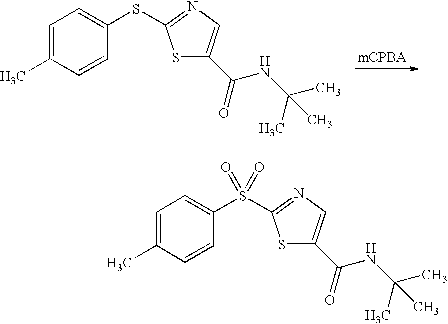

3-Chloroperoxybenzoic acid (60% by weight) (2.6 g, 9.2 mmol) was added to a solution of 2-p-tolylsulfanyl-thiazole-5-carboxylic acid tert-butylamide (1.1 g, 3.7 mmol) (see Example I21) in dichloromethane (10 ml). The reaction was stirred at room temperature for 2 hours and then quenched with aqueous sodium metabisulfite. The phases were separated and the organic extract was washed with aqueous sodium carbonate and brine, dried over magnesium sulfate and then concentrated to give the product as yellow solid (1.2 g, 96% yield).

¹H-NMR (400 MHz, CDCl₃): 1.45 (s, 9H, 3×CH₃), 2.44 (s, 3H, CH₃), 7.37 (d, 2H, 2×CH), 7.96 (d, 2H, 2×CH), 8.08 (s, 1H, CH).

### Example P10

### Preparation of 2-[1-methyl-5-(2,2,2-trifluoro-ethoxy)-3-trifluoromethyl-1H-pyrazol-4-ylmethylsulfanyl]-thiazole-5-carboxylic acid tert-butylamide

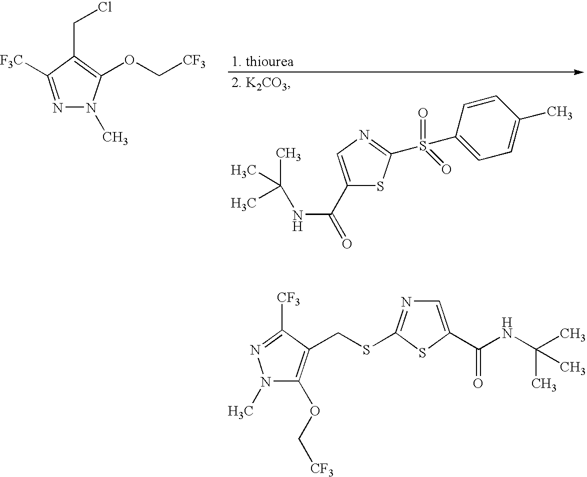

Thiourea (225 mg, 2.96 mmol) was added to a solution of 4-chloromethyl-1-methyl-5-(2,2,2-trifluoroethoxy)-3-trifluoromethyl-1H-pyrazole (0.8 g, 2.7 mmol) (see Example I5) in ethanol (100 ml) at room temperature and stirred for one hour. To the reaction mixture was added 2-p-tolylsulfonyl-thiazole-5-carboxylic acid tert-butylamide (1.0 g, 2.96 mmol) (see Example I22) followed by potassium carbonate (1.14 g, 8.1 mmol). The mixture was heated under reflux for 2 hours and then cooled to room temperature. The reaction was quenched by the addition of water (20 ml) and the mixture extracted with ethyl acetate (3×20 ml). The combined organic extracts were washed with brine, dried over magnesium sulfate and concentrated. The residue was purified by chromatography on silica gel (eluent 0-50% ethyl acetate in hexane) to give Compound No. 1.142 of Table 32 (320 mg, 25% yield).

### Example I23

### Preparation of 5-trimethylsilyl-2-p-tolylsulfanylthiazole

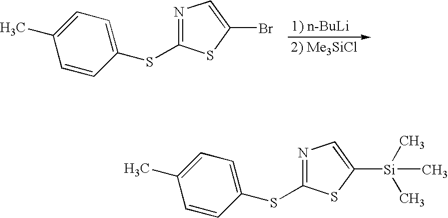

A solution of 5-bromo-2-p-tolylsulfanylthiazole (see Example I18) (0.57 g, 2.0 mmol) in dry tetrahydrofuran (10 ml) was cooled to −78° C. N-Butyl lithium (2.5M in hexane) (0.9 ml, 2.25 mmol) was added dropwise over 10 minutes. The solution was stirred for 15 minutes, then trimethylsilyl chloride (0.24 g, 2.2 mmol) was added dropwise over 15 minutes. The reaction mixture was stirred at −78° C. for 1 hour, then allowed to warm to room temperature. The reaction was quenched by addition of saturated aqueous ammonium chloride and extracted with diethyl ether (3×50 ml). The combined organic extracts were washed successively with water and brine, dried over magnesium sulfate and concentrated. The residue was purified by chromatography on silica gel (eluent 0-40% ethyl acetate in hexane) to give 4-trimethylsilyl-2-p-tolyl-sulfanylthiazole as a colourless oil (0.56 g, 100% yield).

¹H-NMR (400 MHz, CDCl₃): 0.05 (s, 9H, 3×CH₃), 2.25 (s, 3H, CH₃), 7.0-7.4 (m, 4H, CH), 7.4 (s, 1H; CH).

### Example I24

### Preparation of 5-trimethylsilyl-2-p-tolylsulfonylthiazole

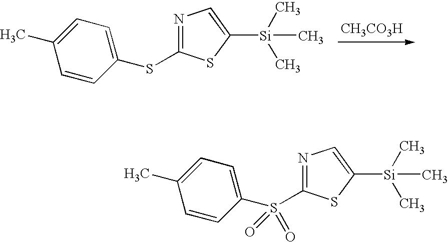

Peracetic acid in acetic acid (35% by weight) (2.5 ml, 12.5 mmol) was added dropwise over 10 minutes to a solution of 4-trimethylsilyl-2-p-tolylsulfanylthiazole (1.65 g, 5.9 mmol) (see Example I23) in dichloromethane (30 ml). The reaction mixture was stirred at room temperature for 16 hours. The reaction was quenched by the addition of dichloromethane (50 ml) and the organic extract was washed successively with aqueous sodium carbonate, aqueous sodium metabisulfite, water and brine. The organic extract was dried over magnesium sulfate and concentrated. The residue was triturated with hexane to give 5-trimethylsilyl-2-p-tolylsulfonylthiazole as a white solid (1.98 g, 100% yield).

¹H-NMR (400 MHz, CDCl₃): 0.35 (s, 9H, 3×CH₃), 2.42 (s, 3H, CH₃), 7.35 (dd, 2H, 2×CH), 8.0 (dd, 2H, 2×CH), 7.9 (s, 1H, CH).

### Example P11

### Preparation of 2-[1-methyl-5-(2,2,2-trifluoro-ethoxy)-3-trifluoromethyl-1H-pyrazol-4-ylmethanesulfanyl]-5-trimethylsilanyl-thiazole

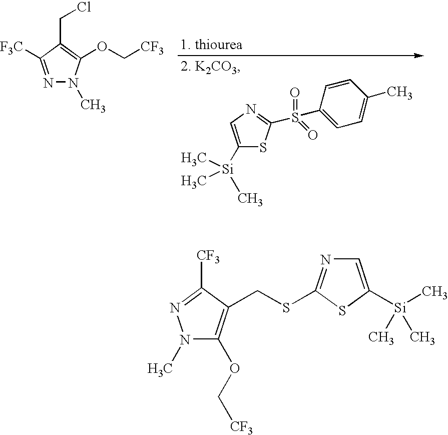

Thiourea (593 mg, 7.6 mmol) was added to a solution of 4-bromomethyl-5-(2,2,2-trifluoro-ethoxy)-1-methyl-3-trifluoromethyl-1H-pyrazole (2.10 g, 7.1 mmol) (see Example I5) in acetonitrile (20 ml). The solution was heated under reflux for 3 hours. To the reaction mixture was added 5-trimethylsilyl-2-p-tolylsulfonylthiazole (1.90 g, 6.10 mmol) (see Example I24), followed by potassium carbonate (1.68 g, 12.2 mmol). The mixture was heated under reflux for 3 hours and then cooled to room temperature. The reaction was quenched by the addition of water (70 ml) and the mixture extracted with ethyl acetate (3×50 ml). The combined organic extracts were washed successively with water and brine, dried over magnesium sulfate and concentrated. The residue was purified by chromatography on silica gel (eluent 0-35% ethyl acetate in hexane) to give Compound No. 1.145 of Table 32 as a colourless oil (1.28 g, 47% yield).

### Example P12

### Preparation of 2-[1-methyl-5-(2,2,2-trifluoro-ethoxy)-3-trifluoromethyl-1H-pyrazol-4-ylmethanesulfonyl]-5-trimethylsilanyl-thiazole

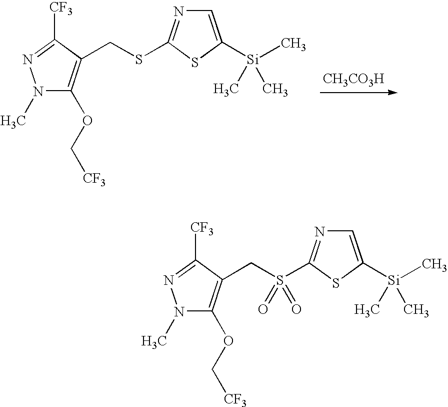

Peracetic acid in acetic acid (35% by weight) (4.0 ml, 20 mmol) was added dropwise over 10 minutes to a solution of 2-[1-methyl-5-(2,2,2-trifluoroethoxy)-3-trifluoromethyl-1H-pyrazol-4-ylmethanesulfanyl]-5-trimethylsilanyl-thiazole (0.3 g, 0.67 mmol) (see Example P11) in dichloromethane (10 ml). The reaction mixture was stirred at room temperature for 60 hours. The reaction was quenched by the addition of dichloromethane (60 ml) and the organic extract was washed successively with aqueous sodium carbonate, aqueous sodium metabisulfite, water and brine. The organic extract was dried over magnesium sulfate and concentrated. The residue was triturated with hexane to give Compound No 1.147 of Table 32 as a white solid (0.31 g, 96% yield).

### Example P13

### Preparation of 5-chloro-2-{1-[1-methyl-5-(2,2,2-trifluoro-ethoxy)-3-trifluoromethyl-1H-pyrazol-4-yl]-ethylsulfanyl}-thiazole

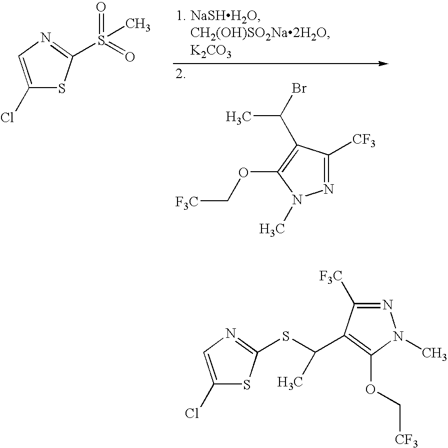

A mixture of the 5-chloro-2-methanesulfonyl-thiazole (1.58 g, 8.0 mmol) (see Example I16), sodium hydrosulfide (0.9 g, 16.0 mmol), hydroxymethanesulfinic acid (2.46 g, 16.0 mmol), potassium carbonate (2.22 g, 16.0 mmol) and dry N,N-dimethylformamide (100 ml) was stirred at 0° C. for 2 hours. 4-(1-Bromoethyl)-5-(2,2,2-trifluoro-ethoxy)-1-methyl-1H-pyrazole (see Example I11) (7.3 g) was added and the reaction mixture was stirred for 16 hours at room temperature. The reaction was quenched by the addition of water (250 ml) and extracted with hexane/ethyl acetate (7:3, 3×100 ml). The combined organic extracts were washed with water (2×) and brine, dried over magnesium sulfate and concentrated. The residue was purified by chromatography on silica gel (eluent 0-40% ethyl acetate in hexane) to give Compound No. 1.094 of Table 32 as a colourless oil (1.77 g, 52% yield).

### Example I25

### Preparation of 3-(5-chloro-thiazole-2-sulfanyl)-propionic acid methyl ester

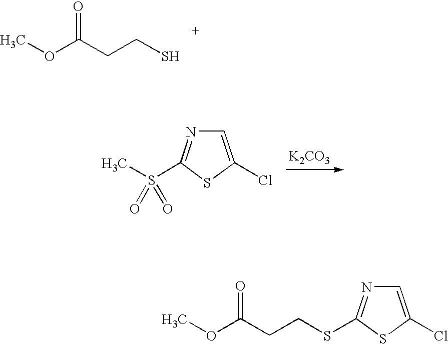

3-Mercapto-propionic acid methyl ester (0.36 g, 3.0 mmol) and 5-chloro-2-methanesulfonyl-thiazole (0.5 g, 2.53 mmol) (see Example I16) were dissolved in dry DMF (3 ml). Potassium carbonate (0.53 g, 3.8 mmol) was added and the reaction was heated to 110° C. for 1 hour. The reaction was cooled to room temperature, water (12 ml) was added and the reaction mixture was extracted several times with ethyl acetate. The combined organic extracts were washed with brine, dried over magnesium sulfate and concentrated. The residue was purified by chromatography on silica gel (eluent 7:3 hexane/diethyl ether) to give the product as a colourless oil (0.35 g, 57% yield).

¹H-NMR (400 MHz, CDCl₃): 2.80 (t, 2H, CH₂), 3.43 (t, 2H, CH₂), 3.71 (s, 3H, CH₃), 7.45 (s, 1H, CH).

### Example I26

### Preparation of 3-(5-chloro-thiazole-2-sulfinyl)-propionic acid methyl ester

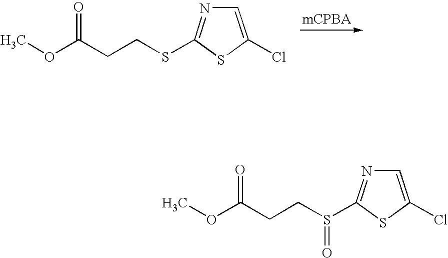

3-(5-Chloro-thiazol-2-ylsulfanyl)-propionic acid methyl ester (20.3 g, 85 mmol) (see Example I25) was dissolved in chloroform (380 ml) and cooled to −10° C. 3-Chloroperoxybenzoic acid (70% by weight) (24.5 g, 100 mmol) was added in portions with cooling. The reaction was stirred for 30 minutes at −10° C. and then concentrated. The residue was partitioned between water and ethyl acetate. The organic extract was washed with aqueous sodium hydrogencarbonate, then with brine, dried over magnesium sulfate and concentrated. The residue was purified by chromatography on silica gel (eluent 1:4 ethyl acetate/dichloromethane) to give the product as a colourless oil (17.7 g, 83% yield).

¹H-NMR (400 MHz, CDCl₃): 2.59-2.67 (m, 1H, 2 CH₂), 2.88-2.96 (m, 1H, ½ CH₂), 3.29-3.37 (m, 1H, 2 CH₂), 3.53-3.60 (m, 1H, ½ CH₂), 3.71 (s, 3H, CH₃), 7.74 (s, 1H, CH).

### Example P14

### Preparation of 5-chloro-2-[5-(2-fluoro-allyloxy)-1-methyl-3-trifluoromethyl-1H-pyrazol-4-ylmethanesulfinyl]-thiazole

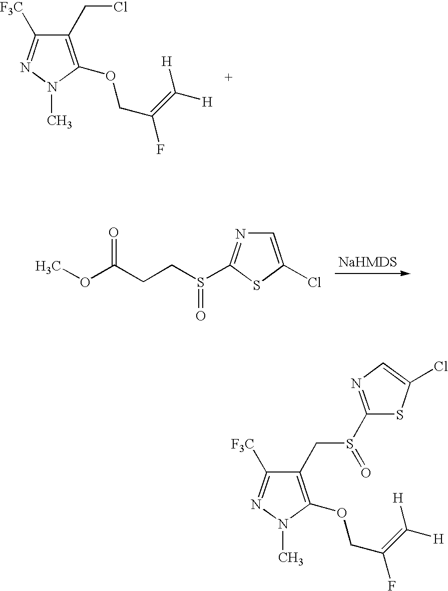

3-(5-Chloro-thiazole-2-sulfinyl)-propionic acid methyl ester (0.78 g, 2.85 mmol) (see Example I26) was dissolved in dry THF (12 ml) and cooled to −78° C. Sodium hexamethyldisilazide (1M in THF) (3.36 ml, 3.36 mmol) was added gradually under nitrogen and the mixture was stirred at −78° C. for 20 minutes. A solution of 4-chloromethyl-5-(2-fluoro-allyloxy)-1-methyl-3-trifluoromethyl-1H-pyrazole (1.0 g, 3.7 mmol) (see Example I7) in THF (2.5 ml) was added gradually and the mixture was stirred at −78° C. for 1 hour. The reaction mixture was allowed to warm to room temperature, and then ethyl acetate (9.5 ml) was added followed by water (9.5 ml). The phases were separated and the aqueous phase extracted three times with ethyl acetate. The combined organic extracts were washed with brine, dried over magnesium sulfate and concentrated. The residue was purified by chromatography on silica gel (eluent 0-30% ethyl acetate in hexane) to give Compound No. 1.124 of Table 32 as a colourless gum which solidified on standing (0.18 g, 16% yield).

### Example P15

### Preparation of 5-chloro-2-[5-(2-methoxy-ethoxy)-1-methyl-3-trifluoromethyl-1H-pyrazol-4-ylmethylsulfanyl]-thiazole

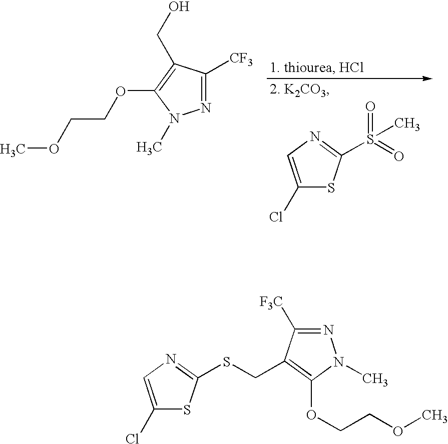

A mixture of [5-(2-methoxy-ethoxy)-1-methyl-3-trifluoromethyl-1H-pyrazol-4-yl]-methanol (1.13 g, 4.45 mmol) (prepared according to Example I2 from 5-(2-methoxy-ethoxy)-1-methyl-3-trifluoromethyl-1H-pyrazole-4-carbaldehyde, which was in turn prepared according to WO 04/014138), thiourea (407 mg, 5.35 mmol), concentrated hydrochloric acid (36% by weight) (1.42 ml), water (5 ml) and 1,4-dioxane (5 ml) was heated in a microwave at 130° C. for 700 seconds. Potassium carbonate (2.78 g, 20 mmol) was added, followed by 5-chloro-2-methanesulfonyl-thiazole (881 mg, 4.45 mmol), water (2 ml) and 1,4-dioxane (2 ml). The reaction mixture was heated for a further 850 seconds in the microwave at 150° C. The reaction was quenched by the addition of water and extracted with ethyl acetate (3×). The combined organic extracts were washed successively with water (2×) and brine, dried over magnesium sulfate and concentrated. The residue was purified by chromatography on silica gel (eluent 20-50% ethyl acetate in hexane) to give Compound No. 1.050 of Table 32 as a pale yellow oil (840 mg, 49% yield).

### Example P16

### Preparation of 5-chloro-2-(5-ethylsulfanyl-1-methyl-3-trifluoromethyl-1H-pyrazole-4-ylmethanesulfonyl)-thiazole

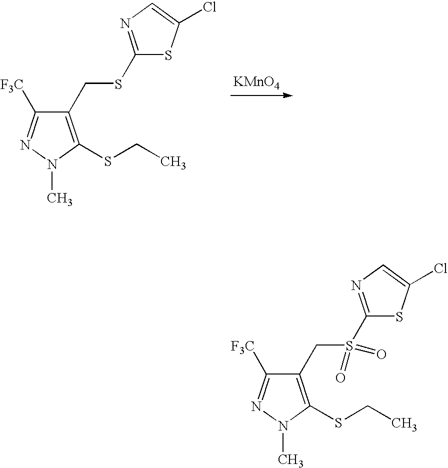

To a solution of 5-chloro-2-(5-ethylsulfanyl-1-methyl-3-trifluoromethyl-1H-pyrazole-4-ylmethanesulfanyl)-thiazole (250 mg, 0.67 mmol) (see Example I15) in acetone (5 ml) was added water (0.2 ml) and potassium permanganate (101 mg, 0.64 mmol). The reaction mixture was stirred at room temperature for 24 hours. The reaction mixture was diluted with water and stirred with aqueous sodium metabisulfite for 10 minutes before being concentrated. The residue was dissolved in water and dichloromethane and the phases separated. The organic phase was dried over sodium sulfate and concentrated. The residue was purified by chromatography over silica gel (eluent 0-50% ethyl acetate in hexane). Compound No. 1.077 of Table 32 was isolated as a white solid (15 mg, 5% yield).

### Example P17

### Preparation of 5-chloro-2-(5-chloro-1-methyl-3-trifluoromethyl-1H-pyrazol-4-ylmethylsulfanyl)-thiazole

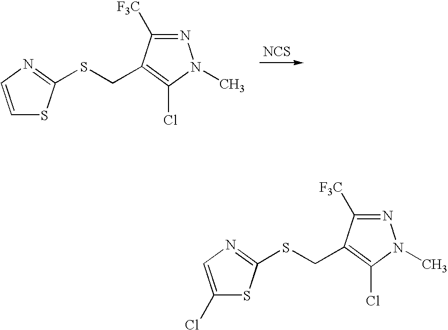

N-chlorosuccinimide (219 mg, 1.65 mmol) was added to a solution of 2-(5-chloro-1-methyl-3-trifluoromethyl-1H-pyrazol-4-ylmethylsulfanyl)-thiazole (Compound 1.020) (500 mg, 1.6 mmol) in dry acetonitrile (10 ml). The reaction mixture was stirred for 16 hours at room temperature and then concentrated. The residue was purified by chromatography on silica gel (eluent 0-50% ethyl acetate in hexane) to yield Compound No. 1.016 of Table 32 as a colourless oil (85% purity) (260 mg, 40% yield).

### Example P18

### Preparation of 2-[1-methyl-5-(2,2,2-trifluoro-ethoxy)-3-trifluoromethyl-1H-pyrazol-4-ylmethylsulfanyl]-thiazole-5-carboxylic acid amide

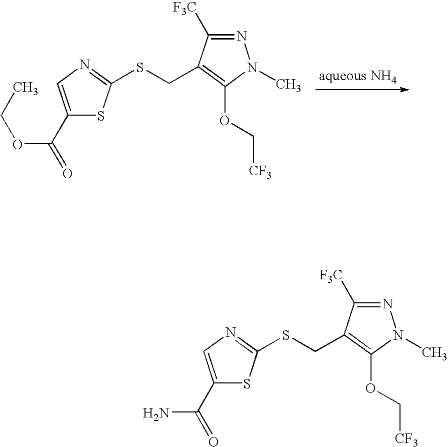

Aqueous ammonia (10 ml) was added to a solution of 2-[1-methyl-5-(2,2,2-trifluoro-ethoxy)-3-trifluoromethyl-1H-pyrazol-4-ylmethylsulfanyl]-thiazole-5-carboxylic acid ethyl ester (Compound No. 1.082 of Table 32) (821 mg, 1.83 mmol) in methanol. The reaction mixture was stirred at room temperature for 60 hours then the pH was adjusted to pH7 by the addition of 20% hydrochloric acid. The mixture was extracted with ethyl acetate (2×) and the combined organic extracts were washed with brine, dried over magnesium sulfate and concentrated to give Compound 1.121 of Table 32 as a white solid (713 mg, 93% yield).

The following compound was also prepared according to this procedure: Compound No. 1.135 of Table 32 using cyclopropylamine as reagent.

### Example P19

### Preparation of 5-bromo-2-[chloro-(5-chloro-1-methyl-3-trifluoromethyl-1H-pyrazol-4-yl)-methanesulfonyl]-thiazole

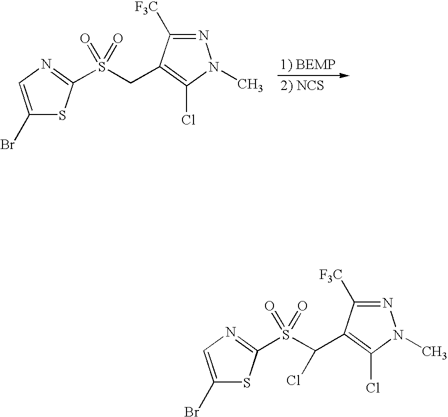

2-tert-Butylimino-2-diethylamino-1,3-dimethyl-perhydro-1,3,2-diazaphosphorine (BEMP) (274.4 mg, 290 μl, 1.0 mmol) was added dropwise over 5 minutes to a solution of 5-bromo-2-(5-chloro-1-methyl-3-trifluoromethyl-1H-pyrazol-4-yl-methanesulfonyl)-thiazole (424 mg, 1.0 mmol) (see Example P3) in dry dichloromethane (10 ml) at −10° C. After stirring for 15 minutes, N-chlorosuccinimide (134 mg, 1.0 mmol) was added in portions over 15 minutes. The reaction mixture was stirred for 1.5 hours. The reaction was quenched by the addition of aqueous hydrochloric acid (2M) and the mixture extracted with dichloromethane (2×50 ml). The combined organic extracts were washed with water, then brine, dried over magnesium sulfate and concentrated. The residue was purified by chromatography on silica gel (eluent 0-50% ethyl acetate in hexane) to give Compound No. 1.030 of Table 32 as a pale cream solid (428 mg, 93% yield).

The following compounds were also prepared according to this procedure: Compound No. 1.034 of Table 32 from Compound No. 1.015 of Table 32, Compound No. 1.039 of Table 32 from Compound No. 1.003 of Table 32 and Compound No. 1.043 of Table 32 from Compound No. 1.018 of Table 32.

### Example P20

### Preparation of 5-bromo-2-[chloro-(5-chloro-1-methyl-3-trifluoromethyl-1H-pyrazol-4-yl)-fluoro-methanesulfonyl]-thiazole

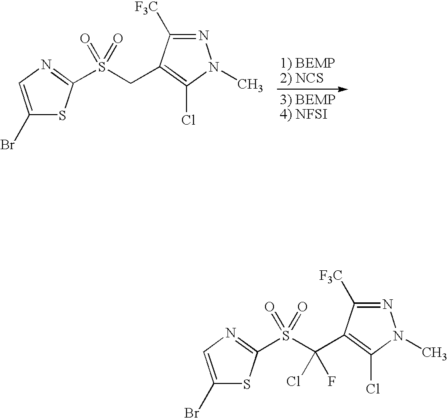

2-tert-butylimino-2-diethylamino-1,3-dimethyl-perhydro-1,3,2-diazaphosphorine (BEMP) (412 mg, 435 μl, 1.5 mmol) was added dropwise over 5 minutes to a solution of 5-bromo-2-(5-chloro-1-methyl-3-trifluoromethyl-1H-pyrazol-4-yl-methanesulfonyl)-thiazole (625 mg, 1.47 mmol) (see Example P3) in dry dichloromethane (20 ml) at 0° C. After stirring for 15 minutes, N-chlorosuccinimide (200 mg, 1.5 mmol) was added in portions over 15 minutes. The reaction mixture was stirred for 1.5 hours. The reaction was quenched by the addition of aqueous hydrochloric acid (2M) and the mixture extracted with dichloromethane (2×50 ml). The combined organic extracts were washed with water, then brine, dried over magnesium sulfate and concentrated. The residue was dissolved in hexane/ethyl acetate (70:30, 2 ml), the solution passed through silica gel and then concentrated to give Compound No. 1.030 of Table 32 as a pale cream solid (500 mg, 74% yield). To a solution of 5-bromo-2-[chloro-(5-chloro-1-methyl-3-trifluoromethyl-1H-pyrazol-4-yl)-methanesulfonyl]-thiazole in dry dichloromethane (15 ml) at 0° C. was added 2-tert-butylimino-2-diethylamino-1,3-dimethyl-perhydro-1,3,2-diazaphosphorine (BEMP) (302 mg, 320 ml, 1.1 mmol) dropwise over 5 minutes. After stirring for 15 minutes, N-fluorobenzenesulfonimide (NFSI) (200 mg, 1.5 mmol) was added in portions over 15 minutes. The reaction mixture was stirred for 1.5 hours. The reaction was quenched by the addition of aqueous hydrochloric acid (2M) and the mixture extracted with dichloromethane (2×50 ml). The combined organic extracts were washed with water, then brine, dried over magnesium sulfate and concentrated. The residue was purified by chromatography on silica gel (eluent 0-40% ethyl acetate in hexane) to give Compound No. 1.031 of Table 32 as a white solid (300 mg, 43% yield).

The following compounds were also prepared according to this procedure: Compound No. 1.035 of Table 32 from Compound No. 1.015 of Table 32, Compound No. 1.040 of Table 32 from Compound No. 1.003 of Table 32 and Compound No. 1.044 of Table 32 from Compound No. 1.018 of Table 32.

### Example P21

### Preparation of 5-bromo-2-[(5-chloro-1-methyl-3-trifluoromethyl-1H-pyrazol-4-yl)-difluoro-methanesulfonyl]-thiazole

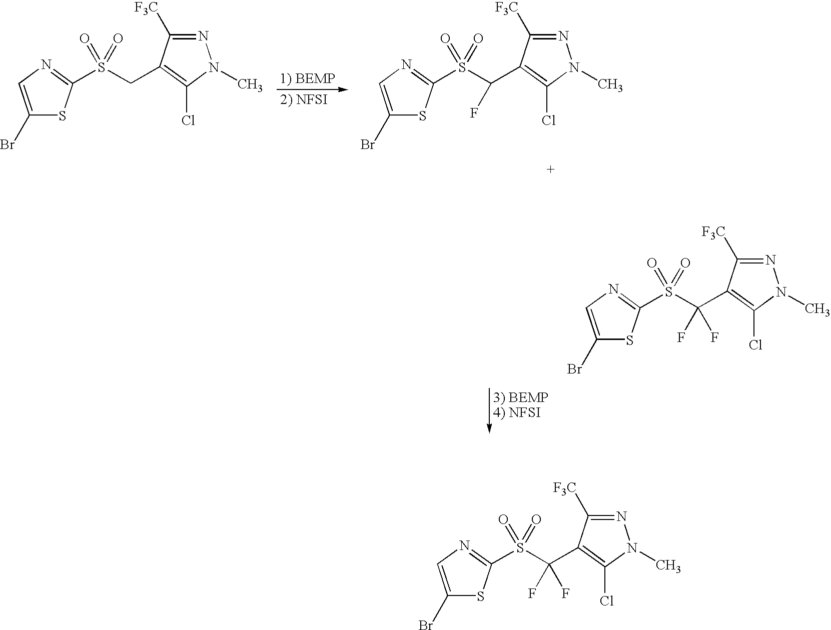

2-tert-butylimino-2-diethylamino-1,3-dimethyl-perhydro-1,3,2-diazaphosphorine (BEMP) (960 mg, 1.01 ml, 3.5 mmol) was added dropwise over 10 minutes to a solution of 5-bromo-2-(5-chloro-1-methyl-3-trifluoromethyl-1H-pyrazol-4-yl-methanesulfonyl)-thiazole (1.5 g, 3.54 mmol) (see Example P3) in dry dichloromethane (20 ml) at 0° C. N-Fluorobenzenesulfonimide (NFSI) (1.1 g, 3.5 mmol) was added in portions over 15 minutes. The reaction mixture was stirred for 1.5 hours. The reaction was quenched by the addition of aqueous hydrochloric acid (2M) and the mixture extracted with dichloromethane (2×25 ml). The combined organic extracts were washed with water, then brine, dried over magnesium sulfate and concentrated. The residue was purified by chromatography on silica gel (eluent 0-40% ethyl acetate in hexane) to give a 3:2 mixture of Compound No. 1.032 of Table 32 and Compound No. 1.033 of Table 32 as a white solid. The mixture was dissolved in dry dichloromethane (20 ml) and cooled to 0° C. To the solution was added 2-tert-butylimino-2-diethylamino-1,3-dimethyl-perhydro-1,3,2-diazaphosphorine (BEMP) (960 mg, 1.01 ml, 3.5 mmol) dropwise over 10 minutes. N-Fluorobenzenesulfonimide (NFSI) (1.1 g, 3.5 mmol) was then added and the reaction mixture was stirred for 2 hours. The reaction was quenched by the addition of aqueous hydrochloric acid (2M) (20 ml) and the mixture extracted with dichloromethane (2×25 ml). The combined organic extracts were washed with water, then brine, dried over magnesium sulfate and concentrated. The residue was purified by chromatography on silica gel (eluent 0-40% ethyl acetate in hexane) to give Compound No. 1.033 as a white solid (440 mg, 27% yield).

The following compounds were also prepared according to this procedure: Compound No. 1.036 of Table 32 and Compound No. 1.037 of Table 32 from Compound No. 1.015 of Table 32, Compound No. 1.041 of Table 32 and Compound No. 1.042 of Table 32 from Compound No. 1.003 of Table 32, and Compound No. 1.045 of Table 32 and Compound No. 1.046 of Table 32 from Compound No. 1.018 of Table 32.

### Example P22

### Preparation of 5-chloro-2-[1-(1-methyl-3-trifluoromethyl-1H-pyrazol-4-yl)-ethanesulfonyl]-thiazole

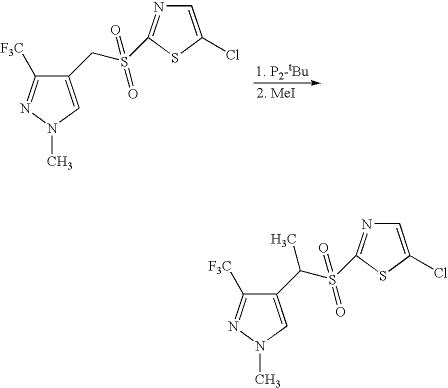

To a solution of 5-chloro-2-(1-methyl-3-trifluoromethyl-1H-pyrazol-4-yl-methanesulfonyl)-thiazole (100 mg, 0.29 mmol) (prepared from 1-methyl-3-trifluoromethyl-1H-pyrazole-4-carboxylic acid, which can be reduced as described in WO 06/240820, brominated as described in Example I3, and derivatised as described in Example P3 or Example P6) in THF (10 ml) was added P₂-^{t}Bu phosphazene base (2M in THF) (144 μl, 0.29 mmol) under nitrogen at room temperature. The mixture was stirred for 10 minutes before adding methyl iodide (36 μl, 0.58 mmol). The mixture was stirred for 30 minutes then quenched with water and extracted with dichloromethane. The organic extract was dried over magnesium sulfate and concentrated. The residue was purified by chromatography on silica gel (eluent 20-50% ethyl acetate in hexane) to give Compound No. 1.108 of Table 32 as a white solid (51 mg, 49% yield).

The following compound was also prepared according to this procedure: Compound No. 1.137 of Table 32 from Compound No. 1.136 of Table 32.

### Example P23

### Preparation of 5-chloro-2-[2-(1-methyl-3-trifluoromethyl-1H-pyrazol-4-yl)-propane-2-sulfonyl]-thiazole

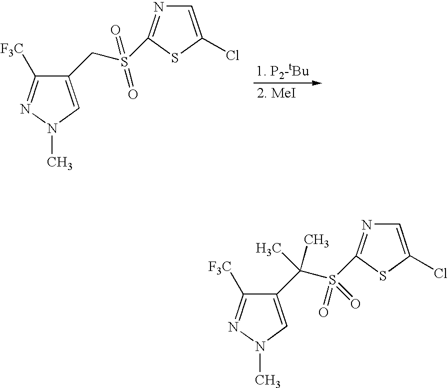

To a solution of 5-chloro-2-(1-methyl-3-trifluoromethyl-1H-pyrazol-4-yl-methanesulfonyl)-thiazole (50 mg, 0.14 mmol) (see Example P9) in THF (10 ml) was added P₂-^{t}Bu phosphazene base (2M in THF) (145 μl, 0.29 mmol) under nitrogen at room temperature. The mixture was stirred for 10 minutes before adding methyl iodide (36 μl, 0.58 mmol). The mixture was stirred for 30 minutes then quenched with water and extracted with dichloromethane. The organic extract was dried over magnesium sulfate and concentrated. The residue was purified by chromatography on silica gel (eluent 20-50% ethyl acetate in hexane) to give Compound No. 1.109 of Table 32 as a white solid (42 mg, 77% yield).

The following compound was also prepared according to this procedure: Compound No. 1.138 of Table 32 from Compound No. 1.136 of Table 32.

### Example P24

### Preparation of 5-chloro-2-[fluoro-(1-methyl-3-trifluoromethyl-1H-pyrazol-4-yl)-methanesulfonyl]-thiazole

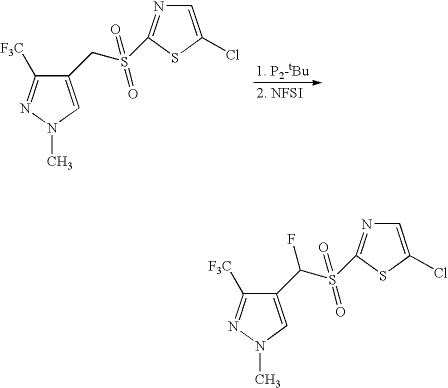

To a solution of 5-chloro-2-(1-methyl-3-trifluoromethyl-1H-pyrazol-4-yl-methanesulfonyl)-thiazole (100 mg, 0.29 mmol) (see Example P9) in THF (5 ml) was added P₂-^{t}Bu phosphazene base (2M in THF) (145 μl, 0.29 mmol) under nitrogen at room temperature. The mixture was stirred for 10 minutes before adding N-fluorobenzenesulfonimide (NFSI) (91 mg, 0.29 mmol). The mixture was stirred for 1 hour then quenched with water and extracted with dichloromethane. The organic extract was dried over magnesium sulfate and concentrated. The residue was purified by chromatography on silica gel (eluent 20-50% ethyl acetate in hexane) to give Compound No. 1.110 of Table 32 as a white solid (54 mg, 51% yield).

### Example P25

### Preparation of 5-chloro-2-[1-fluoro-1-(1-methyl-4-trifluoromethyl-1H-pyrazol-3-yl)-ethanesulfonyl]-thiazole

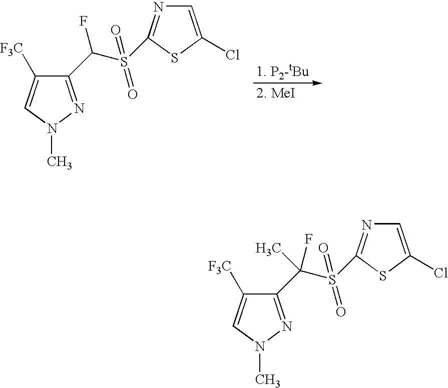

To a solution of 5-chloro-2-[fluoro-(1-methyl-4-trifluoromethyl-1H-pyrazol-3-yl)-methanesulfonyl]-thiazole (54 mg, 0.15 mmol) (see Example P11) in THF (5 ml) was added P₂-^{t}Bu phosphazene base (2M in THF) (74 μl, 0.15 mmol) under nitrogen at room temperature. The mixture was stirred for 10 minutes before adding methyl iodide (2 μl, 0.3 mmol). The mixture was stirred for 30 minutes then quenched with water and extracted with dichloromethane. The organic extract was dried over magnesium sulfate and concentrated. The residue was purified by chromatography on silica gel (eluent 20-50% ethyl acetate in hexane) to give Compound No. 1.111 of Table 32 as a white solid (56 mg, 78% yield).

The compounds mentioned in the following Table can be prepared in analogous manner.

TABLE 32
Novel compounds of formula Ih

| | |
|---|---|
| | (Ih) |
| 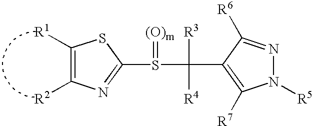 | |

| | | | | | | | | | M.p. | ¹H-NMR (400 |
|---|---|---|---|---|---|---|---|---|---|---|
| No. | R¹ | R² | m | R³ | R⁴ | R⁵ | R⁶ | R⁷ | [° C.] | MHz, CDCl₃) |
| 1.001 | Br | H | 0 | H | H | Me | CF₃ | —OC | oil | 3.75 (s, 3H, |
| | | | | | | | | H₂C | | CH₃), 4.35 (s, |
| | | | | | | | | F₃ | | 2H, CH₂), 4.61 |
| | | | | | | | | | | (m, 2H, CH₂), |
| | | | | | | | | | | 7.6 (s, 1H, |
| | | | | | | | | | | CH). |
| 1.002 | Br | H | 1 | H | H | Me | CF₃ | —OC | 108- | 3.8 (s, 3H, |
| | | | | | | | | H₂C | 109 | CH₃), 4.22 (dd, |
| | | | | | | | | F₃ | | 2H, CH₂), 4.75 |
| | | | | | | | | | | (m, 2H, CH₂), |
| | | | | | | | | | | 7.89 (s, 1H, |
| | | | | | | | | | | CH). |
| 1.003 | Br | H | 2 | H | H | Me | CF₃ | —OC | 85-86 | 3.8 (s, 3H, |
| | | | | | | | | H₂C | | CH₃), 4.55 (s, |
| | | | | | | | | F₃ | | 2H, CH₂), 4.72 |
| | | | | | | | | | | (m, 2H, CH₂), |
| | | | | | | | | | | 7.98 (s, 1H, |
| | | | | | | | | | | CH). |
| 1.004 | Br | H | 0 | H | H | Me | CF₃ | Cl | 58-59 | 3.9 (s, 3H, |
| | | | | | | | | | | CH₃), 4.32 (s, |
| | | | | | | | | | | 2H, CH₂), 7.6 |
| | | | | | | | | | | (s, 1H, CH). |
| 1.005 | Br | H | 1 | H | H | Me | CF₃ | Cl | 128- | 3.9 (s, 3H, |
| | | | | | | | | | 129 | CH₃), 4.3 (dd, |
| | | | | | | | | | | 2H, CH₂), 7.8 |
| | | | | | | | | | | (s, 1H, CH). |
| 1.006 | Br | H | 2 | H | H | Me | CF₃ | Cl | 137-9 | 3.95 (s, 3H, |
| | | | | | | | | | | CH₃), 4.29 (s, |
| | | | | | | | | | | 2H, CH₂), 7.5 |
| | | | | | | | | | | (s, 1H, CH). |
| 1.007 | Cl | H | 0 | H | H | Me | CF₃ | F | oil | 3.78 (s, 3H, |
| | | | | | | | | | | CH₃), 4.29 (s, |
| | | | | | | | | | | 2H, CH₂), 7.5 |
| | | | | | | | | | | (s, 1H, CH). |
| 1.008 | Cl | H | 1 | H | H | Me | CF₃ | F | 86-87 | 3.8 (s, 3H, |
| | | | | | | | | | | CH₃), 4.25 (dd, |
| | | | | | | | | | | 2H, CH₂), 7.5 |
| | | | | | | | | | | (s, 1H, CH). |
| 1.009 | Cl | H | 2 | H | H | Me | CF₃ | F | 77-78 | 3.85 (s, 3H, |
| | | | | | | | | | | CH₃), 4.54 (s, |
| | | | | | | | | | | 2H, CH₂), 7.85 |
| | | | | | | | | | | (s, 1H, CH). |
| 1.010 | Br | H | 0 | H | H | Me | CF₃ | F | oil | 3.79 (s, 3H, |
| | | | | | | | | | | CH₃), 4.29 (dd, |
| | | | | | | | | | | 2H, CH₂), 7.6 |
| | | | | | | | | | | (s, 1H, CH). |
| 1.011 | Br | H | 1 | H | H | Me | CF₃ | F | 94-95 | 3.82 (s, 3H, |
| | | | | | | | | | | CH₃), 4.25 (dd, |
| | | | | | | | | | | 2H, CH₂), 7.83 |
| | | | | | | | | | | (s, 1H, CH). |
| 1.012 | Br | H | 2 | H | H | Me | CF₃ | F | 84-85 | 3.83 (s, 3H, |
| | | | | | | | | | | CH₃), 4.55 (s, |
| | | | | | | | | | | 2H, CH₂), 7.96 |
| | | | | | | | | | | (s, 1H, CH). |
| 1.013 | Cl | H | 0 | H | H | Me | CF₃ | —OC | oil | 3.75 (s, 3H, |
| | | | | | | | | H₂C | | CH₃), 4.35 (s, |
| | | | | | | | | F₃ | | 2H, CH₂), 4.63 |
| | | | | | | | | | | (m, 2H, CH₂), |
| | | | | | | | | | | 7.5 (s, 1H, |
| | | | | | | | | | | CH). |
| 1.014 | Cl | H | 1 | H | H | Me | CF₃ | —OC | 92-93 | 3.81 (s, 3H, |
| | | | | | | | | H₂C | | CH₃), 4.25 (dd, |
| | | | | | | | | F₃ | | 2H, CH₂), 4.8 |
| | | | | | | | | | | (m, 2H, CH₂), |
| | | | | | | | | | | 7.8 (s, 1H, |
| | | | | | | | | | | CH). |
| 1.015 | Cl | H | 2 | H | H | Me | CF₃ | —OC | 94-95 | 3.83 (s, 3H, |
| | | | | | | | | H₂C | | CH₃), 4.55 (s, |
| | | | | | | | | F₃ | | 2H, CH₂), 4.71 |
| | | | | | | | | | | (m, 2H, CH₂), |
| | | | | | | | | | | 7.8 (s, 1H, |
| | | | | | | | | | | CH). |
| 1.016 | Cl | H | 0 | H | H | Me | CF₃ | Cl | oil | 3.87 (s, 3H, |
| | | | | | | | | | | CH₃), 4.31 (s, |
| | | | | | | | | | | 2H, CH₂), 7.52 |
| | | | | | | | | | | (s, 1H, CH). |
| 1.017 | Cl | H | 1 | H | H | Me | CF₃ | Cl | oil | 3.9 (s, 3H, |
| | | | | | | | | | | CH₃), 4.3 (dd, |
| | | | | | | | | | | 2H, CH₂), 7.72 |
| | | | | | | | | | | (s, 1H, CH). |
| 1.018 | Cl | H | 2 | H | H | Me | CF₃ | Cl | 127- | 3.96 (s, 3H, |
| | | | | | | | | | 128 | CH₃), 4.65 (s, |
| | | | | | | | | | | 2H, CH₂), 7.89 |
| | | | | | | | | | | (s, 1H, CH). |
| 1.019 | —(CO) | CF₃ | 2 | H | H | Me | CF₃ | —OC | oil | 1.34 (t, 3H, |
| | OEt | | | | | | | H₂C | | CH₃), 3.77 (s, |
| | | | | | | | | F₃ | | 3H, CH₃), 4.39 |
| | | | | | | | | | | (q, 2H, CH₂), |
| | | | | | | | | | | 4.51 (s,2H, |
| | | | | | | | | | | CH₂), 4.61 (m, |
| | | | | | | | | | | 2H, CH₂). |
| 1.020 | H | H | 0 | H | H | Me | CF₃ | Cl | oil | 3.89 (s, 3H, |
| | | | | | | | | | | CH₃), 4.35 (s, |
| | | | | | | | | | | 2H, CH₂), 7.28 |
| | | | | | | | | | | (s, 1H, CH), |
| | | | | | | | | | | 7.72 (s, 1H, |
| | | | | | | | | | | CH). |
| 1.021 | Br | H | 0 | H | H | Me | CF₃ | —OEt | oil | — |
| 1.022 | Br | H | 1 | H | H | Me | CF₃ | —OEt | 96-97 | 1.43 (t, 3H, |
| | | | | | | | | | | CH₃), 3.75 (s, |
| | | | | | | | | | | 3H, CH₃), 4.15 |
| | | | | | | | | | | (dd, 2H, CH₂), |
| | | | | | | | | | | 4.3 (m, 2H, |
| | | | | | | | | | | CH₂), 7.85 (s, |
| | | | | | | | | | | 1H, CH). |
| 1.023 | Br | H | 2 | H | H | Me | CF₃ | —OEt | 78-79 | 1.45 (t, 3H, |
| | | | | | | | | | | CH₃), 3.75 (s, |
| | | | | | | | | | | 3H, CH₃), 4.3 |
| | | | | | | | | | | (q, 2H, CH₂), |
| | | | | | | | | | | 4.55 (s, 2H, |
| | | | | | | | | | | CH₂), 7.96 (s, |
| | | | | | | | | | | 1H, CH). |
| 1.024 | Br | H | 0 | H | H | Me | CF₃ | —OC | oil | — |
| | | | | | | | | H₂C | | |
| | | | | | | | | HF₂ | | |
| 1.025 | Br | H | 1 | H | H | Me | CF₃ | —OC | 112- | 3.8 (s, 3H, |
| | | | | | | | | H₂C | 113 | CH₃), 4.25 (dd, |
| | | | | | | | | HF₂ | | 2H, CH₂), 4.51 |
| | | | | | | | | | | (m, 2H, CH₂), |
| | | | | | | | | | | 6.1 (tt, 1H, |
| | | | | | | | | | | CH), 7.89 (s, |
| | | | | | | | | | | 1H, CH). |
| 1.026 | Br | H | 2 | H | H | Me | CF₃ | —OC | 102- | 3.8 (s, 3H, |
| | | | | | | | | H₂C | 103 | CH₃), 4.5 (dt, |
| | | | | | | | | HF₂ | | 2H, CH₂), 4.55 |
| | | | | | | | | | | (s, 2H, CH₂), |
| | | | | | | | | | | 6.15 (tt, 1H, |
| | | | | | | | | | | CH), 7.98 (s, |
| | | | | | | | | | | 1H, CH). |
| 1.027 | Br | H | 0 | H | H | Me | CF₃ | —OC | oil | — |
| | | | | | | | | H₂C | | |
| | | | | | | | | F₃ | | |
| 1.028 | H | Br | 1 | H | H | Me | CF₃ | —OC | 113- | 3.8 (s, 3H, |
| | | | | | | | | H₂C | 114 | CH₃), 4.26 (dd, |
| | | | | | | | | F₃ | | 2H, CH₂), 4.7 |
| | | | | | | | | | | (q, 2H, CH₂), |
| | | | | | | | | | | 7.6 (s, 1H, |
| | | | | | | | | | | CH). |
| 1.029 | H | Br | 2 | H | H | Me | CF₃ | —OC | 129- | 3.8 (s, 3H, |
| | | | | | | | | H₂C | 130 | CH₃), 4.6 (s, |
| | | | | | | | | F₃ | | 2H, CH₂), 4.72 |
| | | | | | | | | | | (q, 2H, CH₂), |
| | | | | | | | | | | 7.68 (s, 1H, |
| | | | | | | | | | | CH). |
| 1.030 | Br | H | 2 | Cl | H | Me | CF₃ | Cl | 78- | 3.95 (s, 3H, |
| | | | | | | | | | 79.5 | CH₃), 6.3 (s, |
| | | | | | | | | | | 1H, CH), 8.1 |
| | | | | | | | | | | (s, 1H, CH). |
| 1.031 | Br | H | 2 | Cl | F | Me | CF₃ | Cl | oil | 4.0 (s,3H, |
| | | | | | | | | | | CH₃), 7.9(s, |
| | | | | | | | | | | 1H, CH). |
| 1.032 | Br | H | 2 | F | H | Me | CF₃ | Cl | 167- | 3:2 mixture |
| | | | | | | | | | 170 | with |
| | | | | | | | | | | Compound No. |
| | | | | | | | | | | 1.033 |
| | | | | | | | | | | 3.90 (s, 3H, |
| | | | | | | | | | | CH₃), 6.56 (d, |
| | | | | | | | | | | 1H, CH), 8.05 |
| | | | | | | | | | | (s, 1H, CH). |
| 1.033 | Br | H | 2 | F | F | Me | CF₃ | Cl | 186- | 4.02 (s, 3H, |
| | | | | | | | | | 188 | CH₃), 8.11 (s, |
| | | | | | | | | | | 1H, CH). |
| 1.034 | Cl | H | 2 | Cl | H | Me | CF₃ | —OC | 127.5- | 3.85 (s, 3H, |
| | | | | | | | | H₂C | 129 | CH₃), 4.8 (q, |
| | | | | | | | | F₃ | | 2H, CH₂), 6.28 |
| | | | | | | | | | | (s, 1H, CH), |
| | | | | | | | | | | 8.93 (s, 1H, |
| | | | | | | | | | | CH). |
| 1.035 | Cl | H | 2 | Cl | F | Me | CF₃ | —OC | 109- | 3.89 (s, 3H, |
| | | | | | | | | H₂C | 110 | CH₃), 4.75 |
| | | | | | | | | F₃ | | (dm, 2H, CH₂), |
| | | | | | | | | | | 7.9 (s, 1H, |
| | | | | | | | | | | CH). |
| 1.036 | Cl | H | 2 | F | H | Me | CF₃ | —OC | 135- | 3.85 (s, 3H, |
| | | | | | | | | H₂C | 138 | CH₃), 4.55 (m, |
| | | | | | | | | F₃ | | 1H, ½ CH₂) |
| | | | | | | | | | | 4.85 (m, 1H, ½ |
| | | | | | | | | | | CH₂), 6.55 (d, |
| | | | | | | | | | | 1H, CH), 7.95 |
| | | | | | | | | | | (s, 1H, CH). |
| 1.037 | Cl | H | 2 | F | F | Me | CF₃ | —OC | 115- | 3.85 (s, 3H, |
| | | | | | | | | H₂C | 116 | CH₃), 4.70 (m, |
| | | | | | | | | F₃ | | 2H, CH₂), 8.01 |
| | | | | | | | | | | (s, 1H, CH). |
| 1.038 | —CH═CH— | | 2 | H | H | Me | CF₃ | —OC | — | 3.81 (s, 3H, |
| | CH═CH— | | | | | | | H₂C | | CH₃), 4.71 (s, |
| | | | | | | | | F₃ | | 2H, CH₂), 4.78 |
| | | | | | | | | | | (q, 2H, CH₂), |
| | | | | | | | | | | 7.66 (m, 2H, |
| | | | | | | | | | | 2x CH), 8.01 |
| | | | | | | | | | | (d, 1H, CH), |
| | | | | | | | | | | 8.25 (d, 1H, |
| | | | | | | | | | | CH). |
| 1.039 | Br | H | 2 | Cl | H | Me | CF₃ | —OC | oil | 3.85 (s, 3H, |
| | | | | | | | | H₂C | | CH₃), 4.8 (q, |
| | | | | | | | | F₃ | | 2H, CH₂), 6.28 |
| | | | | | | | | | | (s, 1H, CH), |
| | | | | | | | | | | 8.1 (s, 1H, |
| | | | | | | | | | | CH). |
| 1.040 | Br | H | 2 | Cl | F | Me | CF₃ | —OC | oil | 3.87 (s, 3H, |
| | | | | | | | | H₂C | | CH₃), 4.75 |
| | | | | | | | | F₃ | | (dm, 2H, CH₂), |
| | | | | | | | | | | 8.09 (s, 1H, |
| | | | | | | | | | | CH). |
| 1.041 | Br | H | 2 | F | H | Me | CF₃ | —OC | 145- | 3.82 (s, 3H, |
| | | | | | | | | H₂C | 147 | CH₃), 4.7 (dm, |
| | | | | | | | | F₃ | | 2H, CH₂), 6.55 |
| | | | | | | | | | | (d, 1H, CH), |
| | | | | | | | | | | 8.09 (s, 1H, |
| | | | | | | | | | | CH). |
| 1.042 | Br | H | 2 | F | F | Me | CF₃ | —OC | 119- | 3.86 (s, 3H, |
| | | | | | | | | H₂C | 120 | CH₃), 4.72 (m, |
| | | | | | | | | F₃ | | 2H, CH₂), 8.13 |
| | | | | | | | | | | (s, 1H, CH). |
| 1.043 | Cl | H | 2 | Cl | H | Me | CF₃ | Cl | 99- | 3.98 (s, 3H, |
| | | | | | | | | | 101 | CH₃), 6.26 (s, |
| | | | | | | | | | | 1H, CH), 7.95 |
| | | | | | | | | | | (s, 1H, CH). |
| 1.044 | Cl | H | 2 | Cl | F | Me | CF₃ | Cl | oil | 4.0 (s, 3H, |
| | | | | | | | | | | CH₃), 7.92 (s, |
| | | | | | | | | | | 1H, CH). |
| 1.045 | Cl | H | 2 | F | H | Me | CF₃ | Cl | 133- | 3.97 (s, 3H, |
| | | | | | | | | | 135 | CH₃), 6.55 (d, |
| | | | | | | | | | | 1H, CH), 7.98 |
| | | | | | | | | | | (s, 1H, CH). |
| 1.046 | Cl | H | 2 | F | F | Me | CF₃ | Cl | 136- | 4.0 (s, 3H, |
| | | | | | | | | | 138.5 | CH₃), 8.10 (s, |
| | | | | | | | | | | 1H, CH). |
| 1.047 | H | H | 0 | H | H | Me | CF₃ | —OC | oil | 3.7 (s, 3H, |
| | | | | | | | | H₂C | | CH₃), 4.4 (s, |
| | | | | | | | | F₃ | | 2H, CH₂), |
| | | | | | | | | | | 4.65 (m, 2H, |
| | | | | | | | | | | CH₂), 7.29 (s, |
| | | | | | | | | | | 1H, CH), 7.7 |
| | | | | | | | | | | (s, 1H, CH). |
| 1.048 | H | H | 1 | H | H | Me | CF₃ | —OC | 80-82 | 3.8 (s, 3H, |
| | | | | | | | | H₂C | | CH₃), 4.25 (dd, |
| | | | | | | | | F₃ | | 2H, CH₂), 4.75 |
| | | | | | | | | | | (m, 2H, CH₂), |
| | | | | | | | | | | 7.7 (s, 1H, |
| | | | | | | | | | | CH). 8.05 (s, |
| | | | | | | | | | | 1H, CH). |
| 1.049 | H | H | 2 | H | H | Me | CF₃ | —OC | 87-89 | 3.81 (s, 3H, |
| | | | | | | | | H₂C | | CH₃), 4.6 (s, |
| | | | | | | | | F₃ | | 2H, CH₂), 4.75 |
| | | | | | | | | | | (m, 2H, CH₂), |
| | | | | | | | | | | 7.8 (d, 1H, |
| | | | | | | | | | | CH). 8.1 (s, |
| | | | | | | | | | | 1H, CH). |
| 1.050 | Cl | H | 0 | H | H | Me | CF₃ | —OC | oil | 3.40 (s, 3H, |
| | | | | | | | | H₂C | | CH₃), 3.65 (m, |
| | | | | | | | | H₂O | | 2H, CH₂), 3.75 |
| | | | | | | | | CH₃ | | (s, 3H, CH₃), |
| | | | | | | | | | | 4.33 (m, 2H, |
| | | | | | | | | | | CH₂), 4.38 (s, |
| | | | | | | | | | | 2H, CH₂), 7.5 |
| | | | | | | | | | | (s, 1H, CH). |
| 1.051 | Cl | H | 1 | H | H | Me | CF₃ | —OC | 86-87 | 3.43 (s, 3H, |
| | | | | | | | | H₂C | | CH₃), 3.65 (m, |
| | | | | | | | | H₂O | | 2H, CH₂), 3.80 |
| | | | | | | | | CH₃ | | (s, 3H, CH₃), |
| | | | | | | | | | | 4.28 (m, 2H, |
| | | | | | | | | | | CH₂), 4.37 (m, |
| | | | | | | | | | | 2H, CH₂), 7.75 |
| | | | | | | | | | | (s, 1H, CH). |
| 1.052 | Cl | H | 2 | H | H | Me | CF₃ | —OC | 76-78 | 3.42 (s, 3H, |
| | | | | | | | | H₂C | | CH₃), 3.7 (m, |
| | | | | | | | | H₂O | | 2H, CH₂), 3.8 |
| | | | | | | | | CH₃ | | (s, 3H, CH₃), |
| | | | | | | | | | | 4.38 (m, 2H, |
| | | | | | | | | | | CH₂), 4.6 (s, |
| | | | | | | | | | | 2H, CH₂), 7.87 |
| | | | | | | | | | | (s, 1H, CH). |
| 1.053 | Cl | H | 0 | H | H | Me | —OC | CF₃ | oil | 3.88 (s, 3H, |
| | | | | | | | HF₂ | | | CH₃), 4.24 (q, |
| | | | | | | | | | | 2H, CH₂), 6.86 |
| | | | | | | | | | | (t, 1H, CH), |
| | | | | | | | | | | 7.50 (s, 1H, |
| | | | | | | | | | | CH). |
| 1.054 | Cl | H | 1 | H | H | Me | —OC | CF₃ | gum | 3.91 (s, 3H, |
| | | | | | | | HF₂ | | | CH₃), 4.20 (dq, |
| | | | | | | | | | | 1H, ½ CH₂), |
| | | | | | | | | | | 4.36 (dq, 1H, |
| | | | | | | | | | | ½ CH₂), 6.77 |
| | | | | | | | | | | (t, 1H, CH), |
| | | | | | | | | | | 7.73 (s, 1H, |
| | | | | | | | | | | CH). |
| 1.055 | Cl | H | 2 | H | H | Me | —OC | CF₃ | gum | 3.91 (s, 3H, |
| | | | | | | | HF₂ | | | CH₃), 4.54 (s, |
| | | | | | | | | | | 2H, CH₂), 6.79 |
| | | | | | | | | | | (t, 1H, CH), |
| | | | | | | | | | | 7.88 (s, 1H, |
| | | | | | | | | | | CH). |
| 1.056 | Cl | H | 0 | H | H | Me | CF₃ | —OC | oil | 3.80 (s, 3H, |
| | | | | | | | | HF₂ | | CH₃), 4.31 (s, |
| | | | | | | | | | | 2H, CH₂), 6.68 |
| | | | | | | | | | | (t, 1H, CH), |
| | | | | | | | | | | 7.50 (s, 1H, |
| | | | | | | | | | | CH). |
| 1.057 | Cl | H | 1 | H | H | Me | CF₃ | —OC | 111- | 3.85 (s, 3H, |
| | | | | | | | | HF₂ | 113 | CH₃), 4.25 (dd, |
| | | | | | | | | | | 2H, CH₂), 6.95 |
| | | | | | | | | | | (ds, 1H, CH), |
| | | | | | | | | | | 7.78 (s, 1H, |
| | | | | | | | | | | CH). |
| 1.058 | Cl | H | 2 | H | H | Me | CF₃ | —OC | 118- | 3.89 (s, 3H, |
| | | | | | | | | HF₂ | 119 | CH₃), 4.60 (s, |
| | | | | | | | | | | 2H, CH₂), 6.85 |
| | | | | | | | | | | (t, 1H, CH), |
| | | | | | | | | | | 7.88 (s, 1H, |
| | | | | | | | | | | CH). |
| 1.059 | CN | H | 0 | H | H | Me | CF₃ | —OMe | 141.5- | 3.73 (s, 3H, |
| | | | | | | | | | 142.5 | CH₃), 4.08 (s, |
| | | | | | | | | | | 3H, CH₃), 4.38 |
| | | | | | | | | | | (d, 2H, CH₂), |
| | | | | | | | | | | 7.88 (s, 1H, |
| | | | | | | | | | | CH). |
| 1.060 | CN | H | 0 | H | H | Me | CF₃ | —OC | — | 3.78 (s, 3H, |
| | | | | | | | | H₂C | | CH₃), 4.38 (d, |
| | | | | | | | | F₃ | | 2H, CH₂), 4.62 |
| | | | | | | | | | | (m, 2H, CH₂), |
| | | | | | | | | | | 7.88 (s, 1H, |
| | | | | | | | | | | CH). |
| 1.061 | —NO₂ | H | 0 | H | H | Me | CF₃ | —OC | 87-88 | 3.79 (s, 3H, |
| | | | | | | | | H₂C | | CH₃), 4.60 (m, |
| | | | | | | | | F₃ | | 2H, CH₂), 4.51 |
| | | | | | | | | | | (s, 2H, CH₂), |
| | | | | | | | | | | 8.39 (s, 1H, |
| | | | | | | | | | | CH). |
| 1.062 | —CO | H | 2 | H | H | Me | CF₃ | —OC | 170- | 3.80 (s, 3H, |
| | NH₂ | | | | | | | HF₂ | 171 | CH₃), 4.65 (s, |
| | | | | | | | | | | 2H, CH₂), 7.25 |
| | | | | | | | | | | (t, 1H, CH), |
| | | | | | | | | | | 8.05-8.5 (d, |
| | | | | | | | | | | 2H, NH₂), 8.78 |
| | | | | | | | | | | (s, 1H, CH). |
| 1.063 | —CH═CH— | | 0 | H | H | Me | CF₃ | —OC | 84-85 | 3.78 (s, 3H, |
| | C(Cl)═CH— | | | | | | | H₂C | | CH₃), 4.57 (s, |
| | | | | | | | | F₃ | | 2H, CH₂), 4.7 |
| | | | | | | | | | | (m, 2H, CH₂), |
| | | | | | | | | | | 7.32 (dd, 1H, |
| | | | | | | | | | | CH), 7.68 (d, |
| | | | | | | | | | | 1H, CH), 7.86 |
| | | | | | | | | | | (s, 1H, CH). |
| 1.064 | —CH═CH— | | 1 | H | H | Me | CF₃ | —OC | 188- | 3.80 (s, 3H, |
| | C(Cl)═CH— | | | | | | | H₂C | 190 | CH₃), 4.30 (dd |
| | | | | | | | | F₃ | | 2H, CH₂), 4.80 |
| | | | | | | | | | | (m, 2H, CH₂), |
| | | | | | | | | | | 7.51 (dd, 1H, |
| | | | | | | | | | | CH), 7.95 (d, |
| | | | | | | | | | | 1H, CH), 8.15 |
| | | | | | | | | | | (s, 1H, CH). |
| 1.065 | —CH═CH— | | 2 | H | H | Me | CF₃ | —OC | 136- | 3.83 (s, 3H, |
| | C(Cl)═CH— | | | | | | | H₂C | 137 | CH₃), 4.73 (s, |
| | | | | | | | | F₃ | | 2H, CH₂), 4.77 |
| | | | | | | | | | | (m, 2H, CH₂), |
| | | | | | | | | | | 7.60 (dd, 1H, |
| | | | | | | | | | | CH), 7.95 (d, |
| | | | | | | | | | | 1H, CH), 8.25 |
| | | | | | | | | | | (s, 1H, CH). |
| 1.066 | —CH═C(OEt)— | | 0 | H | H | Me | CF₃ | —OC | 88-89 | 1.45 (t, 3H, |
| | CH═CH— | | | | | | | H₂C | | CH₃), 3.8 (s, |
| | | | | | | | | F₃ | | 3H, CH₃), 4.1 |
| | | | | | | | | | | (q, 2H, CH₂), |
| | | | | | | | | | | 4.55 (s, 2H, |
| | | | | | | | | | | CH₂), 4.7 (q, |
| | | | | | | | | | | 2H, CH₂), 7.05 |
| | | | | | | | | | | (dd, 1H, CH), |
| | | | | | | | | | | 7.25, (d, 1H, |
| | | | | | | | | | | CH), 7.78 (d, |
| | | | | | | | | | | 1H, CH). |
| 1.067 | —CH═C(OEt)— | | 1 | H | H | Me | CF₃ | —OC | 187- | 1.49 (t, 3H, |
| | CH═CH— | | | | | | | H₂C | 189 | CH₃), 3.81 (s, |
| | | | | | | | | F₃ | | 3H, CH₃), 4.15 |
| | | | | | | | | | | (q, 2H, CH₂), |
| | | | | | | | | | | 4.30 (dd, 2H, |
| | | | | | | | | | | CH₂), 4.80 (m, |
| | | | | | | | | | | 2H, CH₂), 7.18 |
| | | | | | | | | | | (dd, 1H, CH), |
| | | | | | | | | | | 7.95 (d, 1H, |
| | | | | | | | | | | CH), 7.95 (d, |
| | | | | | | | | | | 1H, CH). |
| 1.068 | —CH═C(OEt)— | | 2 | H | H | Me | CF₃ | —OC | 183- | 1.50 (t, 3H, |
| | CH═CH— | | | | | | | H₂C | 184 | CH₃), 3.83 (s, |
| | | | | | | | | F₃ | | 3H, CH₃), 4.15 |
| | | | | | | | | | | (q, 2H, CH₂), |
| | | | | | | | | | | 4.66 (s, 2H, |
| | | | | | | | | | | CH₂) 4.76 (q, |
| | | | | | | | | | | 2H, CH₂), 7.25 |
| | | | | | | | | | | (dd, 1H, CH), |
| | | | | | | | | | | 7.36, (d, 1H, |
| | | | | | | | | | | CH), 8.11 (d, |
| | | | | | | | | | | 1H, CH). |
| 1.069 | —CH═C(CH₃)— | | 1 | H | H | Me | CF₃ | —OC | 184- | 2.54, (s, 3H, |
| | CH═CH— | | | | | | | H₂C | 185 | CH₃), 3.80 (s, |
| | | | | | | | | F₃ | | 3H, CH₃), 4.40 |
| | | | | | | | | | | (dd, 2H, CH₂), |
| | | | | | | | | | | 4.80 (m, 2H, |
| | | | | | | | | | | CH₂), 7.42 (d, |
| | | | | | | | | | | 1H, CH), 7.81 |
| | | | | | | | | | | (s, 1H, CH), |
| | | | | | | | | | | 8.15, (d, 1H, |
| | | | | | | | | | | CH). |
| 1.070 | —CH═C(CH₃)— | | 2 | H | H | Me | CF₃ | —OC | 187- | 2.55 (s, 3H, |
| | CH═CH— | | | | | | | H₂C | 189 | CH₃), 3.81 (s, |
| | | | | | | | | F₃ | | 3H, CH₃), 4.70 |
| | | | | | | | | | | (s, 2H, CH₂), |
| | | | | | | | | | | 4.80 (m, 2H, |
| | | | | | | | | | | CH₂), 7.49 (d, |
| | | | | | | | | | | 1H, CH), 7.80 |
| | | | | | | | | | | (s, 1H, CH), |
| | | | | | | | | | | 8.15, (d, 1H, |
| | | | | | | | | | | CH). |
| 1.071 | 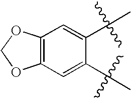 | | 1 | H | H | Me | CF₃ | —OCH₂CF₃ | 177-179 | 3.85 (s, 3H,CH₃), 4.35 (dd,2H, CH₂),4.85, (m, 2H,CH₂), 6.13 (s,2H, CH₂),7.35, (s, 1H,CH), 7.5 (s,1H, CH). |
| 1.072 | 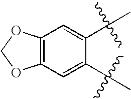 | | 2 | H | H | Me | CF₃ | —OCH₂CF₃ | 142-144 | 3.85 (s, 3H,CH₃), 4.65 (s,2H, CH₂),4.78, (m, 2H,CH₂), 6.19 (s,2H, CH₂),7.32, (s, 1H,CH), 7.56 (s,1H, CH). |
| 1.073 | Me | H | 0 | H | H | Me | CF₃ | —OC | oil | 2.45 (s, 3H, |
| | | | | | | | | H₂C | | CH₃), 3.75 (s, |
| | | | | | | | | F₃ | | 2H, CH₂) 4.35 |
| | | | | | | | | | | (s, 2H, CH₂), |
| | | | | | | | | | | 4.65 (m, 2H, |
| | | | | | | | | | | CH₃), 7.35 (s, |
| | | | | | | | | | | 1H, CH). |
| 1.074 | Me | H | 1 | H | H | Me | CF₃ | —OC | 89-91 | 2.60 (s, 3H, |
| | | | | | | | | H₂C | | CH₃), 3.80 (s, |
| | | | | | | | | F₃ | | 2H, CH₂), 4.20 |
| | | | | | | | | | | (dd, 2H, CH₂), |
| | | | | | | | | | | 4.8 (m, 2H, |
| | | | | | | | | | | CH₃), 7.68 (s, |
| | | | | | | | | | | 1H, CH). |
| 1.075 | Me | H | 2 | H | H | Me | CF₃ | —OC | 96-97 | 2.60 (s, 3H, |
| | | | | | | | | H₂C | | CH₃), 3.81 (s, |
| | | | | | | | | F₃ | | 2H, CH₂), 4.55 |
| | | | | | | | | | | (s, 2H, CH₂), |
| | | | | | | | | | | 4.75 (m, 2H, |
| | | | | | | | | | | CH₃), 7.75 (s, |
| | | | | | | | | | | 1H, CH). |
| 1.076 | Cl | H | 0 | H | H | Me | CF₃ | —SEt | oil | 1.25 (t, 3H, |
| | | | | | | | | | | CH₃), 2.75 (q, |
| | | | | | | | | | | 2H, CH₂), 4.0 |
| | | | | | | | | | | (s, 3H, CH₃), |
| | | | | | | | | | | 4.45 (s, 2H, |
| | | | | | | | | | | CH₂), 7.5 (s, |
| | | | | | | | | | | 1H, CH). |
| 1.077 | Cl | H | 2 | H | H | Me | CF₃ | —SEt | oil | 1.25 (t, 3H, |
| | | | | | | | | | | CH₃), 2.82 (q, |
| | | | | | | | | | | 2H, CH₂), 4.05 |
| | | | | | | | | | | (s, 3H, CH₃), |
| | | | | | | | | | | 4.72 (s, 2H, |
| | | | | | | | | | | CH₂), 7.88 (s, |
| | | | | | | | | | | 1H, CH). |
| 1.078 | Cl | H | 1 | H | H | Me | CF₃ | —(SO) | — | Mixture (1:1) |
| | | | | | | | | Et | | of diastereo- |
| | | | | | | | | | | isomer A: 1.45 |
| | | | | | | | | | | (t, 3H, CH₃), |
| | | | | | | | | | | 3.4 (m, 2H, |
| | | | | | | | | | | CH₂), 4.25 (s, |
| | | | | | | | | | | 3H, CH₃), 4.5 |
| | | | | | | | | | | (dd, 2H, CH₂), |
| | | | | | | | | | | 7.77 (s, 1H, |
| | | | | | | | | | | CH), and dia- |
| | | | | | | | | | | stereoisomer |
| | | | | | | | | | | B: 1.48 (t, 3H, |
| | | | | | | | | | | CH₃), 3.55 (q, |
| | | | | | | | | | | 2H, CH₂), 4.27 |
| | | | | | | | | | | (s, 3H, CH₃), |
| | | | | | | | | | | 4.3 (dd, 2H, |
| | | | | | | | | | | CH₂), 7.85 (s, |
| | | | | | | | | | | 1H, CH). |
| 1.079 | Cl | H | 0 | H | H | Me | CF₃ | —(SO₂) | 93-95 | 1.42 (t, 3H, |
| | | | | | | | | Et | | CH₃), 3.41 (q, |
| | | | | | | | | | | 2H, CH₂), 4.2 |
| | | | | | | | | | | (s, 3H, CH₃), |
| | | | | | | | | | | 4.6 (s, 2H, |
| | | | | | | | | | | CH₂), 7.27 (s, |
| | | | | | | | | | | 1H, CH). |
| 1.080 | Cl | H | 1 | H | H | Me | CF₃ | —(SO₂) | 126- | 1.45 (t, 3H, |
| | | | | | | | | Et | 128 | CH₃), 3.6 (m, |
| | | | | | | | | | | 2H, CH₂), 4.25 |
| | | | | | | | | | | (s, 3H, CH₃), |
| | | | | | | | | | | 4.5 (dd, 2H, |
| | | | | | | | | | | CH₂), 7.77 (s, |
| | | | | | | | | | | 1H, CH). |
| 1.081 | Cl | H | 2 | H | H | Me | CF₃ | —(SO₂) | 145- | 1.5 (t, 3H, |
| | | | | | | | | Et | 148 | CH₃), 3.62 (q, |
| | | | | | | | | | | 2H, CH₂), 4.25 |
| | | | | | | | | | | (s, 3H, CH₃), |
| | | | | | | | | | | 5.08 (s, 2H, |
| | | | | | | | | | | CH₂), 7.88 (s, |
| | | | | | | | | | | 1H, CH). |
| 1.082 | —(CO) | H | 0 | H | H | Me | CF₃ | —OC | oil | 1.35 (t, 3H, |
| | OEt | | | | | | | H₂C | | CH₃), 3.75 (s, |
| | | | | | | | | F₃ | | 3H, CH₃), 4.35 |
| | | | | | | | | | | (q, 2H, CH₂), |
| | | | | | | | | | | 4.49 (s, 2H, |
| | | | | | | | | | | CH₂), 4.65 (m, |
| | | | | | | | | | | 2H, CH₂), 8.21 |
| | | | | | | | | | | (s, 1H, CH). |
| 1.083 | —(CO) | H | 1 | H | H | Me | CF₃ | —OC | oil | 1.40 (t, 3H, |
| | OEt | | | | | | | H₂C | | CH₃), 3.81 (s, |
| | | | | | | | | F₃ | | 3H, CH₃), 4.42 |
| | | | | | | | | | | (q, 2H, CH₂), |
| | | | | | | | | | | 4.31 (dd, 2H, |
| | | | | | | | | | | CH₂), 4.75 (m, |
| | | | | | | | | | | 2H, CH₂), 8.53 |
| | | | | | | | | | | (s, 1H, CH). |
| 1.084 | —(CO) | H | 2 | H | H | Me | CF₃ | —OC | 107- | 1.41 (t, 3H, |
| | OEt | | | | | | | H₂C | 108 | CH₃), 3.81 (s, |
| | | | | | | | | F₃ | | 3H, CH₃), 4.42 |
| | | | | | | | | | | (q, 2H, CH₂), |
| | | | | | | | | | | 4.61 (s, 2H, |
| | | | | | | | | | | CH₂), 4.75 (m, |
| | | | | | | | | | | 2H, CH₂), 8.50 |
| | | | | | | | | | | (s, 1H, CH). |
| 1.085 | —CHF₂ | H | 0 | H | H | Me | CF₃ | —OC | — | 3.77 (s, 3H, |
| | | | | | | | | H₂C | | CH₃), 4.45 (m, |
| | | | | | | | | F₃ | | 2H, CH₂), 4.64 |
| | | | | | | | | | | (q, 2H, CH₂), |
| | | | | | | | | | | 6.84 (t, 1H, |
| | | | | | | | | | | CH), 7.80 (t, |
| | | | | | | | | | | 1H, CH). |
| 1.086 | —CHF₂ | H | 1 | H | H | Me | CF₃ | —OC | oil | 3.82 (s, 3H, |
| | | | | | | | | H₂C | | CH₃), 4.12 (d, |
| | | | | | | | | F₃ | | 1H, ½ CH₂), |
| | | | | | | | | | | 4.39 (d, 1H, ½ |
| | | | | | | | | | | CH₂), 4.73 (m, |
| | | | | | | | | | | 2H, CH₂), 6.96 |
| | | | | | | | | | | (t, 1H, CH), |
| | | | | | | | | | | 8.14 (t, 1H, |
| | | | | | | | | | | CH). |
| 1.087 | —CHF₂ | H | 2 | H | H | Me | CF₃ | —OC | oil | 3.83 (s, 3H, |
| | | | | | | | | H₂C | | CH₃), 4.63 (s, |
| | | | | | | | | F₃ | | 2H, CH₂), 4.72 |
| | | | | | | | | | | (m, 2H, CH₂), |
| | | | | | | | | | | 6.97 (t, 1H, |
| | | | | | | | | | | CH), 8.20 (s, |
| | | | | | | | | | | 1H, CH). |
| 1.088 | Cl | H | 0 | H | H | Me | CF₃ | —SMe | 80-82 | 2.35 (s, 3H, |
| | | | | | | | | | | CH₃), 4.05 (s, |
| | | | | | | | | | | 3H, CH₃), 4.42 |
| | | | | | | | | | | (q, 2H, CH₂), |
| | | | | | | | | | | 7.72 (s, 1H, |
| | | | | | | | | | | CH). |
| 1.089 | Cl | H | 1 | H | H | Me | CF₃ | —SMe | 110- | 3.21 (s, 3H, |
| | | | | | | | | | 112 | CH₃), 4.25 (dd, |
| | | | | | | | | | | 2H, CH₂), 4.3 |
| | | | | | | | | | | (s, 3H, CH₃), |
| | | | | | | | | | | 7.8 (s, 1H, |
| | | | | | | | | | | CH). |
| 1.090 | Cl | H | 1 | H | H | Me | CF₃ | —(SO) | 138- | Diastereoisomer |
| | | | | | | | | Me | 140 | A: 3.32 (s, |
| | | | | | | | | | | 3H, CH₃), 4.3 |
| | | | | | | | | | | (s, 3H, CH₃), |
| | | | | | | | | | | 4.45 (dd, 2H, |
| | | | | | | | | | | CH₂), 7.77 (s, |
| | | | | | | | | | | 1H, CH). |
| 1.091 | Cl | H | 1 | H | H | Me | CF₃ | —(SO) | 149- | Diastereoisomer |
| | | | | | | | | Me | 151 | B: 3.49 (s, |
| | | | | | | | | | | 3H, CH₃), 4.26 |
| | | | | | | | | | | (s, 3H, CH₃), |
| | | | | | | | | | | 4.75 (dd, 2H, |
| | | | | | | | | | | CH₂), 7.78 (s, |
| | | | | | | | | | | 1H, CH). |
| 1.092 | Cl | H | 1 | H | H | Me | CF₃ | —(SO₂) | 160- | 2.3 (s, 3H, |
| | | | | | | | | Me | 161 | CH₃), 4.0 (s, |
| | | | | | | | | | | 3H, CH₃), 4.45 |
| | | | | | | | | | | (s, 2H, CH₂), |
| | | | | | | | | | | 7.5 (s, 1H, |
| | | | | | | | | | | CH). |
| 1.093 | Cl | H | 2 | H | H | Me | CF₃ | —(SO₂) | 190- | 2.3 (s, 3H, |
| | | | | | | | | Me | 193 | CH₃), 4.0 (s, |
| | | | | | | | | | | 3H, CH₃), |
| | | | | | | | | | | 4.45 (q, 2H, |
| | | | | | | | | | | CH₂), 7.5 (s, |
| | | | | | | | | | | 1H, CH). |
| 1.094 | Cl | H | 0 | Me | H | Me | CF₃ | —OC | oil | 1.71 (d, H, |
| | | | | | | | | H₂C | | CH₃), 3.75 (s, |
| | | | | | | | | F₃ | | 3H, CH₃), 4.56 |
| | | | | | | | | | | (dq, 1H, ½ |
| | | | | | | | | | | CH₂), 4.73 (dq, |
| | | | | | | | | | | 1H, ½ CH₂), |
| | | | | | | | | | | 4.91 (q, H, |
| | | | | | | | | | | CH), 7.50 (s, |
| | | | | | | | | | | 1H, CH). |
| 1.095 | Cl | H | 1 | Me | H | Me | CF₃ | —OC | oil | Mixture (3:2) |
| | | | | | | | | H₂C | | of diastereo- |
| | | | | | | | | F₃ | | isomer A: 1.73 |
| | | | | | | | | | | (d, 3H, CH₃), |
| | | | | | | | | | | 3.80 (s, 3H, |
| | | | | | | | | | | CH₃), 4.49 (dq, |
| | | | | | | | | | | 1H, ½ CH₂), |
| | | | | | | | | | | 5.0 (dq, 1H, ½ |
| | | | | | | | | | | CH₂), 4.80 (q, |
| | | | | | | | | | | 1H, CH), 7.75 |
| | | | | | | | | | | (s, 1H, CH); |
| | | | | | | | | | | and diastereo- |
| | | | | | | | | | | isomer B: 1.75 |
| | | | | | | | | | | (d, 3H, CH₃), |
| | | | | | | | | | | 3.83 (s, 3H, |
| | | | | | | | | | | CH₃), 4.45 (dq, |
| | | | | | | | | | | 1H, ½ CH₂), |
| | | | | | | | | | | 4.95 (dq, 1H, |
| | | | | | | | | | | ½ CH₂), 4.73 |
| | | | | | | | | | | (q, 1H, CH), |
| | | | | | | | | | | 7.87 (s, 1H, |
| | | | | | | | | | | CH). |
| 1.096 | Cl | H | 2 | Me | H | Me | CF₃ | —OC | 113- | 1.80 (d, 3H, |
| | | | | | | | | H₂C | 114.5 | CH₃), 3.83 (s, |
| | | | | | | | | F₃ | | 3H, CH₃) 4.49 |
| | | | | | | | | | | (dq, 1H, ½ |
| | | | | | | | | | | CH₂), 5.0 (dq, |
| | | | | | | | | | | 1H, ½ CH₂), |
| | | | | | | | | | | 4.80 (q, 1H, |
| | | | | | | | | | | CH), 7.87 |
| | | | | | | | | | | (s, 1H, CH). |
| 1.097 | —(CO) | H | 0 | H | H | Me | CF₃ | —OC | oil | 2.55 (s, 3H, |
| | Me | | | | | | | H₂C | | CH₃), 3.78 (s, |
| | | | | | | | | F₃ | | 3H, CH₃), 4.4 |
| | | | | | | | | | | (s, 2H, CH₂), |
| | | | | | | | | | | 4.65 (m, 2H, |
| | | | | | | | | | | CH₂), 7.96 (s, |
| | | | | | | | | | | 1H, CH). |
| 1.098 | —(CO) | H | 1 | H | H | Me | CF₃ | —OC | 106- | 2.65 (s, 3H, |
| | Me | | | | | | | H₂C | 108 | CH₃), 3.81 (s, |
| | | | | | | | | F₃ | | 3H,CH₃), |
| | | | | | | | | | | 4.61 (s, 2H, |
| | | | | | | | | | | CH₂), 4.72 (m, |
| | | | | | | | | | | 2H, CH₂), 8.45 |
| | | | | | | | | | | (s, 1H, CH). |
| 1.099 | —(CO) | H | 2 | H | H | Me | CF₃ | —OC | 130- | 2.65 (s, 3H, |
| | Me | | | | | | | H₂C | 132 | CH₃), 3.81 (s, |
| | | | | | | | | F₃ | | 3H, CH₃), 4.31 |
| | | | | | | | | | | (dd, 2H, CH₂), |
| | | | | | | | | | | 4.73 (m, 2H, |
| | | | | | | | | | | CH₂), 8.42 (s, |
| | | | | | | | | | | 1H, CH). |
| 1.100 | —(CO) | H | 0 | H | H | Me | CF₃ | —OC | — | 3.76 (s, 3H, |
| | CF₃ | | | | | | | H₂C | | CH₃), 4.38 (s, |
| | | | | | | | | F₃ | | 2H, CH₂), 4.61 |
| | | | | | | | | | | (m, 2H, CH₂), |
| | | | | | | | | | | 7.89 (s, 1H, |
| | | | | | | | | | | CH). |
| 1.101 | —(CO) | H | 1 | H | H | Me | CF₃ | —OC | gum | 3.80 (s, 3H, |
| | CF₃ | | | | | | | H₂C | | CH₃), 4.17 (m, |
| | | | | | | | | F₃ | | 1H, ½ CH₂), |
| | | | | | | | | | | 4.41 (m, 1H, ½ |
| | | | | | | | | | | CH₂), 4.68 (m, |
| | | | | | | | | | | 2H, CH₂), 8.02 |
| | | | | | | | | | | (d, 1H, CH). |
| 1.102 | Cl | H | 0 | H | H | Me | CF₃ | —OC | oil | 2.21 (dq, 2H, |
| | | | | | | | | H₂C | | CH₂), 3.8 (s, |
| | | | | | | | | H₂C | | 3H, CH₃), 4.25 |
| | | | | | | | | H₂F | | (dd, 2H, CH₂), |
| | | | | | | | | | | 4.35 (m, 2H, |
| | | | | | | | | | | CH₂), 4.7 (m, |
| | | | | | | | | | | 2H, CH₂), 7.75 |
| | | | | | | | | | | (s, 1H, CH). |
| 1.103 | Cl | H | 1 | H | H | Me | CF₃ | —OC | 78-82 | 2.21 (m, 2H, |
| | | | | | | | | H₂C | | CH₂), 3.72 (s, |
| | | | | | | | | H₂C | | 3H, CH₃), 4.32 |
| | | | | | | | | H₂F | | (t, 2H, CH₂), |
| | | | | | | | | | | 4.37 (s, 2H, |
| | | | | | | | | | | CH₂), 4.65 (dt, |
| | | | | | | | | | | 2H, CH₂), 7.75 |
| | | | | | | | | | | (s, 1H, CH). |
| 1.104 | Cl | H | 2 | H | H | Me | CF₃ | —OC | oil | 2.21 (dq, 2H, |
| | | | | | | | | H₂C | | CH₂), 3.8 (s, |
| | | | | | | | | H₂C | | 3H, CH₃), 4.4 |
| | | | | | | | | H₂F | | (t, 2H, CH₂), |
| | | | | | | | | | | 4.57 (s, 2H, |
| | | | | | | | | | | CH₂), 4.7 (dt, |
| | | | | | | | | | | 2H, CH₂), 7.25 |
| | | | | | | | | | | (s, 1H, CH). |
| 1.105 | Cl | H | 0 | H | H | Me | CF₃ | H | oil | 3.89 (s, 3H, |
| | | | | | | | | | | CH₃), 4.34 (s, |
| | | | | | | | | | | 2H, CH₂), 7.44 |
| | | | | | | | | | | (s, 1H, CH), |
| | | | | | | | | | | 7.48 (s, 1H, |
| | | | | | | | | | | CH). |
| 1.106 | Cl | H | 1 | H | H | Me | CF₃ | H | — | 3.97 (s, 3H, |
| | | | | | | | | | | CH₃), 4.20 (d, |
| | | | | | | | | | | 1H, ½ CH₂), |
| | | | | | | | | | | 4.38 (d, 1H, ½ |
| | | | | | | | | | | CH₂), 7.58 (s, |
| | | | | | | | | | | 1H, CH), 7.74 |
| | | | | | | | | | | (s, 1H, CH). |
| 1.107 | Cl | H | 2 | H | H | Me | CF₃ | H | — | 3.97 (s, 3H, |
| | | | | | | | | | | CH₃), 4.61 (s, |
| | | | | | | | | | | 2H, CH₂), 7.68 |
| | | | | | | | | | | (s, 1H, CH), |
| | | | | | | | | | | 7.84 (s, 1H, |
| | | | | | | | | | | CH). |
| 1.108 | Cl | H | 2 | Me | H | Me | CF₃ | H | — | 1.78 (d, 3H, |
| | | | | | | | | | | CH₃), 3.97 (s, |
| | | | | | | | | | | 3H, CH₃), 4.80 |
| | | | | | | | | | | (q, 1H, CH), |
| | | | | | | | | | | 7.75 (s, 1H, |
| | | | | | | | | | | CH), 7.81 (s, |
| | | | | | | | | | | 1H, CH). |
| 1.109 | Cl | H | 2 | Me | Me | Me | CF₃ | H | — | 1.90 (s, 6H, 2x |
| | | | | | | | | | | CH₃), 3.97 (s, |
| | | | | | | | | | | 3H, CH₃), 7.76 |
| | | | | | | | | | | (s, 1H, CH), |
| | | | | | | | | | | 7.81 (s, IH, |
| | | | | | | | | | | CH). |
| 1.110 | Cl | H | 2 | F | H | Me | CF₃ | H | — | 4.04 (s, 3H, |
| | | | | | | | | | | CH₃), 6.57 (d, |
| | | | | | | | | | | 1H, CH), 7.97 |
| | | | | | | | | | | (s, 2H, 2x CH). |
| 1.111 | Cl | H | 2 | F | Me | Me | CF₃ | H | — | 2.17 (t, 3H, |
| | | | | | | | | | | CH₃), 4.00 (s, |
| | | | | | | | | | | 3H, CH₃), 7.76 |
| | | | | | | | | | | (s, 1H, CH), |
| | | | | | | | | | | 7.86 (s, 1H, |
| | | | | | | | | | | CH). |
| 1.112 | Cl | H | 0 | H | H | Me | CF₃ | —OC | 66-68 | 3.77 (s, 3H, |
| | | | | | | | | H(C | | CH₃), 4.36 (s, |
| | | | | | | | | H₂F)₂ | | 2H, CH₂), |
| | | | | | | | | | | 4.66 (m, 2H, |
| | | | | | | | | | | CH₂), 4.85- |
| | | | | | | | | | | 4.74 (m, 3H, |
| | | | | | | | | | | CH, CH₂), |
| | | | | | | | | | | 7.49 (s, 1H, |
| | | | | | | | | | | CH). |
| 1.113 | Cl | H | 1 | H | H | Me | CF₃ | —OC | 75-78 | 3.61 (s, 3H, |
| | | | | | | | | H(C | | CH₃), 4.21 (d, |
| | | | | | | | | H₂F)₂ | | 1H, 1/2 CH₂), |
| | | | | | | | | | | 4.26 (d, 1H, 1/2 |
| | | | | | | | | | | CH₂), 4.65 (dd, |
| | | | | | | | | | | 2H, CH₂), 4.76 |
| | | | | | | | | | | (dd, 2H, CH₂), |
| | | | | | | | | | | 5.12 (m, 1H, |
| | | | | | | | | | | CH), 7.77 (s, |
| | | | | | | | | | | 1H, CH). |
| 1.114 | Cl | H | 2 | H | H | Me | CF₃ | —OC | 99- | 3.61 (s, 3H, |
| | | | | | | | | H(C | 102 | CH₃), 4.60 (s, |
| | | | | | | | | H₂F)₂ | | 2H, CH₂), 4.68 |
| | | | | | | | | | | (s, 2H, CH₂), |
| | | | | | | | | | | 4.80 (s, 2H, |
| | | | | | | | | | | CH₂), 5.01 (s, |
| | | | | | | | | | | 1H, CH), 7.88 |
| | | | | | | | | | | (s, 1H, CH). |
| 1.115 | Cl | H | 0 | H | H | Me | CF₃ | —OC | oil | 3.7 (s, 3H, |
| | | | | | | | | H₂C | | CH₃), 4.36 (s, |
| | | | | | | | | HF₂ | | 2H, CH₂), 4.43 |
| | | | | | | | | | | (dt, 2H, CH₂), |
| | | | | | | | | | | 6.07 (tt, 1H, |
| | | | | | | | | | | CH), 7.49 (s, |
| | | | | | | | | | | 1H, CH). |
| 1.116 | Cl | H | 1 | H | H | Me | CF₃ | —OC | 86-88 | 3.81 (s, 3H, |
| | | | | | | | | H₂C | | CH₃), 4.14 (d, |
| | | | | | | | | HF₂ | | 1H, ½ CH₂), |
| | | | | | | | | | | 4.30 (d, 1H, ½ |
| | | | | | | | | | | CH₂), 4.51 (m, |
| | | | | | | | | | | 2H, CH₂), 6.08 |
| | | | | | | | | | | (tt, 1H, CH), |
| | | | | | | | | | | 7.78 (s, 1H, |
| | | | | | | | | | | CH). |
| 1.117 | Cl | H | 2 | H | H | Me | CF₃ | —OC | 74.5- | 3.83 (s, 3H, |
| | | | | | | | | H₂C | 77 | CH₃), 4.52 (dt, |
| | | | | | | | | HF₂ | | 2H, CH₂), 4.57 |
| | | | | | | | | | | (s, 2H, CH₂), |
| | | | | | | | | | | 6.14 (tt, 1H, |
| | | | | | | | | | | CH), 7.88 (s, |
| | | | | | | | | | | 1H, CH). |
| 1.118 | Cl | H | 0 | H | H | Me | CF₃ | —OC | oil | 3.76 (s, 3H, |
| | | | | | | | | H₂C | | CH₃), 4.36 (s, |
| | | | | | | | | F₂C | | 2H, CH₂), 4.64 |
| | | | | | | | | HF₂ | | (tt, 2H, CH₂), |
| | | | | | | | | | | 6.00 (tt, 1H, |
| | | | | | | | | | | CH), 7.49 (s, |
| | | | | | | | | | | 1H, CH). |
| 1.119 | Cl | H | 1 | H | H | Me | CF₃ | —OC | 67-69 | 3.81 (s, 3H, |
| | | | | | | | | H₂C | | CH₃), 4.12 (d, |
| | | | | | | | | F₂C | | 1H, ½ CH₂), |
| | | | | | | | | HF₂ | | 4.29 (d, 1H, ½ |
| | | | | | | | | | | CH₂), 4.81 (q, |
| | | | | | | | | | | 2H, CH₂), 6.01 |
| | | | | | | | | | | (tt, 1H, CH), |
| | | | | | | | | | | 7.78 (s, 1H, |
| | | | | | | | | | | CH). |
| 1.120 | Cl | H | 2 | H | H | Me | CF₃ | —OC | oil | 3.83 (s, 3H, |
| | | | | | | | | H₂C | | CH₃), 4.57 (s, |
| | | | | | | | | F₂C | | 2H, CH₂), 4.72 |
| | | | | | | | | HF₂ | | (t, 2H, CH₂), |
| | | | | | | | | | | 6.04 (tt, 1H, |
| | | | | | | | | | | CH), 7.88 (s, |
| | | | | | | | | | | 1H, CH). |
| 1.121 | —CO | H | 0 | H | H | Me | CF₃ | —OC | 118- | 3.75 (s, 3H, |
| | NH₂ | | | | | | | H₂C | 120 | CH₃), 4.45 (s, |
| | | | | | | | | F₃ | | 2H, CH₂), 4.65 |
| | | | | | | | | | | (m, 2H, CH₂), |
| | | | | | | | | | | 5.90 (bs, 2H, |
| | | | | | | | | | | NH₂), 8.00 |
| | | | | | | | | | | (s, 1H, CH). |
| 1.122 | —CO | H | 1 | H | H | Me | CF₃ | —OC | 110- | 3.80 (s, 3H, |
| | NH₂ | | | | | | | H₂C | 113 | CH₃), 4.35 (dd, |
| | | | | | | | | F₃ | | 2H, CH₂), 4.75 |
| | | | | | | | | | | (m, 2H, CH₂), |
| | | | | | | | | | | 5.90 (bs, 2H, |
| | | | | | | | | | | NH₂), 8.30 (s, |
| | | | | | | | | | | 1H, CH). |
| 1.123 | —CO | H | 2 | H | H | Me | CF₃ | —OC | 150- | 3.81 (s, 3H, |
| | NH₂ | | | | | | | H₂C | 153 | CH₃), 4.62 (s, |
| | | | | | | | | F₃ | | 2H, CH₂), 4.72 |
| | | | | | | | | | | (m, 2H, CH₂), |
| | | | | | | | | | | 5.90 (bs, 2H, |
| | | | | | | | | | | NH₂), 8.31 (s, |
| | | | | | | | | | | 1H, CH). |
| 1.124 | Cl | H | 1 | H | H | Me | CF₃ | —OC | waxy | 3.79 (s, 3H, |
| | | | | | | | | H₂C | solid | CH₃), 4.22 (d, |
| | | | | | | | | F═CH₂ | | 2H, CH₂), 4.79 |
| | | | | | | | | | | (dd, 1H, ½ |
| | | | | | | | | | | CH₂), 4.81 (dd, |
| | | | | | | | | | | 2H, CH₂), 4.91 |
| | | | | | | | | | | (dd, 1H, ½ |
| | | | | | | | | | | CH₂), 7.78 (s, |
| | | | | | | | | | | 1H, CH). |
| 1.125 | Cl | H | 2 | H | H | Me | CF₃ | —OC | gum | 3.80 (s, 3H, |
| | | | | | | | | H₂C | | CH₃), 4.58 (s, |
| | | | | | | | | F═CH₂ | | 2H, CH₂), 4.81 |
| | | | | | | | | | | (dd, 1H, ½ |
| | | | | | | | | | | CH₂), 4.82 (d, |
| | | | | | | | | | | 2H, CH₂), 4.97 |
| | | | | | | | | | | (dd, 1H, ½ |
| | | | | | | | | | | CH₂), 7.88 (s, |
| | | | | | | | | | | 1H, CH). |
| 1.126 | Cl | H | 0 | H | H | Me | CF₃ | —OC | oil | 1.6 (d, 3H, |
| | | | | | | | | (CH₃) | | CH₃), 3.74 (s, |
| | | | | | | | | HCF₃ | | 3H, CH₃), 4.33 |
| | | | | | | | | | | (d, 1H, ½ |
| | | | | | | | | | | CH₂), 4.37 (d, |
| | | | | | | | | | | 1H, ½ CH₂), |
| | | | | | | | | | | 4.84 (m, 1H, |
| | | | | | | | | | | CH), 7.5 (s, |
| | | | | | | | | | | 1H, CH). |
| 1.127 | Cl | H | 1 | H | H | Me | CF₃ | —OC | — | Mixture (1:1) |
| | | | | | | | | (CH₃) | | of diastereo- |
| | | | | | | | | HCF₃ | | isomer A: 1.55 |
| | | | | | | | | | | (d, 3H, CH₃), |
| | | | | | | | | | | 3.8 (s, 3H, |
| | | | | | | | | | | CH₃), 4.03 (d, |
| | | | | | | | | | | 1H, ½ CH₂), |
| | | | | | | | | | | 4.30 (d, 1H, ½ |
| | | | | | | | | | | CH₂), 5.18 (m, |
| | | | | | | | | | | 1H, CH), 7.78 |
| | | | | | | | | | | (s, 1H, CH); |
| | | | | | | | | | | and diastereo- |
| | | | | | | | | | | isomer B: 1.6 |
| | | | | | | | | | | (d, 3H, CH₃), |
| | | | | | | | | | | 3.9 (s, 3H, |
| | | | | | | | | | | CH₃), 4.12 (d, |
| | | | | | | | | | | 1H, ½ CH₂), |
| | | | | | | | | | | 4.30 (d, 1H, ½ |
| | | | | | | | | | | CH₂), 5.18 (m, |
| | | | | | | | | | | 1H, CH), 7.79 |
| | | | | | | | | | | (s, 1H, CH). |
| 1.128 | Cl | H | 1 | H | H | Me | CF₃ | —OC | — | Diastereoisomer |
| | | | | | | | | (CH₃) | | A: 1.55 (d, |
| | | | | | | | | HCF₃ | | 3H, CH₃), 3.8 |
| | | | | | | | | | | (s, 3H, CH₃), |
| | | | | | | | | | | 4.03 (d, 1H, ½ |
| | | | | | | | | | | CH₂), 4.30 (d, |
| | | | | | | | | | | 1H, ½ CH₂), |
| | | | | | | | | | | 5.18 (m, 1H, |
| | | | | | | | | | | CH), 7.78 (s, |
| | | | | | | | | | | 1H, CH). |
| 1.129 | Cl | H | 1 | H | H | Me | CF₃ | —OC | — | Diastereoisomer |
| | | | | | | | | (CH₃) | | B: 1.6 (d, |
| | | | | | | | | HCF₃ | | 3H, CH₃), 3.9 |
| | | | | | | | | | | (s, 3H, CH₃), |
| | | | | | | | | | | 4.12 (d, 1H, ½ |
| | | | | | | | | | | CH₂), 4.30 (d, |
| | | | | | | | | | | 1H, ½ CH₂), |
| | | | | | | | | | | 5.18 (m, 1H, |
| | | | | | | | | | | CH), 7.79 (s, |
| | | | | | | | | | | 1H, CH). |
| 1.130 | Cl | H | 2 | H | H | Me | CF₃ | —OC | 99 | 1.62 (d, 3H, |
| | | | | | | | | (CH₃) | | CH₃), 3.8 (s, |
| | | | | | | | | HCF₃ | | 3H, CH₃), 4.57 |
| | | | | | | | | | | (s, 2H, CH₂), |
| | | | | | | | | | | 5.0 (m, 1H, |
| | | | | | | | | | | CH), 7.88 (s, |
| | | | | | | | | | | 1H, CH). |
| 1.131 | Cl | H | 0 | H | H | Me | CF₃ | —OC | gum | 1.40 (d, 3H, |
| | | | | | | | | (CH₃) | | CH₃), 3.75 (s, |
| | | | | | | | | HCF₃ | | 3H, CH₃), 4.35 |
| | | | | | | | | | | (s, 2H, CH₂), |
| | | | | | | | | | | 4.43 (m, 1H, ½ |
| | | | | | | | | | | CH₂), 4.55 (m, |
| | | | | | | | | | | 1H, ½ CH₂), |
| | | | | | | | | | | 4.65 (m, 1H, |
| | | | | | | | | | | CH), 7.49 (s, |
| | | | | | | | | | | 1H, CH). |
| 1.132 | Cl | H | 1 | H | H | Me | CF₃ | —OC | — | Mixture (1:1) |
| | | | | | | | | (CH₃) | | of diastereo- |
| | | | | | | | | HCF₃ | | isomer A: 1.37 |
| | | | | | | | | | | (d, 3H, CH₃), |
| | | | | | | | | | | 3.79 (s, 3H, |
| | | | | | | | | | | CH₃), 4.1-4.3 |
| | | | | | | | | | | (m, 2H, CH₂), |
| | | | | | | | | | | 4.4-4.65 (m, |
| | | | | | | | | | | 2H, CH₂), 4.7- |
| | | | | | | | | | | 4.9 (m, 1H, |
| | | | | | | | | | | CH), 7.76 (s, |
| | | | | | | | | | | 1H, CH); and |
| | | | | | | | | | | diastereoisomer |
| | | | | | | | | | | B: 1.43 (d, |
| | | | | | | | | | | 3H, CH₃), 3.79 |
| | | | | | | | | | | (s, 3H, CH₃), |
| | | | | | | | | | | 4.1-4.3 (m, 2H, |
| | | | | | | | | | | CH₂), 4.4-4.65 |
| | | | | | | | | | | (m, 2H, CH₂), |
| | | | | | | | | | | 4.7-4.9 (m, 1H, |
| | | | | | | | | | | CH), 7.76 (s, |
| | | | | | | | | | | 1H, CH). |
| 1.133 | Cl | H | 2 | H | H | Me | CF₃ | —OC | 90 | 1.41 (d, 3H, |
| | | | | | | | | (CH₃) | | CH₃), 3.79 (s, |
| | | | | | | | | HCH₂F | | 3H, CH₃), 4.47 |
| | | | | | | | | | | (m, 1H, ½ |
| | | | | | | | | | | CH₂), 4.54 (m, |
| | | | | | | | | | | 1H, ½CH₂), |
| | | | | | | | | | | 4.59 (s, 2H, |
| | | | | | | | | | | CH₂), 4.74 (m, |
| | | | | | | | | | | 1H, CH), 7.89 |
| | | | | | | | | | | (s, 1H, CH). |
| 1.134 | Cl | H | 0 | H | H | Me | —OC | —CF₃ | gum | 3.82 (d, 3H, |
| | | | | | | | H₂C | | | CH₃), 4.23 (d, |
| | | | | | | | F₃ | | | 2H, CH₂), 4.55 |
| | | | | | | | | | | (q, 2H, CH₂), |
| | | | | | | | | | | 7.49 (s, 1H, |
| | | | | | | | | | | CH). |
| 1.135 | Cl | H | 1 | H | H | Me | —OC | —CF₃ | — | 3.86 (s, 3H, |
| | | | | | | | H₂C | | | CH₃), 4.21 (d, |
| | | | | | | | F₃ | | | 1H, ½ CH₂), |
| | | | | | | | | | | 4.32 (d, 1H, ½ |
| | | | | | | | | | | CH₂), 4.35- |
| | | | | | | | | | | 4.50 (m, 2H, |
| | | | | | | | | | | CH₂), 7.69 (s, |
| | | | | | | | | | | 1H, CH). |
| 1.136 | Cl | H | 2 | H | H | Me | —OC | —CF₃ | — | 3.86 (d, 3H, |
| | | | | | | | H₂C | | | CH₃), 4.44 (q, |
| | | | | | | | F₃ | | | 2H, CH₂), 4.54 |
| | | | | | | | | | | (d, 2H, CH₂), |
| | | | | | | | | | | 7.85 (s, 1H, |
| | | | | | | | | | | CH). |
| 1.137 | Cl | H | 2 | Me | H | Me | —OC | —CF₃ | — | 1.85 (d, 3H, |
| | | | | | | | H₂C | | | CH₃), 3.85 (s, |
| | | | | | | | F₃ | | | 3H, CH₃), |
| | | | | | | | | | | 4.35-4.45 (m, |
| | | | | | | | | | | 1H, ½ CH₂), |
| | | | | | | | | | | 4.54-4.63 (m, |
| | | | | | | | | | | 1H, ½ CH₂), |
| | | | | | | | | | | 4.78 (q, 1H, |
| | | | | | | | | | | CH), 7.85 (s, |
| | | | | | | | | | | 1H, CH). |
| 1.138 | Cl | H | 2 | Me | Me | Me | —OC | —CF₃ | gum | 1.96 (s, 6H, 2x |
| | | | | | | | H₂C | | | CH₃), 3.92 (q, |
| | | | | | | | F₃ | | | 3H, CH₃), 4.41 |
| | | | | | | | | | | (q, 2H, CH₂), |
| | | | | | | | | | | 7.81 (s, 1H, |
| | | | | | | | | | | CH). |
| 1.139 | —CO | H | 0 | H | H | Me | CF₃ | —OC | 199- | 0.69-0.90 (m, |
| | NH^{c} | | | | | | | H₂C | 201 | 4H, 2x CH₂), |
| | Pr | | | | | | | F₃ | | 2.89 (m, 1H, |
| | | | | | | | | | | CH), 3.79 (s, |
| | | | | | | | | | | 3H, CH₃), 4.43 |
| | | | | | | | | | | (s, 2H, CH₂), |
| | | | | | | | | | | 4.63 (m, 2H, |
| | | | | | | | | | | CH₂), 6.00 (bs, |
| | | | | | | | | | | 1H, NH), 7.93 |
| | | | | | | | | | | (s, 1H, CH). |
| 1.140 | —CO | H | 1 | H | H | Me | CF₃ | —OC | 146- | 0.75-.90 (m, |
| | NH^{c} | | | | | | | H₂C | 148 | 4H, 2x CH₂), |
| | Pr | | | | | | | F₃ | | 2.91 (m, 1H, |
| | | | | | | | | | | CH), 3.80 (s, |
| | | | | | | | | | | 3H, CH₃), 4.10 |
| | | | | | | | | | | (d, 1H, 1/2 |
| | | | | | | | | | | CH₂), 4.32 (d, |
| | | | | | | | | | | 1H, 1/2 CH₂), |
| | | | | | | | | | | 4.73 (m, 2H, |
| | | | | | | | | | | CH₂), 6.20 (bs, |
| | | | | | | | | | | 1H, NH), 8.24 |
| | | | | | | | | | | (s, 1H, CH). |
| 1.141 | —CO | H | 2 | H | H | Me | CF₃ | —OC | 215- | 0.70-0.90 (m, |
| | NH^{c} | | | | | | | H₂C | 217 | 4H, 2x CH₂), |
| | Pr | | | | | | | F₃ | | 2.90 (m, 1H, |
| | | | | | | | | | | CH), 3.86 (s, |
| | | | | | | | | | | 3H, CH₃), 4.60 |
| | | | | | | | | | | (s, 2H, CH₂), |
| | | | | | | | | | | 4.73 (m, 2H, |
| | | | | | | | | | | CH₂), 6.21 (bs, |
| | | | | | | | | | | 1H, NH), 8.25 |
| | | | | | | | | | | (s, 1H, CH). |
| 1.142 | —CO | H | 0 | H | H | Me | CF₃ | —OC | — | 1.45 (s, 9H, |
| | NH^{t} | | | | | | | H₂C | | ^{t}Bu), 3.76 (3H, |
| | Bu | | | | | | | F₃ | | s, CH₃), 4.44 |
| | | | | | | | | | | (s, 2H, CH₂), |
| | | | | | | | | | | 4.64 (m, 2H, |
| | | | | | | | | | | CH₂), 5.62 (bs, |
| | | | | | | | | | | 1H, NH), 7.88 |
| | | | | | | | | | | (s, 1H, CH). |
| 1.143 | —CO | H | 1 | H | H | Me | CF₃ | —OC | — | 1.48 (s, 9H, |
| | NH^{t} | | | | | | | H₂C | | ^{t}Bu), 3.81 (3H, |
| | Bu | | | | | | | F₃ | | s, CH₃), 4.09 |
| | | | | | | | | | | (d, 1H, ½ |
| | | | | | | | | | | CH₂), 4.36 (d, |
| | | | | | | | | | | 1H, ½ CH₂) |
| | | | | | | | | | | 4.75 (m, 2H, |
| | | | | | | | | | | CH₂), 5.82 (bs, |
| | | | | | | | | | | 1H, NH), 8.19 |
| | | | | | | | | | | (s, 1H, CH). |
| 1.144 | —CO | H | 2 | H | H | Me | CF₃ | —OC | — | 1.48 (s, 9H, |
| | NH^{t} | | | | | | | H₂C | | ^{t}Bu), 3.83 (3H, |
| | Bu | | | | | | | F₃ | | s, CH₃), 4.60 |
| | | | | | | | | | | (s, 2H, CH₂), |
| | | | | | | | | | | 4.72 (m, 2H, |
| | | | | | | | | | | CH₂), 5.58 (bs, |
| | | | | | | | | | | 1H, NH), 8.20 |
| | | | | | | | | | | (s, 1H, CH). |
| 1.145 | —SiMe₃ | H | 0 | H | H | Me | CF₃ | —OC | — | 0.3 (s, 9H, 3x |
| | | | | | | | | H₂C | | CH₃), 3.75 (s, |
| | | | | | | | | F₃ | | 3H, CH₃), 4.4 |
| | | | | | | | | | | (s, 2H, CH₂), |
| | | | | | | | | | | 4.63 (m, 2H, |
| | | | | | | | | | | CH₂), 7.65 (s, |
| | | | | | | | | | | 1H, CH). |
| 1.146 | —SiMe₃ | H | 1 | H | H | Me | CF₃ | —OC | — | 0.4 (s, 9H, 3x |
| | | | | | | | | H₂C | | CH₃), 3.8 (s, |
| | | | | | | | | F₃ | | 3H, CH₃), 4.25 |
| | | | | | | | | | | (s, 2H, CH₂), |
| | | | | | | | | | | 4.78 (m, 2H, |
| | | | | | | | | | | CH₂), 7.96 (s, |
| | | | | | | | | | | 1H, CH). |
| 1.147 | —SiMe₃ | H | 2 | H | H | Me | CF₃ | —OC | — | 0.41 (s, 9H, 3x |
| | | | | | | | | H₂C | | CH₃), 3.81 (s, |
| | | | | | | | | F₃ | | 3H, CH₃), 4.59 |
| | | | | | | | | | | (s, 2H, CH₂), |
| | | | | | | | | | | 4.75 (m, 2H, |
| | | | | | | | | | | CH₂), 8.2 (s, |
| | | | | | | | | | | 1H, CH). |

Abbreviations used in the Table: bs = broad singlet, s = singlet, d = doublet, dd = doublet of doublet, dt = doublet of triplet, dq = doublet of quartet, dm = doublet of multiplet, t = triplet, tt = triplet of triplet, m = multiplet, q = quartet.

### BIOLOGICAL EXAMPLES

### Example B1

### Herbicidal Action Prior to Emergence of the Plants (Pre-Emergence Action)

Monocotyledonous and dicotyledonous test plants were sown in seed trays in standard compost. The trays were watered twice daily or as required. The chemicals were applied by track sprayer at the soil surface. The application was carried out with an aqueous suspension of the test substances, prepared as a formulation of 50% acetone in water with 0.5% Tween 20™ (CAS RN 9005-64-5), to achieve a field equivalent of 1000 l/ha. The application rate of the test substances was 500 g/ha. A visual assessment of the herbicidal effect was made at 13 days after application. The following percentage scale was used for assessment: 0, 5, 10, 20, 30, 40, 50, 60, 70, 80, 90, 95, 100 (where 0 is no damage to plants and 100 is plants are completely dead).

| Compound | Rate | | | | |
|---|---|---|---|---|---|
| No. | (g/ha) | ECHCG | ALOMY | AMARE | STEME |
| 1.001 | 500 | 95 | 90 | 90 | 60 |
| 1.002 | 500 | 95 | 95 | 100 | 100 |
| 1.003 | 500 | 95 | 95 | 95 | 70 |
| 1.004 | 500 | 95 | 95 | 95 | 60 |
| 1.005 | 500 | 95 | 95 | 100 | 60 |
| 1.006 | 500 | 95 | 90 | 95 | 30 |
| 1.007 | 500 | 95 | 95 | 80 | 40 |
| 1.008 | 500 | 95 | 95 | 100 | 80 |
| 1.009 | 500 | 95 | 0 | 95 | 0 |
| 1.010 | 500 | 80 | 60 | 70 | 0 |
| 1.011 | 500 | 95 | 95 | 95 | 10 |
| 1.012 | 500 | 95 | 70 | 90 | 0 |
| 1.013 | 500 | 80 | 95 | 95 | 95 |
| 1.014 | 500 | 95 | 100 | 100 | 95 |
| 1.015 | 500 | 95 | 90 | 95 | 80 |
| 1.017 | 500 | 95 | 95 | 95 | 95 |
| 1.018 | 500 | 95 | 60 | 100 | 0 |
| 1.022 | 500 | 95 | 95 | 100 | 90 |
| 1.023 | 500 | 90 | 50 | 100 | 30 |
| 1.025 | 500 | 95 | 95 | 100 | 90 |
| 1.026 | 500 | 95 | 60 | 90 | 0 |
| 1.029 | 500 | 0 | 0 | 95 | 95 |
| 1.032 | 500 | 80 | 60 | 80 | 60 |
| 1.034 | 500 | 95 | 70 | 100 | 40 |
| 1.035 | 500 | 95 | 0 | 95 | 20 |
| 1.037 | 500 | 95 | 60 | 100 | 95 |
| 1.039 | 500 | 95 | 60 | 95 | 0 |
| 1.042 | 500 | 95 | 60 | 100 | 90 |
| 1.043 | 500 | 95 | 0 | 80 | 0 |
| 1.044 | 500 | 95 | 40 | 95 | 90 |
| 1.045 | 500 | 100 | 70 | 100 | 0 |
| 1.048 | 500 | 95 | 80 | 95 | 80 |
| 1.049 | 500 | 95 | 30 | 95 | 10 |
| 1.051 | 500 | 70 | 80 | 100 | 100 |
| 1.052 | 500 | 95 | 80 | 100 | 90 |
| 1.054 | 500 | 95 | 95 | 100 | 30 |
| 1.055 | 500 | 100 | 95 | 0 | 0 |
| 1.056 | 500 | 95 | 90 | 95 | 90 |
| 1.057 | 500 | 95 | 90 | 95 | 80 |
| 1.058 | 500 | 95 | 30 | 100 | 10 |
| 1.061 | 500 | 95 | 80 | 100 | 95 |
| 1.074 | 500 | 0 | 70 | 95 | 80 |
| 1.096 | 500 | 95 | 95 | 95 | 95 |

ECHCG = Echinochloa crus-galli (barnyard grass), ALOMY = Alopecurus myosuroides (slender foxtail), AMARE = Amaranthus retroflexus (redroot pigweed), STEME = Stellaria media (chickweed).

### Example B2

### Herbicidal Action Post Emergence of the Plants (Post-Emergence Action)

Monocotyledonous and dicotyledonous test plants were sown in seed trays in standard compost and were grown for eight days. The trays were watered twice daily or as required. The chemicals were applied by track sprayer to the foliage. The application was carried out with an aqueous suspension of the test substances, prepared as a formulation of 50% acetone in water with 0.5% Tween 20™ (CAS RN 9005-64-5), to achieve a field equivalent of 1000 l/ha. The application rate of the test substances was 500 g/ha. A visual assessment of the herbicidal effect was made at 13 days after application. The following percentage scale was used for assessment: 0, 5, 10, 20, 30, 40, 50, 60, 70, 80, 90, 95, 100 (where O is no damage to plants and 100 is plants are completely dead).

| Compound | Rate | | | | |
|---|---|---|---|---|---|
| No. | (g/ha) | ECHCG | ALOMY | AMARE | STEME |
| 1.001 | 500 | 60 | 80 | 80 | 80 |
| 1.002 | 500 | 95 | 95 | 95 | 90 |
| 1.003 | 500 | 90 | 80 | — | 10 |
| 1.004 | 500 | 90 | 80 | 70 | 60 |
| 1.005 | 500 | 80 | 80 | 80 | 70 |
| 1.006 | 500 | 80 | 70 | 30 | 0 |
| 1.013 | 500 | 50 | 60 | 60 | 80 |
| 1.014 | 500 | 60 | 70 | 70 | 80 |
| 1.017 | 500 | 60 | 70 | 70 | 70 |
| 1.022 | 500 | 80 | 60 | 70 | 80 |
| 1.023 | 500 | 80 | 70 | 80 | 20 |
| 1.025 | 500 | 80 | 70 | 80 | 90 |
| 1.026 | 500 | 70 | 70 | 60 | 0 |
| 1.037 | 500 | 20 | 90 | 95 | 70 |
| 1.051 | 500 | 80 | 70 | 70 | 70 |
| 1.052 | 500 | 70 | 80 | 50 | 10 |
| 1.056 | 500 | 70 | 95 | 70 | 80 |
| 1.057 | 500 | 80 | 90 | 100 | 90 |
| 1.058 | 500 | 70 | 60 | 80 | 0 |
| 1.061 | 500 | 80 | 40 | 40 | 40 |
| 1.096 | 500 | 80 | 90 | 80 | 70 |

ECHCG = Echinochloa crus-galli (barnyard grass), ALOMY = Alopecurus myosuroides (slender foxtail), AMARE = Amaranthus retroflexus (redroot pigweed), STEME = Stellaria media (chickweed).

### Example B3

### Herbicidal Action Prior to Emergence of the Plants (Pre-Emergence Action)

Monocotyledonous and dicotyledonous test plants were sown in sterilised standard soil in seed trays each with 96 cells. The seed trays were stored under controlled conditions in a climatic chamber for one day (cultivation at 23° C. during the day and 17° C. at night; 13 hours of light; 50-60% humidity). The chemicals were applied to the soil surface. The application was carried out with an aqueous suspension of the test substances, prepared as a formulation in water with 10% dimethyl sulfoxide (CAS RN 67-68-5), to achieve a field equivalent of 1000 l/ha. The application rate of the test substances was 1000 g/ha. The plants were grown on in the climatic chamber for 9 days (cultivation at 24° C. during the day and 19° C. at night; 13 hours of light; 50-60% humidity). A visual assessment of the herbicidal effect was made at 9 days after application. The following scale was used for assessment: 0, 1, 2, 3, 4 and 5 (where 0 is no damage to plant and 5 is total damage to plant).

| Compound | Rate | | | | | |
|---|---|---|---|---|---|---|
| No. | (g/ha) | DIGSA | AGSTE | SETIT | POATR | AMARE |
| 1.055 | 1000 | 5 | 4 | 5 | 5 | 2 |
| 1.074 | 1000 | 5 | 5 | 4 | 5 | 3 |
| 1.075 | 1000 | 0 | 5 | 5 | 5 | 1 |
| 1.077 | 1000 | 5 | 5 | 4 | 5 | 3 |
| 1.078 | 1000 | 5 | 5 | 5 | 5 | 4 |
| 1.080 | 1000 | 5 | 5 | 5 | 5 | 4 |
| 1.081 | 1000 | 0 | 5 | 4 | 0 | 5 |
| 1.086 | 1000 | 5 | 5 | 4 | 5 | 5 |
| 1.089 | 1000 | 5 | 5 | 5 | 5 | 5 |
| 1.090 | 1000 | 5 | 5 | 5 | 4 | 3 |
| 1.091 | 1000 | 4 | 5 | 4 | 0 | 4 |
| 1.092 | 1000 | 5 | 5 | 4 | 3 | 3 |
| 1.093 | 1000 | 5 | 5 | 3 | 5 | 4 |
| 1.096 | 1000 | 5 | 5 | 5 | 5 | 5 |
| 1.102 | 1000 | 4 | 5 | 2 | 0 | 2 |
| 1.103 | 1000 | 5 | 5 | 4 | 5 | 4 |
| 1.104 | 1000 | 5 | 5 | 5 | 5 | 5 |
| 1.106 | 1000 | 4 | 0 | 4 | 5 | 2 |
| 1.107 | 1000 | 5 | 0 | 3 | 0 | 3 |
| 1.110 | 1000 | 4 | 0 | 5 | 4 | 4 |
| 1.112 | 1000 | 5 | 5 | 0 | 0 | 4 |
| 1.113 | 1000 | 5 | 5 | 5 | 4 | 5 |
| 1.114 | 1000 | 5 | 5 | 5 | 5 | 5 |
| 1.115 | 1000 | 5 | 5 | 3 | 0 | 3 |
| 1.116 | 1000 | 5 | 5 | 5 | 5 | 5 |
| 1.117 | 1000 | 5 | 5 | 5 | 5 | 5 |
| 1.120 | 1000 | 5 | 5 | 5 | 5 | 5 |
| 1.125 | 1000 | 5 | 5 | 5 | 5 | 5 |
| 1.126 | 1000 | 5 | 4 | 4 | 1 | 4 |
| 1.127 | 1000 | 5 | 5 | 5 | 5 | 5 |
| 1.130 | 1000 | 5 | 5 | 5 | 5 | 5 |
| 1.132 | 1000 | 5 | 5 | 5 | 5 | 5 |
| 1.133 | 1000 | 5 | 5 | 5 | 5 | 5 |

DIGSA = Digitaria sanguinalis (hairy finger-grass); AGSTE = Agrostis tenius (Colonial bentgrass); SETIT = Setaria italica (foxtail millet); POATR = Poa trivialis (rough blue grass); AMARE = Amaranthus retroflexus (redroot pigweed).

What is claimed is:

1. Compounds of formula I 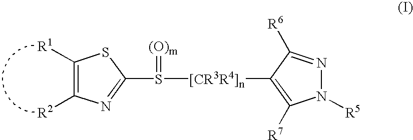
wherein
R¹ and R² are each independently of the other hydrogen, C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCHO, —NHCO—C₁-C₆alkyl, —NHCO—C₁-C₆haloalkyl, —NHCO₂—C₁-C₆alkyl, —NHCO₂—C₁-C₆haloalkyl, —NHCONH—C₁-C₆alkyl, —NHCONH—C₁-C₆haloalkyl, —NHSO₂—C₁-C₆alkyl, —NHSO₂—C₁-C₆haloalkyl, —NHSO₂-phenyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCON—H—C₁-C₆haloalkyl, —OCONH-phenyl or —OCONH-phenyl substituted by one to three R¹¹, or —CONR^{a}R^{b} wherein R^{a} and R^{b} are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R^{a} and R^{b} together form a C₃-C₈alkylene group which optionally contains one oxygen or sulfur atom or one or two amino or C₁-C₆alkylamino groups, or
R¹ and R² together with the carbon atom to which they are bonded form a C₃-C₁₀alkylene group, which optionally contains one or two oxygen or sulfur atoms or one to three amino or C₁-C₆alkylamino groups, and which optionally contains a double bond and optionally is substituted by one to three substituents independently selected from
C₃-C₆cycloalkyl, C₁-C₆haloalkyl, C₁-C₆hydroxyalkyl, pyrrolyl-CH₂—, pyrazolyl-CH₂-triazolyl-CH₂—, imidazolyl-CH₂—, tetrazolyl-CH₂—, indolyl-CH₂—, indazolyl-CH₂—, benzotriazolyl-CH₂—, C₂-C₆alkenyl, C₂-C₆alkynyl, C₂-C₆haloalkenyl, C₂-C₆alkenyloxy, C₂-C₆alkynyloxy, C₁-C₆alkylcarbonyl, C₁-C₆haloalkylcarbonyl, phenylcarbonyl or phenylcarbonyl substituted by one to three R¹¹, phenoxycarbonyl or phenoxycarbonyl substituted by one to three R¹¹, benzyloxycarbonyl or benzyloxycarbonyl substituted by one to three R¹¹, nitro, formyl, carboxyl, halogen, azido, thiocyanato, tri(C₁-C₆alkyl)silyl, C₁-C₆alkylcarbonyl-C₁-C₂alkyl, C₁-C₆alkoxycarbonyl-C₁-C₂alkyl, cyano-C₁-C₂alkyl, C₁-C₆alkylaminocarbonyl-C₁-C₂alkyl, di-C₁-C₆alkylaminocarbonyl-C₁-C₂alkyl, C₁-C₆alkoxy-C₁-C₂alkyl, C₁-C₂alkyl-P(O)(OC₁-C₆alkyl)₂, C₁-C₂alkyl-NO₂, mercapto, phenylthio or phenylthio substituted by one to three R¹¹, pyridylthio, C₁-C₆alkylthio, C₁-C₆haloalkylthio, C₁-C₆alkylthio-C₁-C₆alkyl, C₁-C₆alkylsulfinyl, C₁-C₆haloalkylsulfinyl, C₁-C₆alkylsulfinyl-C₁-C₆alkyl, C₁-C₆alkylsulfonyl, C₁-C₆haloalkylsulfonyl, C₁-C₆alkylsulfonyl-C₁-C₆alkyl, benzylsulfonyl or benzylsulfonyl substituted by one to three R¹¹, phenylsulfinyl or phenylsulfinyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyl or benzyl substituted by one to three R¹¹, benzyloxy or benzyloxy substituted by one to three R¹¹, —CONH—SO₂—C₁-C₆alkyl, —CONH—SO₂—C₁-C₆haloalkyl, —NHCHO, —NHCO—C₁-C₆alkyl, —NHCO—C₁-C₆haloalkyl, —NHCOO—C₁-C₆alkyl, —NHCOO—C₁-C₆haloalkyl, —NHCONH—C₁-C₆alkyl, —NHCONH—C₁-C₆haloalkyl, —NHSO₂—C₁-C₆alkyl, —NHSO₂—C₁-C₆haloalkyl, —NHSO₂-phenyl, —OCO—C₁-C₆alkyl, —OCO—C₁-C₆haloalkyl, —OCO-phenyl or —OCO-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₃-C₆cycloalkyl, C₁-C₆haloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkylamino groups, or phenyl or naphthyl, which is optionally substituted by one to three substituents independently selected from C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkylamino groups, or
a 5- to 10-membered heteroaryl containing one to three nitrogen, oxygen or sulfur atoms, which is optionally benzo-fused, and which is optionally substituted by one to three substituents independently selected from C₁-C₆alkyl, C₃-C₆cycloalkyl, C₁-C₆haloalkyl, C₁-C₆-hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkylamino groups, or
R¹ and R² join together with the carbon atoms to which they are bonded to form a fused aromatic ring, which is optionally substituted by one to three substituents independently selected from C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkylamino groups, or
R¹ and R² join together with the carbon atoms to which they are bonded to form a fused heterocyclic ring containing one to three nitrogen, oxygen or sulfur atoms which is optionally substituted by one to three substituents independently selected from C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R₁₁, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR′R″ wherein R′ and R″ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R′ and R″ together form a C₃-C₈alkylene group which optionally contains one oxygen or sulfur atom or one or two amino or alkylamino groups;
R³ and R⁴ are each independently of the other hydrogen, C₁-C₆alkyl, C₁-C₆haloalkyl, halogen, cyano, C₁-C₆alkoxycarbonyl;
m is 0, 1 or 2;
n is 1, 2 or 3;
R⁵, R⁶ and R⁷ are each independently of the others hydrogen, hydroxyl, mercapto, halogen, C₁-C₁₀alkyl or C₁-C₁₀alkyl substituted by one R⁸, C₁-C₄haloalkyl, C₃-C₈cycloalkyl, C₁-C₁₀alkoxy or C₁-C₁₀alkoxy substituted by one R⁹, C₁-C₄haloalkoxy, C₃-C₈cycloalkyloxy, C₃-C₈cycloalkylC₁-C₃alkoxy, C₁-C₁₀alkylthio or C₁-C₁₀alkylthio substituted by one R⁹, C₁-C₄haloalkylthio, C₂-C₆alkenyl, C₂-C₆haloalkenyl, C₂-C₆alkenyloxy, C₂-C₆alkynyl, C₂-C₆alkynyloxy, C₁-C₁₀alkylsulfinyl or C₁-C₁₀alkylsulfinyl substituted by R⁹, C₁-C₁₀alkylsulfonyl or C₁-C₁₀alkylsulfonyl substituted by one R⁹, C₁-C₄haloalkylsulfinyl, C₁-C₁₀alkylsulfonyloxy substituted by one R⁹, C₁-C₄haloalkylsulfonyl, C₁-C₁₀alkylsulfonyloxy, C₁-C₄haloalkylsulfonyloxy, phenyl or phenyl substituted by one to three R¹⁰, phenoxy or phenoxy substituted by one to three R¹⁰, phenylthio or phenylthio substituted by one to three R¹⁰, heteroaryl or heteroaryl substituted by one to three R¹⁰, heteroaryloxy or heteroaryloxy substituted by one to three R¹⁰, heteroarylthio or heteroarylthio substituted by one to three R¹⁰, phenylsulfinyl or phenylsulfinyl substituted by one to three R¹⁰, phenylsulfonyl or phenylsulfonyl substituted by one to three R¹⁰, heteroarylsulfinyl or heteroarylsulfinyl substituted by one to three R¹⁰, heteroarylsulfonyl or heteroarylsulfonyl substituted by one to three R¹⁰, phenylsulfonyloxy or phenylsulfonyloxy substituted by one to three R¹⁰, C₁-C₆alkylcarbonyl, C₁-C₄haloalkylcarbonyl, C₃-C₈cycloalkylcarbonyl, benzylcarbonyl or benzylcarbonyl substituted by one to three R¹⁰, phenylcarbonyl or phenylcarbonyl substituted by one to three R¹⁰, carboxyl, C₁-C₁₀alkoxycarbonyl, benzyloxycarbonyl or benzyloxycarbonyl substituted by one to three R¹⁰, phenoxycarbonyl or phenoxycarbonyl substituted by one to three R¹⁰, cyano, —CONR^{c}R^{d} (wherein R^{c} and R^{d} are each independently of the other hydrogen, C₁-C₁₀alkyl, phenyl or phenyl substituted by one to three R¹⁰), —O(CO)C₁-C₆alkyl, —O(CO)C₁-C₄haloalkyl, —O(CO)benzyl or —O(CO)benzyl substituted by one to three R¹⁰, —O(CO)phenyl or —O(CO)phenyl substituted by one to three R¹⁰, nitro, or —NR^{c}R^{d} (wherein R^{c} and R^{d} are each independently of the other hydrogen, C₁-C₁₀alkyl, phenyl or phenyl substituted by one to three R¹⁰, C₁-C₆alkylcarbonyl, C₁-C₄haloalkylcarbonyl, benzylcarbonyl or benzylcarbonyl substituted by one to three R¹⁰, phenylcarbonyl or phenylcarbonyl substituted by one to three R¹⁰, C₁-C₁₀alkylsulfonyl, C₁-C₄haloalkylsulfonyl, benzylsulfonyl or benzylsulfonyl substituted by one to three R¹⁰, and phenylsulfonyl or phenylsulfonyl substituted by one to three R¹⁰), or
when R⁵ and R⁷ are substituted both by alkyl, both by alkoxy, alkyl and alkoxy, alkyl and alkylthio, alkyl and alkylsulfonyl, alkyl and monoalkylamino, alkyl and dialkylamino, the two groups optionally form together with the atoms to which they bond, a 5- to 8-membered ring which is optionally substituted by 1 to 4 halogen atoms;
R⁸ is hydroxy, C₃-C₈cycloalkyl or C₃-C₈cycloalkyl substituted by halogen or by C₁-C₁₀alkyl, C₁-C₁₀alkoxy, C₁-C₁₀alkylthio, C₁-C₁₀alkylsulfonyl, C₁-C₁₀alkoxycarbonyl, C₂-C₆haloalkenyl, —NR^{e}R^{f} (wherein R^{e} and R^{f} are each independently of the other hydrogen, C₁-C₁₀alkyl, C₁-C₆alkylcarbonyl, C₁-C₄haloalkylcarbonyl, C₁-C₁₀alkylsulfonyl, C₁-C₄haloalkylsulfonyl), —CONR^{e}R^{f} (wherein R^{e} and R^{f} are each independently of the other hydrogen, C₁-C₁₀alkyl, phenyl or phenyl substituted by one to three R¹⁰), C₁-C₆alkylcarbonyl, C₁-C₄haloalkylcarbonyl, cyano, phenyl or phenyl substituted by one to three R¹⁰, or phenoxy or phenoxy substituted by one to three R¹⁰;
R⁹ is C₁-C₁₀alkoxy, C₁-C₁₀alkoxycarbonyl, phenyl or phenyl substituted by one to three R¹⁰, heteroaryl or heteroaryl substituted by one to three R¹⁰, C₁-C₁₀alkylcarbonyl, C₁-C₁₀haloalkylcarbonyl, cyano, or —CONR^{g}R^{h} (wherein R^{g} and R^{h} are each independently of the other hydrogen, C₁-C₁₀alkyl, phenyl or phenyl substituted by one to three R¹⁰);
R¹⁰ are each independently of the others C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONRⁱR^{k} wherein Rⁱ and R^{k} are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or Rⁱ and R^{k} together form a C₃-C₈alkylene group which optionally contains one oxygen or sulfur atom or one or two amino or C₁-C₆alkylamino groups;
R¹¹ are each independently of the others C₁-C₆haloalkyl, C₁-C₆alkoxycarbonyl, nitro, cyano, formyl, carboxyl or halogen;
and to N-oxides, salts and optical isomers of compounds of formula I, with the proviso that where R¹ and R² are fused to form an unsubstituted benzothiazole ring, R³ and R⁴ are hydrogen, n is 1, R⁵ is 3,5-dichlorobenzylcarbonyl, and R⁶ and R⁷ are methyl, then m cannot be 0.

2. Compounds of formula I according to claim 1 wherein
R¹ and R² are each independently of the other hydrogen, C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCHO, —NHCO—C₁-C₆alkyl, —NHCO—C₁-C₆haloalkyl, —NHCO₂—C₁-C₆alkyl, —NHCO₂—C₁-C₆haloalkyl, —NHCONH—C₁-C₆alkyl, —NHCONH—C₁-C₆haloalkyl, —NHSO₂—C₁-C₆alkyl, —NHSO₂—C₁-C₆haloalkyl, —NHSO₂-phenyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR^{a}R^{b} wherein R^{a} and R^{b} are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R^{a} and R^{b} together form a C₃-C₈alkylene group which optionally contains one oxygen or sulfur atom or one or two amino or C₁-C₆alkylamino groups; and to N-oxides, salts and optical isomers of compounds of formula I.

3. Compounds of formula I according to claim 1 wherein
R¹ and R² are each independently of the other hydrogen, C₁-C₆alkyl, C₃-C₆cycloalkyl, C₁-C₆haloalkyl, C₁-C₆hydroxyalkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl or benzyloxycarbonyl substituted by one to three R¹¹, nitro, cyano, formyl, carboxyl, halogen, azido, thiocyanato, tri(C₁-C₆alkyl)silyl, mercapto, phenylthio or phenylthio substituted by one to three R¹¹, phenylsulfinyl or phenylsulfinyl substituted by one to three R¹¹, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R¹¹, phenylsulfonyl or phenylsulfonyl substituted by one to three R¹¹, hydroxyl, C₁-C₆alkoxy, C₁-C₆haloalkoxy, C₁-C₆alkylsulfonyloxy, C₁-C₆haloalkylsulfonyloxy, phenoxy or phenoxy substituted by one to three R¹¹, benzyloxy or benzyloxy substituted by one to three R¹¹, —CONH—SO₂—C₁-C₆alkyl, —CONH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —O(CO)—C₁-C₆alkyl, —O(CO)—C₁-C₆haloalkyl, —O(CO)-phenyl or —O(CO)-phenyl substituted by one to three R¹¹, —OCONH—C₁-C₆alkyl, —OCONH—C₁-C₆haloalkyl, —OCONH-phenyl or —OCONH-phenyl substituted by one to three R¹¹, or —CONR^{a}R^{b} wherein R^{a} and R^{b} are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R^{a} and R^{b} together form a C₃-C₈alkylene group which optionally contains one oxygen or sulfur atom or one or two amino or C₁-C₆alkylamino groups; and to N-oxides, salts and optical isomers of compounds of formula I.

4. Compounds of formula I wherein
R¹ and R² are each independently of the other hydrogen, C₁-C₆haloalkyl, C₁-C₆alkoxycarbonyl or halogen;
R³ and R⁴ are each independently of the other hydrogen, C₁-C₆alkyl or halogen;
m is 0, 1 or 2;
n is 1;
R⁵, R⁶ and R⁷ are each independently of the others halogen, C₁-C₁₀alkyl, C₁-C₄haloalkyl, C₁-C₁₀alkoxy, C₁-C₁₀alkoxyC₁-C₁₀alkoxy, C₁-C₄haloalkoxy or C₂-C₆alkynyloxy;
and to N-oxides, salts and optical isomers of compounds of formula I.

5. Compounds of formula I wherein
R¹ and R² are each independently of the other hydrogen, C₁-C₆haloalkyl, C₁-C₆alkoxycarbonyl or halogen;
R³ and R⁴ are each independently of the other hydrogen or halogen;
m is 0, 1 or 2;
n is 1;
R⁵, R⁶ and R⁷ are each independently of the others halogen, C₁-C₁₀alkyl, C₁-C₄haloalkyl, C₁-C₁₀alkoxy, C₁-C₁₀alkoxyC₁-C₁₀alkoxy, C₁-C₄haloalkoxy or C₂-C₆alkynyloxy;
and to N-oxides, salts and optical isomers of compounds of formula I.

6. Compounds of formula I wherein
R¹ and R² are each independently of the other hydrogen, C₁-C₆haloalkyl, C₁-C₆alkoxycarbonyl or halogen;
R³ and R⁴ are both hydrogen;
m is 0, 1 or 2;
n is 1;
R⁵, R⁶ and R⁷ are each independently of the others halogen, C₁-C₁₀alkyl, C₁-C₄haloalkyl or C₁-C₄haloalkoxy;
and to N-oxides, salts and optical isomers of compounds of formula I.

7. A compound of formula II 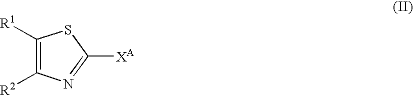
wherein R¹ is chloro, R² is hydrogen and X^{A} is methylsulfonate.

8. A process for the preparation of compounds of formula Ih in which R¹, R², R³, R⁴, R⁵, R⁶ and R⁷ are defined as in claim 1, and m is 1 or 2, 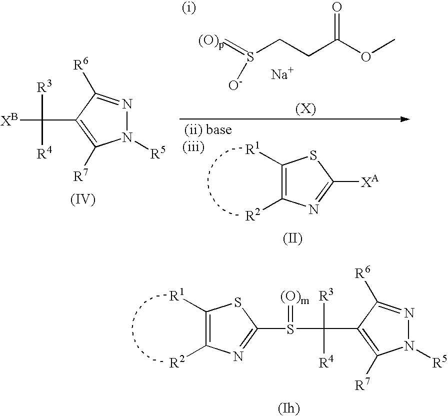
wherein a compound of formula IV in which X^{B} is a leaving group is sequentially reacted with a compound X in which p is 0 or 1 in the presence of a diluent and a base and with a compound II in which X^{A} is a leaving group.

9. A process for the preparation of compounds of formula Ih in which R¹, R², R³, R⁴, R⁵, R⁶ and R⁷ are defined as in claim 1, and m is 1 or 2, 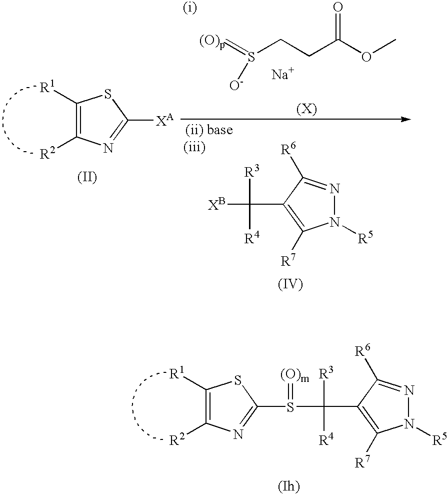
wherein a compound of formula II in which X^{A} is a leaving group is sequentially reacted with a compound X in which p is 0 or 1 in the presence of a diluent and a base and with a compound IV in which X^{B} is a leaving group.

10. A process for the preparation of compounds of formula Ih in which R¹, R², R³, R⁴, R⁵, R⁶ and R⁷ are defined as in claim 1, and m is 1 or 2, 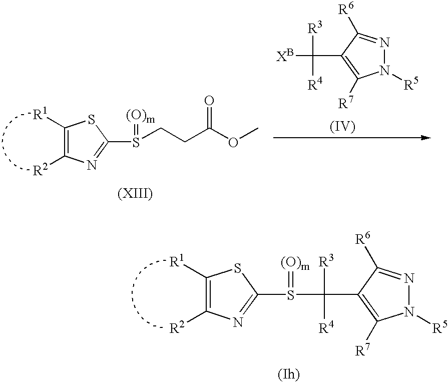
wherein a compound of formula XIII is reacted with a compound IV in which X^{B} is a leaving group in the presence of a diluent and in the presence of a base.

11. A process for the preparation of compounds of formula XIII in which R¹ and R² are defined as in claim 1, and m is 1 or 2, 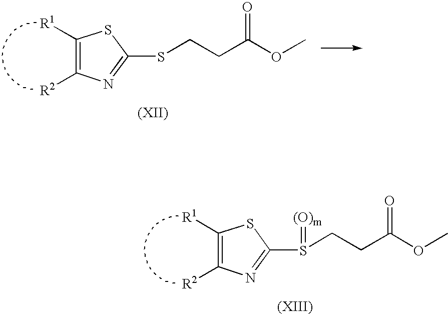
wherein a compound of formula XII is oxidised with an oxidising agent optionally in the presence of a diluent.

12. A process for the preparation of compounds of formula XII in which R¹ and R² are defined as in claim 1, 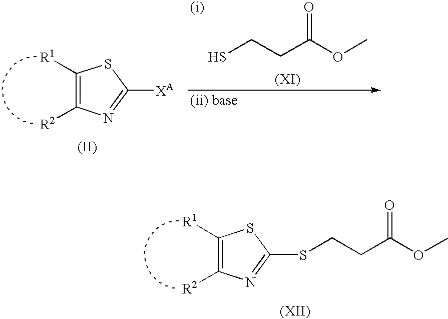
wherein a compound of formula II in which X^{A} is a leaving group is sequentially reacted with a compound XI in the presence of a diluent and a base.

13. A process for the preparation of compounds of formula IVa wherein a compound of formula XIV is reacted with reagent XV in the presence of a diluent 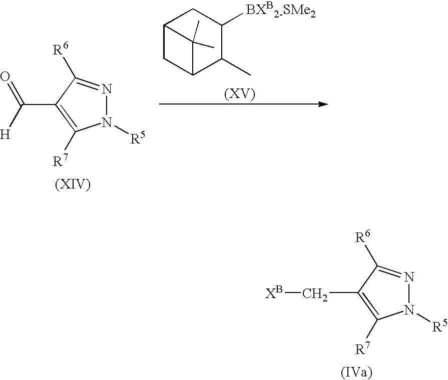
wherein R⁵, R⁶ and R⁷ are defined as in claim 1, and X^{B} is a halogen atom.

14. A herbicidal composition which comprises a herbicidally effective amount of a compound of formula I in addition to formulation adjuvants.

15. A method of controlling grasses and weeds in crops of useful plants, which comprises applying a herbicidally effective amount of a compound of formula I, or of a composition comprising such a compound, to the plants or to the locus thereof.

16. A composition according to claim 14, which comprises a further herbicide in addition to the compound of formula I.

17. A composition according to claim 14, which comprises a safener in addition to the compound of formula I.
